SEQUENCE LISTING (146 sequences)

SEQ ID NO: 1 - protein, 16 aa, Plasmodium berghei
DPPPNPNDDPPPNPND

SEQ ID NO: 2 - protein, 12 aa, Plasmodium berghei
NDDSYIPSAEKI

SEQ ID NO: 3 - protein, 16 aa, Artificial Sequence
  A synthetic peptide vaccine to P. berghei containing a tandemly repeating domain has been produced using a “multiple antigenic peptide system” in which the synthetic peptides are linked to a lattice matrix of lysines
DPPPPNPNDPPPPNPN

SEQ ID NO: 4 - protein, 12 aa, Artificial Sequence
  constructs (TB4 and BT4) containing four copies of both the B cell epitope and a T cell epitope
KQIRDSITEEWS

SEQ ID NO: 5 - protein, 9 aa, Artificial Sequence
  recombinant Hepatitis B core antigen (HBcAg) VLP incorporating the immunodominant B cell epitopes for P. yoelii
SYVPSAEQI

SEQ ID NO: 6 - protein, 9 aa, Artificial Sequence
  cloned P. berghei cytotoxic CD4+ T cell epitope in a Ty vector
SYIPSAEKI

SEQ ID NO: 7 - protein, 17 aa, Plasmodium falciparum
DPPPPNPNDPPPPNPND

SEQ ID NO: 8 - protein, 20 aa, Plasmodium falciparum
EYLNKIQNSLSTEWSPCSVT

SEQ ID NO: 9 - protein, 8 aa, Plasmodium falciparum
DPNANPNV

SEQ ID NO: 10 - skipped

SEQ ID NO: 11 - protein, 17 aa, Artificial Sequence
  ELISA unit = dilution to give OD > geometric mean + 3 SD of their respective pre-immune sera (ELISA using the synthetic peptide (DPPPNDVP)2D (SEQ ID NO 11) as the plague antigen.
DPPPNDVPDPPPNDVPD

SEQ ID NO: 12 - protein, 14 aa, Plasmodium berghei
KIYNRNTVNRLLAD

SEQ ID NO: 13 - protein, 8 aa, Plasmodium berghei
DPPPPNPN

SEQ ID NO: 14 - protein, 8 aa, Plasmodium berghei
SYPSAEKI

SEQ ID NO: 15 - protein, 44 aa, Artificial Sequence
  replacement of all the a and d positions of the heptad repeat of a coiled-coil predicted from P. falciparum with tryptophan residues, can force it into a highly stable pentameric oligomerization state substituting a phenylalanine and tyrosine
NNFDNYNNNCDNYYNNFDNYNNNFDNYNNNFDNYNNNFDNYNNN

SEQ ID NO: 16 - protein, 46 aa, Artificial Sequence
  replacement of all the a and d positions of the heptad repeat of a coiled-coil predicted from P. falciparum with tryptophan residues, can force it into a highly stable pentameric oligomerization state substituting a phenylalanine and tyrosine
HNHYDNRYNHHDNRYNHHDNRYNHHDNRYNHHDNRYNHHDNRYNNK

SEQ ID NO: 17 - skipped

SEQ ID NO: 18 - protein, 66 aa, Artificial Sequence
  nanoparticle core without a fused epitope (N-Empty) - NCP1. COMP with trimeric linker peptide sequence.
GSDEMLRELQETNAALQDVRELLRQQVRQITFLKCLLMGGRLLCRLEELERRLEELERRLEELERA

SEQ ID NO: 19 - protein, 107 aa, Artificial Sequence
  SAPN displaying the P. berghei CSP B cell epitope peptide (N-PbCSP)
GSDEMLRELQETNAALQDVRELLRQQVRQITFLKCLLMGGRLLCRLEELERRLEELERRLEELERAINTVDLELAALRRRLEELARGGSGDPPPPNPNDPPPPNPND

SEQ ID NO: 20 - protein, 87 aa, Artificial Sequence
  SAPN displaying the P. facliparum apial membrane anigen (N-PfAMA)
GSDEMLRELQETNAALQDVRELLRQQVRQITFLKCLLMGGRLLCRLEELERRLEELERRLEELERARGGIPSTAFTDIAWVRLPNHY

SEQ ID NO: 21-22 - skipped (2 entries)

SEQ ID NO: 23 - protein, 45 aa, Artificial Sequence
  .  Core SAPN composed of a pentameric coiled-coil, a linker sequence and a trimerization domain and a B-Cell epitope of malaria with an insertion of a first extension of the trimeric coiled-coil.
LERAINTVDLELAALRRRLEELARGGSGDPPPPNPNDPPPPNPND

SEQ ID NO: 24 - protein, 66 aa, Artificial Sequence
  Core SAPN composed of a pentameric coiled-coil, a linker sequence and a trimerization domain and a B-Cell epitope of malaria with an insertion of a first and second extension of the trimeric coiled-coil.
LERAISAIKADLSALKANLASLQADINTVDLELAALRRRLEELARGGSGDPPPPNPNDPPPPNPND

SEQ ID NO: 25 - protein, 107 aa, Artificial Sequence
  Plasmodium trimeric coiled-coil original sequence linked with pentamerization domains: 1) the original pentamerization sequence
GSDEMLRELQETNAALQDVRELLRQQVRQITFLRALLMGGRLLARLEELERRLEELERRLEELERAINTVDLELAALRRRLEELARGGSGDPPPPNPNDPPPPNPND

SEQ ID NO: 26 - protein, 108 aa, Artificial Sequence
  Plasmodium trimeric coiled-coil original sequence linked with pentamerization domains: 2) the published tryptophan zipper [62], and the P. falciparum coiled-coil sequences,
GSWQTWNAKWDQWSNDWNAWRSDWQAWKDDWARLRALLMGGRLLARLEELERRLEELERRLEELERAINTVDLELAALRRRLEELARGGSGDPPPPNPNDPPPPNPND

SEQ ID NO: 27 - protein, 108 aa, Artificial Sequence
  Plasmodium trimeric coiled-coil original sequence linked with pentamerization domains: 3) PFI1180w
GSWDNWNNNWDNWYNNWDNWNNNWDNWNNNWDNLRALLMGGRLLARLEELERRLEELERRLEELERAINTVDLELAALRRRLEELARGGSGDPPPPNPNDPPPPNPND

SEQ ID NO: 28 - protein, 108 aa, Artificial Sequence
  Plasmodium trimeric coiled-coil original sequence linked with pentamerization domains: 4) PFF0535c.
GSWNHWDNRWNHWDNRWNHWDNRWNHWDNRWNHLRALLMGGRLLARLEELERRLEELERRLEELERAINTVDLELAALRRRLEELARGGSGDPPPPNPNDPPPPNPND

SEQ ID NO: 29 - protein, 107 aa, Artificial Sequence
  Plasmodium trimeric coiled-coil PF11_0207 linked with pentamerization domains: 1) the original pentamerization sequence,
GSDEMLRELQETNAALQDVRELLRQQVRQITFLRALLMGGRLLARLKEVKEEIKEVKEEIKEVKEEIKEVKEEIKEEIKEVKELARGGSGDPPPPNPNDPPPPNPND

SEQ ID NO: 30 - protein, 108 aa, Artificial Sequence
  Plasmodium trimeric coiled-coil PF11_0207 linked with pentamerization domains: 2) the published tryptophan zipper and the P. falciparum coiled-coil sequences
GSWQTWNAKWDQWSNDWNAWRSDWQAWKDDWARLRALLMGGRLLARLKEVKEEIKEVKEEIKEVKEEIKEVKEEIKEEIKEVKELARGGSGDPPPPNPNDPPPPNPND

SEQ ID NO: 31 - protein, 108 aa, Artificial Sequence
  Plasmodium trimeric coiled-coil PF11_0207 linked with pentamerization domains: 3) PFI1180w
GSWDNWNNNWDNWYNNWDNWNNNWDNWNNNWDNLRALLMGGRLLARLKEVKEEIKEVKEEIKEVKEEIKEVKEEIKEEIKEVKELARGGSGDPPPPNPNDPPPPNPND

SEQ ID NO: 32 - protein, 108 aa, Artificial Sequence
  Plasmodium trimeric coiled-coil  linked with PF11_207 pentamerization domains: 4) PFF0535c.
GSWNHWDNRWNHWDNRWNHWDNRWNHWDNRWNHLRALLMGGRLLARLKEVKEEIKEVKEEIKEVKEEIKEVKEEIKEEIKEVKELARGGSGDPPPPNPNDPPPPNPND

SEQ ID NO: 33 - protein, 93 aa, Artificial Sequence
  Plasmodium trimeric coiled-coil PF14_240 linked with pentamerization domains: 1) the original pentamerization sequence
GSDEMLRELQETNAALQDVRELLRQQVRQITFLRALLMGGRLLARLENLNNEIHEIEKMWLFIKKKEEILARGGSGDPPPPNPNDPPPPNPND

SEQ ID NO: 34 - protein, 94 aa, Artificial Sequence
  Plasmodium trimeric coiled-coil PF14_0240 linked with pentamerization domains: 2) the published tryptophan zipper [62], and the P. falciparum coiled-coil sequences
GSWQTWNAKWDQWSNDWNAWRSDWQAWKDDWARLRALLMGGRLLARLENLNNEIHEIEKMWLFIKKKEEILARGGSGDPPPPNPNDPPPPNPND

SEQ ID NO: 35 - protein, 94 aa, Artificial Sequence
  Plasmodium trimeric coiled-coil PF14_0240 linked with pentamerization domains: 3) PFI1180w
GSWDNWNNNWDNWYNNWDNWNNNWDNWNNNWDNLRALLMGGRLLARLENLNNEIHEIEKMWLFIKKKEEILARGGSGDPPPPNPNDPPPPNPND

SEQ ID NO: 36 - protein, 94 aa, Artificial Sequence
  Plasmodium trimeric PF14_0240 coiled-coil linked with pentamerization domains: 4) PFF0535c.
GSWNHWDNRWNHWDNRWNHWDNRWNHWDNRWNHLRALLMGGRLLARLENLNNEIHEIEKMWLFIKKKEEILARGGSGDPPPPNPNDPPPPNPND

SEQ ID NO: 37 - protein, 121 aa, Artificial Sequence
  Plasmodium trimeric coiled-coil PF14_0535 linked with pentamerization domain: 1) the original pentamerization sequence
GSDEMLRELQETNAALQDVRELLRQQVRQITFLRALLMGGRLLARLNNIDDHINNIDDYINNIDDHINNIDDHINNIDDHINNIDDHINNIDDHINNVARGGSGDPPPPNPNDPPPPNPND

SEQ ID NO: 38 - protein, 122 aa, Artificial Sequence
  Plasmodium trimeric coiled-coil  PF14_0535 linked with pentamerization domain: 2) the published tryptophan zipper [62], and the P. falciparum coiled-coil sequences,
GSWQTWNAKWDQWSNDWNAWRSDWQAWKDDWARLRALLMGGRLLARLNNIDDHINNIDDYINNIDDHINNIDDHINNIDDHINNIDDHINNIDDHINNVARGGSGDPPPPNPNDPPPPNPND

SEQ ID NO: 39 - protein, 122 aa, Artificial Sequence; identical to SEQ ID NO: 38
  Plasmodium trimeric coiled-coil PF14_0535 linked with pentamerization domain: 3) PFI1180w

SEQ ID NO: 40 - protein, 122 aa, Artificial Sequence
  Plasmodium trimeric coiled-coil PF14_0535 linked with pentamerization domain: 4) PFF0535c
GSWNHWDNRWNHWDNRWNHWDNRWNHWDNRWNHLRALLMGGRLLARLNNIDDHINNIDDYINNIDDHINNIDDHINNIDDHINNIDDHINNIDDHINNVARGGSGDPPPPNPNDPPPPNPND

SEQ ID NO: 41 - protein, 107 aa, Artificial Sequence
  Plasmodium trimeric coiled-coil chimeric of PF11_0207, PF11_0240 and PF14_0535 linked with pentamerization domain: 1) the original pentamerization sequence
GSDEMLRELQETNAALQDVRELLRQQVRQITFLRALLMGGRLLARLNNIDDHINNLNNEIHEIEKMWLFVKEEIKEVKEEIKELARGGSGDPPPPNPNDPPPPNPND

SEQ ID NO: 42 - protein, 108 aa, Artificial Sequence
  Plasmodium trimeric coiled-coil  chimeric of PF11_0207, PF11_0240 and PF14_0535 linked with pentamerization domain: 2) the published tryptophan zipper [62], and the P. falciparum coiled-coil sequences,
GSWQTWNAKWDQWSNDWNAWRSDWQAWKDDWARLRALLMGGRLLARLNNIDDHINNLNNEIHEIEKMWLFVKEEIKEVKEEIKELARGGSGDPPPPNPNDPPPPNPND

SEQ ID NO: 43 - protein, 108 aa, Artificial Sequence
  Plasmodium trimeric coiled-coil chimeric of PF11_0207, PF11_0240 and PF14_0535 llinked with pentamerization domain: 3) PFI1180w
GSWDNWNNNWDNWYNNWDNWNNNWDNWNNNWDNLRALLMGGRLLARLNNIDDHINNLNNEIHEIEKMWLFVKEEIKEVKEEIKELARGGSGDPPPPNPNDPPPPNPND

SEQ ID NO: 44 - protein, 108 aa, Artificial Sequence
  Plasmodium trimeric coiled-coil chimeric of PF11_0207, PF11_0240 and PF14_0535 l  linked with pentamerization domain: 4) PFF0535c
GSWNHWDNRWNHWDNRWNHWDNRWNHWDNRWNHLRALLMGGRLLARLNNIDDHINNLNNEIHEIEKMWLFVKEEIKEVKEEIKELARGGSGDPPPPNPNDPPPPNPND

SEQ ID NO: 45 - protein, 107 aa, Artificial Sequence
  Plasmodium trimeric coiled-coil original sequence linked with pentamerization domains: 1) the original pentamerization sequence
GSDEMLRELQETNAALQDVRELLRQQVRQITFLRCLLMGGRLLCRLEELERRLEELERRLEELERAINTVDLELAALRRRLEELARGGSGDPPPPNPNDPPPPNPND

SEQ ID NO: 46 - protein, 107 aa, Artificial Sequence
  Plasmodium trimeric coiled-coil new sequence linked with pentamerization domains: 1) the original pentamerization sequence
GSDEMLRELQETNAALQDVRELLRQQVRQITFLRCLLMGGRIKEEIKEVKEEIKEVKEEIKEVKEEIKEVKEEIKEEIKEVKELARGGSGDPPPPNPNDPPPPNPND

SEQ ID NO: 47 - protein, 107 aa, Artificial Sequence
  Plasmodium trimeric coiled-coil new sequence linked with pentamerization domain
GSDNWNNNWDNWYNNWDNWNNNWDNWNNNWDNWNNNWMGGRIKEEIKEVKEEIKEVKEEIKEVKEEIKEVKEEIKEEIKEVKELARGGSGDPPPPNPNDPPPPNPND

SEQ ID NO: 48 - protein, 55 aa, Plasmodium falciparum
IKDEVTKIKQISDNIIKIDNVEELNKDIENLNNELHEIEKMWLFIKKKEEILNEF

SEQ ID NO: 49 - protein, 62 aa, Plasmodium falciparum
MTEAENIKIEKPDFDAYNEKLGSISQSIDEIKKKIDNLQKEIKVANKGKEEYNKKKKDIVTK

SEQ ID NO: 50 - protein, 110 aa, Plasmodium falciparum
LNHKLKIDNIYEIIEHNLRDIDKINENINRIKNNIKKLNDDINELLDKKNEINFLDNIQKILQKLEEDKINIYKNLNNLNQELNQLRDNINTNFEKYEHIENKIENCQKK

SEQ ID NO: 51 - protein, 168 aa, Plasmodium falciparum
  MISC_FEATURE (18)..(18): where X is any naturally occurring amino acid
  MISC_FEATURE (47)..(47): where X is any naturally occurring amino acid
  MISC_FEATURE (76)..(76): where X is any naturally occurring amino acid
  MISC_FEATURE (142)..(142): where X is any naturally occurring amino acid
DIQERKEEIKEEFKEEVXEEIKEIKEEIKEVKEEIKEEIKEEIKEVXEEIKEEIKEEIKEVKEEIKEVKEEIKEVXEEIKEVKEEIKEEIKEEIKEEIKEVREEIKEEVKEEIEVKEEIKEVKEEIKEEVKEEIKEVKEEIXEVKEEIKEEIKEVKEEIKEEVKEEIK

SEQ ID NO: 52 - protein, 55 aa, Plasmodium falciparum
DDDQRSMIGFNTYVKDKFDQLRBLKKQVDHLQKQITNYTKINLDDFIVLAPGLCE

SEQ ID NO: 53 - protein, 55 aa, Plasmodium falciparum
EQFDESFEANKKKNNEMDETINKLKKDINKLNEKIEKYDNFMKMKRKEIDIDFSD

SEQ ID NO: 54 - protein, 55 aa, Plasmodium falciparum
FSNEKMNDYNIMENNNYKSKIDELENKIEKLKNELSKNSHNNNNINYNNDYNNSY

SEQ ID NO: 55 - protein, 55 aa, Plasmodium falciparum
KKHEKDLLSKDKEIEEKNKKIKELNNDIKKLQDEILVYKKQSNAQQVDHKKKSWI

SEQ ID NO: 56 - protein, 55 aa, Plasmodium falciparum
KKRNVNGYEEYMSQDISTNNINNINNNIHNIHNNIHDNSNNNTCYDNYSKYTRNR

SEQ ID NO: 57 - protein, 55 aa, Plasmodium falciparum
FLYKQLKYCIEDEIQCYYSEVKKLNDDIHNIKRNVMNIIFKSINDVCTMPDPYQI

SEQ ID NO: 58 - protein, 76 aa, Plasmodium falciparum
NNIDDHINNIDDHINNIDDYINNIDDHINNIDDHINNIDDHINNIDDHINNIDDHINNVDIINKKDNVLNVDYIKE

SEQ ID NO: 59 - protein, 55 aa, Plasmodium falciparum
IKKLDNKDEREPRISNILFMLNNIKKEIENVKENILKYIDKNNLDVNTYTNNITL

SEQ ID NO: 60 - protein, 55 aa, Plasmodium falciparum
ENYKKQKEKETNDLSSTDEIINEIEKKIEDIEKNINITKENLKEKENKITEKQSS

SEQ ID NO: 61 - protein, 55 aa, Plasmodium falciparum
IHVNNKTEQNKDNGHIENNNINNINNNINNINNNINNINNNINNINNNVNNYYYI

SEQ ID NO: 62 - protein, 55 aa, Plasmodium falciparum
KDINTFLEDNECKIVIAKKKIDNLKKQLEDIDDQLQNSKTPELTKEEENELNTLK

SEQ ID NO: 63 - protein, 55 aa, Plasmodium falciparum
SQGVSTANNNDNNVNNMDNNVNNVDNNVNNVDNNLNNVDNNVNNNNVKSSGVEKG

SEQ ID NO: 64 - protein, 55 aa, Plasmodium falciparum
YTVYNKLKELKEEEKKEKSHIEELNNNIEKIDDELRNIYDTKSSTVAKKNGCAST

SEQ ID NO: 65 - protein, 55 aa, Plasmodium falciparum
NNYNNFYSKKEQPQNKQENNLNNIKEQLNNIHENLLLQYGHEINGDDFFMDPLDD

SEQ ID NO: 66 - protein, 55 aa, Plasmodium falciparum
NFVNNYINENILNLKSVDNYLEKINNKIKDLDDNINDRIQKYILMKNEYEKMFKR

SEQ ID NO: 67 - protein, 61 aa, Plasmodium falciparum
LNHKLKIDNITEIIEHNLRDIDKINENINRIKNNIKKLNDDINELLDKKNEINFLEKNEES

SEQ ID NO: 68 - protein, 61 aa, Plasmodium falciparum
EIKEEIKEVKEEIKEEIKEEIKEVKEEIKEVKEEIKEVKEEIKEVKEEIKEEIKEVKEEIK

SEQ ID NO: 69 - protein, 61 aa, Plasmodium falciparum
NNIDDHINNIDDHINNIDDYINNIDDHINNIDDHINNIDDHINNIDDHINNIDDHINNVDI

SEQ ID NO: 70 - protein, 61 aa, Plasmodium falciparum
IHVNNKTEQNKDNGHIENNNINNINNNINNINNNINNINNNINNINNNVNNYYYIDNGDGN

SEQ ID NO: 71 - protein, 61 aa, Plasmodium falciparum
SQGVSTANNNDNNVNNMDNNVNNVDNNVNNVDNNLNNVDNNVNNNNVKSSGVEKGFMDGSS

SEQ ID NO: 72 - protein, 55 aa, Plasmodium berghei
HKENNTLPDLNNVDKKTKKLIDQLRKELEEIKKELNNKRSSEVEIQPIKDKIITK

SEQ ID NO: 73 - protein, 55 aa, Plasmodium berghei
HKESNTSLDFKNVDSKTKKIINELRKELEEVKKDLDNKRNDELAIQPILDKKIAK

SEQ ID NO: 74 - protein, 55 aa, Plasmodium berghei
HKGSSTSLDLKNVDSKTKKIINELRKELEEVKKQIADKTNGELAIQPILDKKIIK

SEQ ID NO: 75 - protein, 55 aa, Plasmodium berghei
HKGSSTSLDLKNVDSKTKKIINELRKELEEVKKELDNKRNDELAIQPIENKKIIK

SEQ ID NO: 76 - protein, 55 aa, Plasmodium berghei
HKENNTLLDLNTVDKKTKKLINELRKELEEVKKEIDNKMKGEFAIQSIDDKIIIK

SEQ ID NO: 77 - protein, 55 aa, Plasmodium berghei
NISSTPEEDIKKKNNIVKKKIKNIKKRIKEINEELHDISNFFLEDKRKREKLTHE

SEQ ID NO: 78 - protein, 55 aa, Plasmodium berghei
HKESNTSLDLKNVDSKTKTLIKELRKELEELKKHISDKTNGELSIQLIDDKIIIK

SEQ ID NO: 79 - protein, 41 aa, Plasmodium berghei
MSCIDVKQENVQNIDKNVNKIKDDIKTDTNKKEGNNENGTR

SEQ ID NO: 80 - protein, 55 aa, Plasmodium berghei
HKESNTSLDLKNVDSKANKIINELRKELEEVKKDISDKMNGELAIQPIHDKIIMK

SEQ ID NO: 81 - protein, 55 aa, Plasmodium berghei
HKGSKTSLYFKNVDSKTKKIIKELRKELEEVKKELDNKRNDELAIQPIENKKIIK

SEQ ID NO: 82 - protein, 55 aa, Plasmodium berghei
HIESNTSLDFKNVDSKTKKIINELRKELEELKKQISDKTNGELAIQPIDDKIIIK

SEQ ID NO: 83 - protein, 55 aa, Plasmodium berghei
KHMVSMQQEKENELAKKEKDLQHLDNKIEEINKNLINETVAYETIKKELVTNKTT

SEQ ID NO: 84 - protein, 55 aa, Plasmodium berghei
HKGSSTSLDLKNVDSKTKKKINELRKELEEVKKELDNKRNDELAIQPIENKKIIK

SEQ ID NO: 85 - protein, 55 aa, Plasmodium berghei
HKGSKTSLDLKNVDSKTKKKINELRKELEEVKKELDNKRNDELAIQPIENKKIIK

SEQ ID NO: 86 - protein, 55 aa, Plasmodium berghei
FDDCFLKNNYLINHNFINYNVKNINKNIQKVDRKLKKEKYAYDALLHNSLILENS

SEQ ID NO: 87 - protein, 55 aa, Plasmodium berghei
HKESNTSLDLKNVDSKANTIINELRKELEEVRKDISDKMNGVLAKQRIHDEIMIH

SEQ ID NO: 88 - protein, 55 aa, Plasmodium berghei; identical to SEQ ID NO: 75

SEQ ID NO: 89 - protein, 55 aa, Plasmodium berghei; identical to SEQ ID NO: 84

SEQ ID NO: 90 - protein, 55 aa, Plasmodium berghei
QKSKNEYTMLEAEVLGTEIKIKHIKNNIKKIEKNLERQQEIIYKFDFHTQILTKK

SEQ ID NO: 91 - protein, 55 aa, Plasmodium berghei
HKGSNTSLDLKNVDSKTKKIINELRKKLEELKKQISDKTNGELAIQPIDDKIIIK

SEQ ID NO: 92 - protein, 8 aa, Plasmodium falciparum
NANPNANP

SEQ ID NO: 93 - protein, 12 aa, Plasmodium falciparum
NANPNANPNANP

SEQ ID NO: 94 - protein, 16 aa, Plasmodium falciparum
NANPNANPNANPNANP

SEQ ID NO: 95 - protein, 102 aa, Artificial Sequence
  Plasmodium falciparum NANP3 epitpoe linked with pentamerization domains
GSDEMLRELQETNAALQDVRELLRQQVRQITFLRALLMGGRLLARLEELERRLEELERRLEELERAINTVDLELAALRRRLEELARGGSGNANPNANPNANP

SEQ ID NO: 96 - protein, 103 aa, Artificial Sequence
  Plasmodium falciparum NANP3 epitpoe linked with pentamerization domains
GSWQTWNAKWDQWSNDWNAWRSDWQAWKDDWARLRALLMGGRLLARLEELERRLEELERRLEELERAINTVDLELAALRRRLEELARGGSGNANPNANPNANP

SEQ ID NO: 97 - protein, 106 aa, Artificial Sequence
  Plasmodium falciparum NANP3 epitpoe linked with pentamerization domains
GSWDNWNNNWDNWYNNWDNWNNNWDNWNNNWDNLRALLMGGRLLARLEELERRLEELERRLEELERAINTVDLELAALRRRLEELARGGSGNPNNANPNANPNANP

SEQ ID NO: 98 - protein, 103 aa, Artificial Sequence
  Plasmodium falciparum NANP3 epitpoe linked with pentamerization domains
GSWNHWDNRWNHWDNRWNHWDNRWNHWDNRWNHLRALLMGGRLLARLEELERRLEELERRLEELERAINTVDLELAALRRRLEELARGGSGNANPNANPNANP

SEQ ID NO: 99 - protein, 102 aa, Artificial Sequence
  Plasmodium falciparum NANP3 epitpoe linked with pentamerization domains
GSDEMLRELQETNAALQDVRELLRQQVRQITFLRALLMGGRLLARLKEVKEEIKEVKEEIKEVKEEIKEVKEEIKEEIKEVKELARGGSGNANPNANPNANP

SEQ ID NO: 100 - protein, 103 aa, Artificial Sequence
  Plasmodium falciparum NANP3 epitpoe linked with pentamerization domains
GSWQTWNAKWDQWSNDWNAWRSDWQAWKDDWARLRALLMGGRLLARLKEVKEEIKEVKEEIKEVKEEIKEVKEEIKEEIKEVKELARGGSGNANPNANPNANP

SEQ ID NO: 101 - protein, 103 aa, Artificial Sequence
  Plasmodium falciparum NANP3 epitpoe linked with pentamerization domains
GSWDNWNNNWDNWYNNWDNWNNNWDNWNNNWDNLRALLMGGRLLARLKEVKEEIKEVKEEIKEVKEEIKEVKEEIKEEIKEVKELARGGSGNANPNANPNANP

SEQ ID NO: 102 - protein, 103 aa, Artificial Sequence
  Plasmodium falciparum NANP3 epitpoe linked with pentamerization domains
GSWNHWDNRWNHWDNRWNHWDNRWNHWDNRWNHLRALLMGGRLLARLKEVKEEIKEVKEEIKEVKEEIKEVKEEIKEEIKEVKELARGGSGNANPNANPNANP

SEQ ID NO: 103 - protein, 88 aa, Artificial Sequence
  Plasmodium falciparum NANP3 epitpoe linked with pentamerization domains
GSDEMLRELQETNAALQDVRELLRQQVRQITFLRALLMGGRLLARLENLNNEIHEIEKMWLFIKKKEEILARGGSGNANPNANPNANP

SEQ ID NO: 104 - protein, 89 aa, Artificial Sequence
  Plasmodium falciparum NANP3 epitpoe linked with pentamerization domains
GSWQTWNAKWDQWSNDWNAWRSDWQAWKDDWARLRALLMGGRLLARLENLNNEIHEIEKMWLFIKKKEEILARGGSGNANPNANPNANP

SEQ ID NO: 105 - protein, 89 aa, Artificial Sequence
  Plasmodium falciparum NANP3 epitpoe linked with pentamerization domains
GSWDNWNNNWDNWYNNWDNWNNNWDNWNNNWDNLRALLMGGRLLARLENLNNEIHEIEKMWLFIKKKEEILARGGSGNANPNANPNANP

SEQ ID NO: 106 - protein, 89 aa, Artificial Sequence
  Plasmodium falciparum NANP3 epitpoe linked with pentamerization domains
GSWNHWDNRWNHWDNRWNHWDNRWNHWDNRWNHLRALLMGGRLLARLENLNNEIHEIEKMWLFIKKKEEILARGGSGNANPNANPNANP

SEQ ID NO: 107 - protein, 115 aa, Artificial Sequence
  Plasmodium falciparum NANP3 epitpoe linked with pentamerization domains
GSDEMLRELQETNAALQDVRELLRQQVRQITFLRALLMGGRLLARLNNIDDHINNIDDYINNIDDHINNIDDHINNIDDHINNIDDHINNIDDHINNVARGGSGNANPNANPNAN

SEQ ID NO: 108 - protein, 117 aa, Artificial Sequence
  Plasmodium falciparum NANP3 epitpoe linked with pentamerization domains
GSWQTWNAKWDQWSNDWNAWRSDWQAWKDDWARLRALLMGGRLLARLNNIDDHINNIDDYINNIDDHINNIDDHINNIDDHINNIDDHINNIDDHINNVARGGSGNANPNANPNANP

SEQ ID NO: 109 - protein, 117 aa, Artificial Sequence
  Plasmodium falciparum NANP3 epitpoe linked with pentamerization domains
GSWDNWNNNWDNWYNNWDNWNNNWDNWNNNWDNLRALLMGGRLLARLNNIDDHINNIDDYINNIDDHINNIDDHINNIDDHINNIDDHINNIDDHINNVARGGSGNANPNANPNANP

SEQ ID NO: 110 - protein, 117 aa, Artificial Sequence
  Plasmodium falciparum NANP3 epitpoe linked with pentamerization domains
GSWNHWDNRWNHWDNRWNHWDNRWNHWDNRWNHLRALLMGGRLLARLNNIDDHINNIDDYINNIDDHINNIDDHINNIDDHINNIDDHINNIDDHINNVARGGSGNANPNANPNANP

SEQ ID NO: 111 - protein, 102 aa, Artificial Sequence
  Plasmodium falciparum NANP3 epitpoe linked with pentamerization domains
GSDEMLRELQETNAALQDVRELLRQQVRQITFLRALLMGGRLLARLNNIDDHINNLNNEIHEIEKMWLFVKEEIKEVKEEIKELARGGSGNANPNANPNANP

SEQ ID NO: 112 - protein, 103 aa, Artificial Sequence
  Plasmodium falciparum NANP3 epitpoe linked with pentamerization domains
GSWQTWNAKWDQWSNDWNAWRSDWQAWKDDWARLRALLMGGRLLARLNNIDDHINNLNNEIHEIEKMWLFVKEEIKEVKEEIKELARGGSGNANPNANPNANP

SEQ ID NO: 113 - protein, 103 aa, Artificial Sequence
  Plasmodium falciparum NANP3 epitpoe linked with pentamerization domains
GSWDNWNNNWDNWYNNWDNWNNNWDNWNNNWDNLRALLMGGRLLARLNNIDDHINNLNNEIHEIEKMWLFVKEEIKEVKEEIKELARGGSGNANPNANPNANP

SEQ ID NO: 114 - protein, 103 aa, Artificial Sequence
  Plasmodium falciparum NANP3 epitpoe linked with pentamerization domains
GSWNHWDNRWNHWDNRWNHWDNRWNHWDNRWNHLRALLMGGRLLARLNNIDDHINNLNNEIHEIEKMWLFVKEEIKEVKEEIKELARGGSGNANPNANPNANP

SEQ ID NO: 115 - protein, 102 aa, Artificial Sequence
  Plasmodium falciparum NANP3 epitpoe linked with pentamerization domains
GSDEMLRELQETNAALQDVRELLRQQVRQITFLRCLLMGGRLLCRLEELERRLEELERRLEELERAINTVDLELAALRRRLEELARGGSGNANPNANPNANP

SEQ ID NO: 116 - protein, 102 aa, Artificial Sequence
  Plasmodium falciparum NANP3 epitpoe linked with pentamerization domains
GSDEMLRELQETNAALQDVRELLRQQVRQITFLRCLLMGGRIKEEIKEVKEEIKEVKEEIKEVKEEIKEVKEEIKEEIKEVKELARGGSGNANPNANPNANP

SEQ ID NO: 117 - protein, 102 aa, Artificial Sequence
  Plasmodium falciparum NANP3 epitpoe linked with pentamerization domains
GSDNWNNNWDNWYNNWDNWNNNWDNWNNNWDNWNNNWMGGRIKEEIKEVKEEIKEVKEEIKEVKEEIKEVKEEIKEEIKEVKELARGGSGNANPNANPNANP

SEQ ID NO: 118 - protein, 106 aa, Artificial Sequence
  Plasmodium falciparum NANP4 epitpoe linked with pentamerization domains
GSDEMLRELQETNAALQDVRELLRQQVRQITFLRALLMGGRLLARLEELERRLEELERRLEELERAINTVDLELAALRRRLEELARGGSGNANPNANPNANPNANP

SEQ ID NO: 119 - protein, 107 aa, Artificial Sequence
  Plasmodium falciparum NANP4 epitpoe linked with pentamerization domains
GSWQTWNAKWDQWSNDWNAWRSDWQAWKDDWARLRALLMGGRLLARLEELERRLEELERRLEELERAINTVDLELAALRRRLEELARGGSGNANPNANPNANPNANP

SEQ ID NO: 120 - protein, 107 aa, Artificial Sequence
  Plasmodium falciparum NANP4 epitpoe linked with pentamerization domains
GSWDNWNNNWDNWYNNWDNWNNNWDNWNNNWDNLRALLMGGRLLARLEELERRLEELERRLEELERAINTVDLELAALRRRLEELARGGSGNANPNANPNANPNANP

SEQ ID NO: 121 - protein, 107 aa, Artificial Sequence
  Plasmodium falciparum NANP4 epitpoe linked with pentamerization domains
GSWNHWDNRWNHWDNRWNHWDNRWNHWDNRWNHLRALLMGGRLLARLEELERRLEELERRLEELERAINTVDLELAALRRRLEELARGGSGNANPNANPNANPNANP

SEQ ID NO: 122 - protein, 106 aa, Artificial Sequence
  Plasmodium falciparum NANP4 epitpoe linked with pentamerization domains
GSDEMLRELQETNAALQDVRELLRQQVRQITFLRALLMGGRLLARLKEVKEEIKEVKEEIKEVKEEIKEVKEEIKEEIKEVKELARGGSGNANPNANPNANPNANP

SEQ ID NO: 123 - protein, 107 aa, Artificial Sequence
  Plasmodium falciparum NANP4 epitpoe linked with pentamerization domains
GSWQTWNAKWDQWSNDWNAWRSDWQAWKDDWARLRALLMGGRLLARLKEVKEEIKEVKEEIKEVKEEIKEVKEEIKEEIKEVKELARGGSGNANPNANPNANPNANP

SEQ ID NO: 124 - protein, 107 aa, Artificial Sequence
  Plasmodium falciparum NANP4 epitpoe linked with pentamerization domains
GSWDNWNNNWDNWYNNWDNWNNNWDNWNNNWDNLRALLMGGRLLARLKEVKEEIKEVKEEIKEVKEEIKEVKEEIKEEIKEVKELARGGSGNANPNANPNANPNANP

SEQ ID NO: 125 - protein, 107 aa, Artificial Sequence
  Plasmodium falciparum NANP4 epitpoe linked with pentamerization domains
GSWNHWDNRWNHWDNRWNHWDNRWNHWDNRWNHLRALLMGGRLLARLKEVKEEIKEVKEEIKEVKEEIKEVKEEIKEEIKEVKELARGGSGNANPNANPNANPNANP

SEQ ID NO: 126 - protein, 92 aa, Artificial Sequence
  Plasmodium falciparum NANP4 epitpoe linked with pentamerization domains
GSDEMLRELQETNAALQDVRELLRQQVRQITFLRALLMGGRLLARLENLNNEIHEIEKMWLFIKKKEEILARGGSGNANPNANPNANPNANP

SEQ ID NO: 127 - protein, 93 aa, Artificial Sequence
  Plasmodium falciparum NANP4 epitpoe linked with pentamerization domains
GSWQTWNAKWDQWSNDWNAWRSDWQAWKDDWARLRALLMGGRLLARLENLNNEIHEIEKMWLFIKKKEEILARGGSGNANPNANPNANPNANP

SEQ ID NO: 128 - protein, 93 aa, Artificial Sequence
  Plasmodium falciparum NANP4 epitpoe linked with pentamerization domains
GSWDNWNNNWDNWYNNWDNWNNNWDNWNNNWDNLRALLMGGRLLARLENLNNEIHEIEKMWLFIKKKEEILARGGSGNANPNANPNANPNANP

SEQ ID NO: 129 - protein, 93 aa, Artificial Sequence
  Plasmodium falciparum NANP4 epitpoe linked with pentamerization domains
GSWNHWDNRWNHWDNRWNHWDNRWNHWDNRWNHLRALLMGGRLLARLENLNNEIHEIEKMWLFIKKKEEILARGGSGNANPNANPNANPNANP

SEQ ID NO: 130 - protein, 120 aa, Artificial Sequence
  Plasmodium falciparum NANP4 epitpoe linked with pentamerization domains
GSDEMLRELQETNAALQDVRELLRQQVRQITFLRALLMGGRLLARLNNIDDHINNIDDYINNIDDHINNIDDHINNIDDHINNIDDHINNIDDHINNVARGGSGNANPNANPNANPNANP

SEQ ID NO: 131 - protein, 121 aa, Artificial Sequence
  Plasmodium falciparum NANP4 epitpoe linked with pentamerization domains
GSWQTWNAKWDQWSNDWNAWRSDWQAWKDDWARLRALLMGGRLLARLNNIDDHINNIDDYINNIDDHINNIDDHINNIDDHINNIDDHINNIDDHINNVARGGSGNANPNANPNANPNANP

SEQ ID NO: 132 - protein, 121 aa, Artificial Sequence
  Plasmodium falciparum NANP4 epitpoe linked with pentamerization domains
GSWDNWNNNWDNWYNNWDNWNNNWDNWNNNWDNLRALLMGGRLLARLNNIDDHINNIDDYINNIDDHINNIDDHINNIDDHINNIDDHINNIDDHINNVARGGSGNANPNANPNANPNANP

SEQ ID NO: 133 - protein, 121 aa, Artificial Sequence
  Plasmodium falciparum NANP4 epitpoe linked with pentamerization domains
GSWNHWDNRWNHWDNRWNHWDNRWNHWDNRWNHLRALLMGGRLLARLNNIDDHINNIDDYINNIDDHINNIDDHINNIDDHINNIDDHINNIDDHINNVARGGSGNANPNANPNANPNANP

SEQ ID NO: 134 - protein, 106 aa, Artificial Sequence
  Plasmodium falciparum NANP4 epitpoe linked with pentamerization domains
GSDEMLRELQETNAALQDVRELLRQQVRQITFLRALLMGGRLLARLNNIDDHINNLNNEIHEIEKMWLFVKEEIKEVKEEIKELARGGSGNANPNANPNANPNANP

SEQ ID NO: 135 - protein, 107 aa, Artificial Sequence
  Plasmodium falciparum NANP4 epitpoe linked with pentamerization domains
GSWQTWNAKWDQWSNDWNAWRSDWQAWKDDWARLRALLMGGRLLARLNNIDDHINNLNNEIHEIEKMWLFVKEEIKEVKEEIKELARGGSGNANPNANPNANPNANP

SEQ ID NO: 136 - protein, 107 aa, Artificial Sequence
  Plasmodium falciparum NANP4 epitpoe linked with pentamerization domains
GSWDNWNNNWDNWYNNWDNWNNNWDNWNNNWDNLRALLMGGRLLARLNNIDDHINNLNNEIHEIEKMWLFVKEEIKEVKEEIKELARGGSGNANPNANPNANPNANP

SEQ ID NO: 137 - protein, 107 aa, Artificial Sequence
  Plasmodium falciparum NANP4 epitpoe linked with pentamerization domains
GSWNHWDNRWNHWDNRWNHWDNRWNHWDNRWNHLRALLMGGRLLARLNNIDDHINNLNNEIHEIEKMWLFVKEEIKEVKEEIKELARGGSGNANPNANPNANPNANP

SEQ ID NO: 138 - protein, 106 aa, Artificial Sequence
  Plasmodium falciparum NANP4 epitpoe linked with pentamerization domains
GSDEMLRELQETNAALQDVRELLRQQVRQITFLRCLLMGGRLLCRLEELERRLEELERRLEELERAINTVDLELAALRRRLEELARGGSGNANPNANPNANPNANP

SEQ ID NO: 139 - protein, 106 aa, Artificial Sequence
  Plasmodium falciparum NANP4 epitpoe linked with pentamerization domains
GSDEMLRELQETNAALQDVRELLRQQVRQITFLRCLLMGGRIKEEIKEVKEEIKEVKEEIKEVKEEIKEVKEEIKEEIKEVKELARGGSGNANPNANPNANPNANP

SEQ ID NO: 140 - protein, 106 aa, Artificial Sequence
  Plasmodium falciparum NANP4 epitpoe linked with pentamerization domains
GSDNWNNNWDNWYNNWDNWNNNWDNWNNNWDNWNNNWMGGRIKEEIKEVKEEIKEVKEEIKEVKEEIKEVKEEIKEEIKEVKELARGGSGNANPNANPNANPNANP

SEQ ID NO: 141 - protein, 13 aa, Artificial Sequence
  natural PADRE sequence
AKFVAAWTLKAAA

SEQ ID NO: 142 - protein, 13 aa, Artificial Sequence
  Natural PADRE sequence
AKFVAANTLKAAA

SEQ ID NO: 143 - protein, 13 aa, Artificial Sequence
  Natural PADRE sequence
AKFVAAYTLKAAA

SEQ ID NO: 144 - protein, 13 aa, Artificial Sequence
  Natural PADRE sequence
AKFVAAKTLKAAA

SEQ ID NO: 145 - protein, 13 aa, Artificial Sequence
  Natural PADRE Sequence
AKFVAAHTLKAAA

SEQ ID NO: 146 - protein, 13 aa, Artificial Sequence
  Natural PADRE Sequence
AKFVAAATLKAAA

(19) United States
(12) Patent Application Publication
(10) Pub. No.: US 2012/0015000 A1
(43) Pub. Date: Jan. 19, 2012

(54) MALARIA VACCINE OF SELF-ASSEMBLING POLYPEPTIDE NANOPARTICLES

(76) Inventors: David Lanar, Takoma Park, MD (US); Peter Burkhard, Mansfield Center, CT (US)

(21) Appl. No.: 13/056,298

(22) PCT Filed: Jun. 29, 2009
(86) PCT No.: PCT/US09/49131

§ 371 Date: Jan. 27, 2011

Related U.S. Application Data
(60) Provisional application No. 61/076,963, filed on Jun. 30, 2008.

(51) Int. Cl. A61K 9/14; A61P 33/06; A61K 39/015; A61P 37/04; B82Y 5/00

(52) U.S. Cl. 424/400; 424/272.1; 424/191.1; 424/192.1; 977/773; 977/917

(57) ABSTRACT

The invention is directed to functionalized self-assembling polypeptide nanoparticles, and to methods of using these nanoparticles to vaccinate against malaria. The functionalized SAPN comprises a self-assembling core, and at least one epitope fused to the self-assembling core. The self-assembling core comprises a pentameric coiled-coil domain, a trimeric coiled-coil domain, and a linker. The linker joins the pentameric coiled-coil domain and the trimeric coiled-coil domain. Particular sequences of the epitopes used in the vaccine are from the Plasmodium parasite.

### REFERENCE TO RELATED APPLICATIONS

This application claims the benefit of U.S. Provisional Application No. 61/076,963 filed Jun. 30, 2008. The entirety of each of these documents is specifically and entirely incorporated by reference

### RIGHTS IN THE INVENTION

This invention was made with support from the United States Government and, specifically, the Walter Reed Army Institute of Research and, accordingly, the United States government has certain rights in this invention.

### BACKGROUND

1. Technical Field

This invention is directed to self-assembling polypeptide nanoparticles for the diagnosis and treatment of malaria and, in particular, nanoparticles containing specific epitope constructions of antigens derived from malarial proteins.

2. Background

Malaria is caused by protozoan parasites of the genus Plasmodium. At least four types of the plasmodium parasite infect humans, although the most serious forms of the disease are caused by Plasmodium falciparum and Plasmodium vivax. Related species include Plasmodium ovale and Plasmodium malariae, which also infect humans. The group of human-pathogenic Plasmodium species is usually referred to as malaria parasites.

The organism itself is transmitted by the bite of an infected Anopheles mosquito. When an infected mosquito bites a human, sporozoites enter the human circulation. These travel to and penetrate liver cells where they asexually reproduce, via the process of schizogony. The intracellular, asexually dividing form of the parasite is referred to as a schizont, and because this schizont is in liver cells and not red blood cells (RBCs), it is referred to as the exoerythrocytic schizont stage. In Plasmodium vivax and Plasmodium ovale, the development of the schizont is retarded, and a resting stage of the parasite, called the Hypnozoite, is formed; however, this is not the case in Plasmodium falciparum.

When the hepatocytes burst, exoerythrocytic schizonts release merozoites into the blood, which are capable of infecting erythrocytes. Inside the erythrocytes, the merozoites develop into ring-like trophozoites, which then form the erythrocytic schizonts. Mature erythrocytic schizonts form merozoites again by breaking apart inside the erythrocytes. These merozoites are a transient intracellular form, either rapidly infecting new red blood cells to complete the erythrocytic cycle, or dying. In addition, when infection of new blood cells occurs, instead of forming trophozoites the parasites may grow into the immature gametocytes. These can be taken up in the blood meal of another feeding mosquito. The male gametocyte undergoes rapid nuclear division and produces a flagellated microgamete, which fertilizes the female gametocyte forming a zygote. The zygote develops into an ookinete, which then sticks to the gut wall of the mosquito, moves to the outermost layer of the stomach to form an oocyst. When the oocyst breaks, it releases sporozoites, which migrate to the salivary glands of the mosquito to restart the parasite's life cycle.

The disease malaria afflicts 500 million people worldwide and annually kills about 3 million people, most of whom are children. The Walter Reed Army Institute of Research (WRAIR) and Glaxo Smith Kline (GSK) developed the most successful vaccine to date. That vaccine, referred to as “RTS,S”, is based on the circumsporozoite protein (CSP), the most abundant surface protein on the sporozoite, the parasite stage that mosquitoes inject into humans that starts the infection. The RTS,S Virus-Like Particle (VLP) vaccine is comprised of a C-terminal fragment of CSP fused to the Hepatitis B Surface Protein and is synthesized by S. cerevisiae. It requires formulation with the adjuvant AS02A to achieve protective immunogenicity. At best, this vaccine provides only about 40% protective efficacy in human clinical and field challenge studies. Many other malaria vaccines based on the CSP and other malaria proteins have proven unsuccessful [3]. Also, many experimental adjuvants have been tested and shown to produce either insufficient immunogenicity or unacceptable reactogenicity. Furthermore, a variety of antigen presentations, including single recombinant proteins, multi-antigen combinations, malaria fusion protein fragments, or single or multiple peptide epitopes arrays have produced little success in preventing disease.

The development of a vaccine for P. falciparum malaria has been extremely difficult for at least two reasons. The first is that the P. falciparum parasites do not reliably infect animals, although a few non-human primate models are available for blood stage vaccine work, thus making the testing of vaccine designs difficult. For sporozoite vaccine work, therefore, rodent malaria models based on P. berghei or P. yoelii (or P. chabaudi or P. vinckei) are used for preliminary vaccine studies. Because the blood stage of the parasite can be cultured in human erythrocytes, antibodies against blood stage proteins can be tested for their capacity to prevent invasion of erythrocytes by merozoites, but this event has yet to be definitively identified as a correlate of protective immunity. The second is that most malaria epitopes are not very immunogenic in man. It is believed this is the result of thousands of years of evolution of the malaria parasite living in man and evolving epitopes on its functionally important proteins that are not recognized by the human immune system. Therefore, the advances in malaria vaccinology have had to rely on adjuvants to increase the immune response to many malaria proteins in vaccines developed for human use. Most adjuvants used in animal studies have adverse side effects that make them unsafe to use in humans, and while there are several new ones in clinical trials, only alum is currently approved for human use, and alum has proved to be a poor adjuvant for malaria vaccines.

Peptide based vaccines against malaria have been made before but all relied on strong adjuvants for protective efficacy. Mouse studies with the murine malaria parasite P. berghei have shown that vaccines based on immunodominant CSP B- or T cell epitopes can induce a protective immune response if given with strong adjuvants. Analysis of murine immune responses to vaccination with the P. berghei CSP (PbCSP) have shown its dominant B cell epitope to be (DPPPNPND)₂ (SEQ ID NO 1) [4, 5], which, like the PfCSP epitope, is located in the central repeat portion of the protein. A cytotoxic T cell epitope, NDDSYIPSAEKI (SEQ ID NO 2), has also been identified [6]. A synthetic peptide vaccine containing a tandemly repeating domain (DPPPPNPN)₂ (SEQ ID NO 3) has been produced using a “multiple antigenic peptide system” (MAPS), in which the synthetic peptides are linked to a lattice matrix of lysines [7-9]. Later constructs (TB4 and BT4) contained four copies of both the B cell epitope and a T cell epitope KQIRDSITEEWS (SEQ ID NO 4). While these constructs induced a protective (˜80-100%) immune response to sporozoite challenge, the MAPS had to be emulsified with Complete Freund's Adjuvant (CFA) or Incomplete Freund's Adjuvant (IFA) before delivery. When mice were immunized with MAPS adsorbed to alum, the induced antibody titers were only about 25% of titers achieved with CFA delivery. Immunization with the MAPS combined in buffered saline, without adjuvant, elicited only minimal antibody titers and did not induce a protective immune response.

Particulate antigens are generally more immunogenic than non-particulate antigens. In recent years it has been recognized that particulate antigens such as virosomes [10-12], immunostimulating complexes (ISCOMS; [13, 14], PLG microparticles [15], and virus-like particles (VLP) [16-19] generally induce more effective humoral and cellular immune responses than those induced by soluble antigens. The VLP is a subunit vaccine that contains one or a few structural proteins of a virus that self-assemble into highly organized particulate structures. Incorporation of epitopes into the virus protein provides a way to deliver the epitope as an immunogen. The disadvantages to VLP are: 1) preexisting immunity to the virus may inhibit its use as a vaccine; 2) some VLP are large in size and their uptake by the immune system's dendritic cells may be difficult; and, 3) the use of the VLP may cause an undesired or preferential immune response to the VLP proteins which may in turn reduce the desired immune response to the vaccine epitope. Another major disadvantage of VLPs are that they are much less well understood with regard to their flexibility for tolerating modifications without disruption of the capsid-like structures.

Several VLP platforms have been tested in malaria vaccine development. Schodel made recombinant Hepatitis B core antigen (HBcAg) VLP incorporating the immunodominant B cell epitopes for P. falciparum (NANP)₃ (SEQ. ID NO.93), P. berghei (DPPPPNPN)₂, (SEQ ID NO 3) and P. yoelii (SYVPSAEQI) (SEQ ID NO 5) [20, 21]. The resulting hybrid HBcAg-CS proteins were particulate but required CFA, IFA, or alum for immunogenicity. Oliveira-Ferreira [22] put the CD8⁺ cell epitope of the P. yoelii CSP into the yeast VLP from retro-transposon Ty and attempted to immunize mice without adjuvant. The construct (TyCS VLP) was either preceded or followed by a dose of recombinant vaccinia virus expressing the entire P. yoelii CSP (VacPyCS). TyCS VLP or VacPyCS on their own induced undetectable or minimal T cell responses. Only the combination of TyCS VLP followed by VacPyCS was effective in induction of CSP specific CD8⁺ T cells capable of reducing the amount of plasmodial parasites, and at best only 62% of mice challenged were protected. Two immunizing doses of TyCS VLP in PBS had no detectable effect. Plebanski [23] cloned the P. berghei cytotoxic CD4⁺ T cell epitope (SYIPSAEKI) (SEQ ID NO 6) into the Ty vector. Constructs containing one or two epitopes administered intravenously at 100 μg/mouse in PBS induced good CTL responses but could not, on their own, induce a protective response to sporozoite challenge. Only upon heterologous boosting with a vaccinia construct containing the P. berghei CSP epitope was a protective immune response induced. The VLP construct did not have the capacity to boost the immune response by a second or third dose of VLP. Another PfCSP based vaccine, ICC-1134, containing both T- and B-cell PfCSP epitopes in a modified Hepatitis B Virus core particle [24-26] was shown to be immunogenic if mixed with Montanide ISA-720™ (Ste D'exploitation De Produits Pour Les Industries Chimiques-S.E.P.P.I.C. Corporation Quai D'orsay 75321 Paris Cedex 07 France). However, multiple doses produced undesired adverse events in primates, and therefore only a single injection was used in a Phase I/IIa study resulting in minimal immunogenicity and no protection to sporozoite challenge. RTS,S, the P. falciparum CSP based vaccine, is a formulation of a VLP and the PfCSP protein fused with Hepatitis B Surface antigen, mixed with unfused Hepatitus B Surface Antigen in a proprietary combination and formulated with AS02A adjuvant. In multiple clinical trials and two field trials the vaccine has consistently only protected about 40% of vaccines from infection and the protection seems to wane after about 6 months. The vaccine is not a true VLP but more a mixture of about 75% Hep-Surface Protein and a PfCSP-HepB Surface Protein fusion that when mixed forms a particulate antigen.

### Importance of Particle Size

Lymph node uptake: While it was previously thought that lymph nodes contained only mature DC incapable of further processing it has been recently proven that a substantial fraction of DC in the lymph nodes are immature and still capable of internalizing and processing antigen [27-29]. It has been determined that one of the important requirements for lymphatic system uptake from the interstitial space is particle size. Small particles (<40 nm) are quickly and easily taken up by lymphatic vessels [30]. ID injection of 20 nm particles are rapidly and highly efficiently taken up by lymphatic vessels, and retention in lymph nodes lasts for up to 120 h post-injection [31].

Epitope density and Ig cross-linking. It was noted early on in immunology that small organic molecules were not immunogenic, average sized proteins were only a little immunogenic, while protein complexes could elicit a stronger immune response. Larger, well ordered protein assemblies like VLPs [32] belong to the strongest immunogens that are known, especially if they repetitively display an antigenic epitope [33, 34]. The correlation of the size of the immunogen along with the density of the displayed antigen with the strength of the immune response is very difficult to establish. Nevertheless, decades ago Dintzis et al. demonstrated that such a correlation existed and that the spacing between epitopes was critically important for the strength of the immune response [35-37]. The organization of proteins on viral capsid structures increases the immune response significantly as opposed to the single soluble proteins [38]. More recently, Liu and Chen [39] have shown that antibody affinity constants are as much as 2 logs higher when antigens are displayed in optimal density arrays.

Thus there is a need for an inexpensive malaria vaccine that will prevent the death and debilitation of millions annually. Such a vaccine would also be useful widely for the existing populations as well as tourists, visitors, and also government and medical workers, refugees and other displaced people, soldiers and peacekeepers, and others who are deployed on humanitarian missions to malaria endemic areas, particularly Southeast Asia, Africa, as well as Central and South America.

### BRIEF DESCRIPTION

The present invention is directed to self-assembling polypeptide nanoparticles, and to methods of using these nanoparticles for the diagnosis and treatment of malaria.

One embodiment of the present invention is directed to peptidic nanoparticles comprising self-assembling polypeptides which each comprises a pentameric domain and a trimeric domain; a linker which joins the pentameric domain and the trimeric domain; and an epitope comprising a sequence of a malarial antigen fused to the self-assembling polypeptide. Preferably the epitope is a universal epitope and may be fused to the self-assembling core at an exposed terminus which is an N-terminus or a C-terminus, and the epitope is a T-cell epitope or a B-cell epitope. Preferably, nanoparticles each contain one or more antigens as listed in Table 2. Also preferable, the antigen contains a sequences containing one or more of the SEQ ID NOs listed in table 3. Nanoparticles of the invention may further comprise a second epitope, wherein the second epitope is a T-cell epitope or a B-cell epitope.

Nanoparticles are very thermostable and are candidates for vaccines. Nanoparticles are of roughly homogeneous size, and spherical appearance, with a diameter of about 25 nm. Preferably, the nanoparticle self-assembling polypeptide contains no disulfide cross-linking.

Also preferably, an assembly of nanoparticles remains non-aggregated in solution in the absence of a reducing agent over a period of months. Preferred nanoparticles are useful for the treatment and prevention of malaria and the epitope is derived from the P. falciparum sporozoite protein.

Another embodiment of the invention is directed to vaccines for the prevention or treatment of malaria. Vaccines of the invention comprise a self-assembling polypeptide comprising a pentameric domain; a trimeric domain; and a linker that joins the pentameric domain and the trimeric domain; and an epitope of an antigen capable of inducing a protective immune response in a mammal susceptible to infection by a malaria parasite. Preferably the self-assembling polypeptide is a continuous chain comprising peptide oligomerizations of the pentameric domain and the trimeric domain. Vaccines of the invention comprises the antigens and proteins set forth in Table 2 or one or more of the sequences set forth in Table 3. Vaccines preferably contain a pharmaceutically acceptable carrier. Preferred vaccines include a construct containing the circumsporozoite protein antigen of P. falciparum.

Another embodiment of the invention is directed to methods for vaccinating against infection from a malaria parasite. These methods comprise administering a functionalized self-assembling polypeptide nanoparticle comprising a self-assembling core; and an epitope fused to the self-assembling core, wherein the self-assembling core comprises a pentameric coiled-coil domain; a trimeric coiled-coil domain; and a linker joining the pentameric coiled-coil domain and the trimeric coiled-coil domain wherein the epitope generates an immunologically protective reaction against infection by a malaria parasite when administered to a mammal. Preferably the nanoparticle is administered without an adjuvant and the epitope is PfCSP. Also preferably, the epitope is a universal epitope comprising the sequence of SEQ ID NO. 8 or SEQ ID NO. 9.

Another embodiment of the invention is directed to an icosahedral particle comprising functionalized self-assembling polypeptide nanoparticles, wherein each self-assembling polypeptide nanoparticle comprises a self-assembling core, and an epitope fused to the self-assembling core, wherein the self-assembling core comprises a pentameric coiled-coil domain, a trimeric coiled-coil domain, and a linker, said linker joining the pentameric coiled-coil domain and the trimeric coiled-coil domain, and wherein the icosahedral particle is formed by multimerization via the coiled-coil sequences. Particles typically have a diameter of about 25 nm and contain an antigen of a malaria parasite. Preferably the antigen is derived from a protein of P. falciparum such as the circumsporozoite protein.

Other embodiments and advantages of the invention are set forth in part in the description, which follows, and in part, may be obvious from this description, or may be learned from the practice of the invention.

### DESCRIPTIONS OF THE DRAWINGS

FIG. 1. A schematic of a linear self-assembling polypeptide building block of a SAPN vaccine.

FIG. 2 Schematic drawing of “even units” for trimeric and pentameric oligomerization domains [left side, A)] and trimeric and tetrameric oligomerization domains [right side, BA)], respectively. The number of monomers (building blocks) is defined by the least common multiple (LCM) of the oligomerization states of the two oligomerization domains D1 and D2 of the building blocks. In the even units the linker segments of all building blocks will be arranged as closely to each other as possible, i.e. as close to the center of the peptidic nanoparticle as possible and hence the even units will self-assemble to a spherical nanoparticle.

FIG. 3. A model of the SAPN showing: A) a monomer peptide sequence composed of a trimeric coiled-coil, a linker segment, a pentameric coiled-coil and a disulfide bridge; B) the self-assembly of multiple SAPN via trimer and pentamer oligomerization; C) a completely assembled 60 mer icosahedrons SAPN.

FIG. 4. The architecture of a nanoparticles constructed from various elements shown as a figure and as an EM photograph.

FIG. 5. A schematic of the linear, self-assembling polypeptides.

FIG. 6. A bar graph summarizing the survival of mice in two separate experiments totaling 40 mice, 20 C57BL/6 and 20 Balb/c in each group.

FIG. 7. A graph related to parasitemic mice (Balb/c) vaccinated with SAPN with or without adjuvant are provided sterile protection. Groups of 10 mice receiving 3 doses of vaccine were challenged with P. berghei sporozoites. Mice receiving PBS, N-Empty or N-Empty/M all developed parasitemia by day 6. Mice receiving N-PbCSP or NPbCSP/M or were vaccinated with irradiated sporozoite vaccination did not demonstrate detectable parasitemia. Similar results were seen in C57BL/6 mice.

FIG. 8. Antibody response to N-PbCSP in Balb/C mice.

FIG. 9. The percent of mice developing parasitemia after P. berghei sporozoite challenge. Mice (Balb/c) had received either splenocytes or serum for mice that had been previously immunized with nanoparticles expressing the P. berghei B cell epitope (PbCSPr) administered with or without adjuvant. Control mice received no cells or serum.

FIG. 10. A bar graph of IgG Isotype profile of Balb/c and C57BL/6 mice after three immunizations with P. berghei CSP B cell repeat containing nanoparticles.

FIG. 11. The potency of N-PbCSP without adjuvant. Mice were given the indicated μg of protein, in each of 3 doses, 2 wks apart then challenged with sporozoites.

FIG. 12. SAPN made with NCS-PfAMA and NCS-PbCSP in the indicated ratios protected mice from challenge.

FIG. 13. A cloning strategy for producing different sized and differently functionalized SAPN.

FIG. 14. A flow chart of the process of optimizing SAPN constructs.

FIG. 15. From left to right X-ray crystal structures of COMP, the Trp-zipper and a de-novo-designed trimeric coiled-coil corresponding to the pdb-codes 1VGF, 1T8Z and 1KYC, respectively.

FIG. 16. The sequences show the pentamer (bold), the linker region (regular font), the trimer (italicized), the epitope (highlighted) and the restriction sites (underscored). The heptad repeat pattern for the pentamer and the trimer is indicated above the sequences as a and d positions.

FIG. 17. Effect of PADRE addition to nanoparticles that contain Trp zip motif, T81c-Mal.

### DETAILED DESCRIPTION OF THE PREFERRED EMBODIMENTS

The adaptive immune system has two different responses, the humoral immune response and the cellular immune response. The first is characterized by an antibody response in which these antibodies bind to surface epitopes of pathogens while the latter is characterized by cytotoxic T-lymphozytes (CTLs) that kill already infected cells. Both immune responses are further stimulated by T-helper cells that activate either the B-cells that are producing specific pathogen binding antibodies or T-cells that are directed against infected cells.

The specificity of the interaction between the antibodies produced by B-cells and the pathogen is determined by surface structures of the pathogen, so called B-cell epitopes, while the specificity of the interaction of CTLs with the infected target cell is by means of T-cell epitopes presented on surface molecules of the target cell, the so-called major histocompatibility complex class I molecules (MHC I). This type of T-cell epitopes (CTL epitopes) are fragments of the proteins from the pathogen that are produced by the infected cell. Finally, the specificity of the interaction of the T-helper cells with the respective B-cell or CTL is determined by binding of receptor molecules of the T-helper cells to the other type of T-cell epitopes (HTL-epitopes) presented by the MHC class II molecules (MHC II) on the B-cells or CTL-cells.

Binding of the antibodies to the B-cell epitopes requires the B-cell epitope to assume a particular three-dimensional structure, the same structure that this B-cell epitope has in its native environment, i.e. when it is on the surface of the pathogen. The B-cell epitope may be composed of more than one peptide chain and is organized in a three dimensional structure by the scaffold of the protein.

The T-cell epitopes, however, do not require a particular three-dimensional structure, rather they are bound by the respective MHC I or MHC II molecule in a very specific manner. CTL epitopes are trimmed to a size of 9 amino acids in length for optimal presentation by the MHC I molecules, while HTL epitopes make a similar interaction with the MHC II molecules but may be longer than just 9 amino acids. Important in the context of this invention is, that the binding of the epitopes to the MHC molecules follows very particular rules, i.e. only peptides with specific features will be able to bind to the respective MHC molecule and hence be useful as epitopes. These features have been thoroughly investigated and from the wealth of epitopes known, prediction programs have been developed that are able to predict with high accuracy epitopes that are able to bind to the MHC molecules. Peptide strings composed of several such T-cell epitopes in a linear peptide chain are now being engineered as vaccine candidates.

In general an efficient vaccine should induce a strong humoral as well as a strong cellular immune response. In this patent, self-assembling peptide nanoparticles (SAPN) composed of trimeric and pentameric protein oligomerization domains have been engineered that repetitively display B-cell epitopes on their surface. The B-cell epitopes were attached at the end of the oligomerization domains in order to guarantee that the B-cell epitopes are presented at the surface of the nanoparticles in multiple copies. One of the most frequently encountered protein oligomerization motif is the coiled-coil structural motif and this motif can efficiently be used in the design of these SAPN.

Malaria is a debilitating and often fatal disease, yearly affecting millions of people worldwide. Investigations into a possible vaccine have been the subject of intense research, but the challenges faced in developing a useful vaccine against P. falciparum, the most common form of the parasite, have been daunting. Many of the likely candidate malarial antigens that appear to be immunogenic have proved unsuccessful as vaccines in subsequent testing and/or clinical trials. Conventional vaccines have largely been ineffective, impractical or unable to induce a significantly protective immune response.

A peptidic delivery system has been discovered that offers patients a new opportunity for a vaccine against malaria (see US Patent Publication No. US 2007/0014804 which is entirely incorporated by reference). This delivery system comprises a single continuous chain of a small number of peptide domains linked through a linker segment which self assembles into a nanoparticle. To that nanoparticle is coupled antigenic determinants designed to elicit a protective immune response. It has been surprisingly discovered that certain antigenic determinants of the malaria parasite are effective when implemented as a peptidic-nanoparticle vaccine. The preferred embodiments of the invention provide a flexible nanoparticle vaccine, displaying multiple copies of a single B cell epitope, which can be delivered without adjuvant, imparted a high-titer, boostable, sterile protective immune response effective against a lethal sporozoite challenge.

### Nanoparticle Delivery System

Self-assembling peptide nanoparticles (SAPN) are formed from a multitude of monomeric building blocks of formula (I) consisting of a continuous chain comprising a peptidic oligomerization domain D1, a linker segment L and a peptidic oligomerization domain D2:

D1-L-D2 (I),

wherein D1 is a synthetic or natural peptide having a tendency to form oligomers (D1)m of m subunits D1, D2 is a synthetic or natural peptide having a tendency to form oligomers (D2)n of n subunits D2, m and n each is a number between 2 and 10, with the proviso that m is not equal n and not a multiple of n, and n is not a multiple of m, L is a bond or a short linker chain selected from optionally substituted carbon atoms, optionally substituted nitrogen atoms, oxygen atoms, sulfur atoms, and combinations thereof; either D1 or D2 or both D1 and D2 is a coiled-coil that incorporates one or more T-cell epitopes and/or a B cell epitope within the oligomerization domain, and wherein D1, D2 and L are optionally further substituted.

A peptide (or polypeptide) is a chain or sequence of amino acids covalently linked by amide bonds. The peptide may be natural, modified natural, partially synthetic or fully synthetic. Modified natural, partially synthetic or fully synthetic is understood as meaning not occurring in nature. The term amino acid embraces both naturally occurring amino acids selected from the 20 essential natural α-L-amino acids, synthetic amino acids, such as α-D-amino acids, 6-aminohexanoic acid, norleucine, homocysteine, or the like, as well as naturally occurring amino acids which have been modified in some way to alter certain properties such as charge, such as phoshoserine or phosphotyrosine, or the like. In derivatives of amino acids the amino group forming the amide bond is alkylated, or a side chain amino, hydroxy or thio functions is alkylated or acylated, or a side chain carboxy function is amidated or esterified.

A short linker chain L is selected from optionally substituted carbon atoms, optionally substituted nitrogen atoms, oxygen atoms, sulfur atoms, and combinations thereof, with preferably 1 to 60 atoms, in particular 1 to 20 atoms in the chain. Such a short linker chain is, e.g. a polyethylenoxy chain, a sugar chain or, preferably, a peptide chain, e.g. a peptide chain consisting of 1 to 20 amino acids, in particular 1 to 6 amino acids.

m and n each is a number between 2 and 10, with the proviso that m is not equal n and not a multiple of n, and n is not a multiple of m. Preferred combinations of n and m are combinations wherein m is 2 and n is 5, or m is 3 and n is 4 or 5, or m is 4 and n is 5. Likewise preferred combinations of n and m are combinations wherein m is 5 and n is 2,or m is 4 or 5 and n is 3, or m is 5 and n is 4. Most preferred are combinations wherein m or n is 5.

A coiled-coil is a peptide sequence with a contiguous pattern of mainly hydrophobic residues spaced 3 and 4 residues apart, which assembles to form a multimeric bundle of helices, as will explained in more detail hereinbelow.

A “coiled-coil that incorporates T-cell and/or B-cell epitopes” means that the corresponding epitope is comprised within an oligomerization domain such that the amino acid sequences at the N-terminal and the C-terminal ends of the epitope force the epitope to adapt a conformation which is still a coiled-coil in line with the oligomerization properties of the oligomerization domain comprising the epitope. In particular, “incorporated” excludes a case wherein the epitope is attached at either end of the coiled-coil oligomerization domain.

In the context of this document the term T-cell epitopes shall be used to refer to both CTL and HTL epitopes.

Optional substituents of D1, D2 and L include but are not limited to B-cell epitopes, targeting entities, or substituents reinforcing the adjuvant properties of the nanoparticle, such as an immunostimulatory nucleic acid, preferably an oligodeoxynucleotide containing deoxyinosine, an oligodeoxynucleotide containing deoxyuridine, an oligodeoxynucleotide containing a CG motif, or an inosine and cytidine containing nucleic acid molecule. Other substituents reinforcing the adjuvant properties of the nanoparticle are antimicrobial peptides, such as cationic peptides, which are a class of immunostimulatory, positively charged molecules that are able to facilitate and/or improve adaptive immune responses. Optional substituents, e.g. those optional substituents described hereinabove, are preferably connected to suitable amino acids close to the free end of the oligomerization domain D1 and/or D2. On self-assembly of the peptide nanoparticle, such substituents will then be presented at the surface of the SAPN.

In a most preferred embodiment the substituent is another peptide sequence S1 and/or S2 representing a simple extension of the peptide chain D1-L-D2 at either end or at both ends to generate a combined single peptide sequence of any of the forms S1-D1-L-D2, D1-L-D2-S2, or S1-D1-L-D2-S2, wherein S1 and S2 are peptidic substituents as defined hereinbefore and hereinafter. The substituents S1 and/or S2 are said to extend the core sequence D1-L-D2 of the SAPN. Any such peptide sequence S1-D1-L-D2, D1-L-D2-S2, or S1-D1-L-D2-S2 may be expressed in a recombinant protein expression system as one single molecule.

A preferred substituent S1 and/or S2 is a B-cell epitope. Other B-cell epitopes that may be considered include but are not limited toe hapten molecules such as a carbohydrate or nicotine, which are likewise attached to the end of the oligomerization domains D1 and/or D2, and hence will be displayed at the surface of the SAPN.

Obviously it is also possible to attach more than one substituent to the oligomerization domains D1and/or D2. For example, considering the peptide sequence S1-D1-L-D2-S2, another substituent may be covalently attached to it, preferably at a location distant from the linker segment L, either close to the ends of D1 and/or D2, or anywhere in the substituents S1 and/or S2.

It is also possible to attach a substituent to the linker segment L. In such case, upon refolding of the SAPN, the substituent will be located in the inner cavity of the SAPN.

A “tendency to form oligomers” means that such peptides can form oligomers depending on the conditions, e.g. under denaturing conditions they are monomers, while under physiological conditions they may form, for example, trimers. Under predefined conditions they adopt one single oligomerization state, which is needed for nanoparticle formation. However, their oligomerization state may be changed upon changing conditions, e.g. from dimers to trimers upon increasing salt concentration (Burkhard P. et al., Protein Science 2000, 9:2294-2301) or from pentamers to monomers upon decreasing pH.

A building block architecture according to formula (I) is clearly distinct from viral capsid proteins. Viral capsids are composed of either one single protein, which forms oligomers of 60 or a multiple thereof, as e.g. the hepatitis virus B particles (EP 1 262 555, EP 0 201 416), or of more than one protein, which co-assemble to form the viral capsid structure, which can adopt also other geometries apart from icosahedra, depending on the type of virus (Fender P. et al., Nature Biotechnology 1997, 15:52-56). Self-assembling peptide nanoparticles (SAPN) of the present invention are also clearly distinct from viruslike particles, as they (a) are constructed from other than viral capsid proteins and (b) that the cavity in the middle of the nanoparticle is too small to accommodate the DNA/RNA of a whole viral genome.

Peptidic oligomerization domains are well-known (Burkhard P. et al., Trends Cell Biol 2001, 11:82-88). The most simple oligomerization domain is probably the coiled-coil folding motif. This oligomerization motif has been shown to exist as a dimer, trimer, tetramer and pentamer. Some examples are the GCN4 leucine zipper, fibritin, tetrabrachion and COMP, representing dimeric, trimeric, tetrameric and pentameric coiled coils, respectively.

One or both oligomerization domains D1 and D2, independently of each other, are coiled-coil domains.

### Rules for Coiled-Coil Formation

A “coiled-coil” is a peptide sequence with a contiguous pattern of mainly hydrophobic residues spaced 3 and 4 residues apart, usually in a sequence of seven amino acids (heptad repeat) or eleven amino acids (undecad repeat), which assembles (folds) to form a multimeric bundle of helices. Coiled-coils with sequences including some rregular distribution of the 3 and 4 residues spacing are also contemplated. Hydrophobic residues are in particular the hydrophobic amino acids Val, Be, Leu, Met, Tyr, Phe and Trp. Mainly hydrophobic means that at least 50% of the residues must be selected from the mentioned hydrophobic amino acids.

For example, in a preferred monomeric building block of formula (I), D1 and/or D2 is a peptide of any of the formulae:

[aa(a)-aa(b)-aa(c)-aa(d)-aa(e)-aa(f)-aa(g)]X (IIa),

[aa(b)-aa(c)-aa(d)-aa(e)-aa(f)-aa(g)-aa(a)]X (IIb),

[aa(c)-aa(d)-aa(e)-aa(f)-aa(g)-aa(a)-aa(b)]X (IIc),

[aa(d)-aa(e)-aa(f)-aa(g)-aa(a)-aa(b)-aa(c)]X (IId),

[aa(e)-aa(f)-aa(g)-aa(a)-aa(b)-aa(c)-aa(d)]X (IIe),

[aa(f)-aa(g)-aa(a)-aa(b)-aa(c)-aa(d)-aa(e)]X (IIf),

[aa(g)-aa(a)-aa(b)-aa(c)-aa(d)-aa(e)-aa(f)]X (IIg),

wherein aa means an amino acid or a derivative thereof, aa(a), aa(b), aa(c), aa(d), aa(e), aa(f), and aa(g) are the same or different amino acids or derivatives thereof, preferably aa(a) and aa(d) are the same or different hydrophobic amino acids or derivatives thereof; and X is a figure between 2 and 20, preferably 3, 4, 5 or 6.

Hydrophobic amino acids are Val, Be, Leu, Met, Tyr, Phe and Trp.

A heptad is a heptapeptide of the formula aa(a)-aa(b)-aa(c)-aa(d)-aa(e)-aa(f)-aa(g) (IIa) or any of its permutations of formulae (IIb) to (IIg).

Preferred are monomeric building blocks of formula (I) wherein one or both peptidic oligomerization domains D1 or D2 are:

(1) a peptide of any of the formulae (IIa) to (IIg) wherein X is 3, and aa(a) and aa(d) are selected from the 20 natural α-L-amino acids such that the sum of scores from Table 8 for these 6 amino acids is at least 14, and such peptides comprising up to 17 further heptad; or,

(2) a peptide of any of the formulae (IIa) to (IIg) wherein X is 3, and aa(a) and aa(d) are selected from the 20 natural α-L-amino acids such that the sum of scores from Table 8 for these 6 amino acids is at least 12, with the proviso that one amino acid aa(a) is a charged amino acid able to form an inter-helical salt bridge to an amino acid aa(d) or aa(g) of a neighboring heptad, or that one amino acid aa(d) is a charged amino acid able to form an inter-helical salt bridge to an amino acid aa(a) or aa(e) of a neighboring heptad, and such peptides comprising up to two further heptads. A charged amino acid able to form an interhelical salt bridge to an amino acid of a neighboring heptad is, for example, Asp or Glu if the other amino acid is Lys, Arg or His, or vice versa.

TABLE 8
Scores of amino acid for determination of preference

| Amino Acid | Position aa(a) | Position aa(d) |
|---|---|---|
| L (Leu) | 3.5 | 3.8 |
| M (Met) | 3.4 | 3.2 |
| I (Ile) | 3.9 | 3.0 |
| Y (Tyr) | 2.1 | 1.4 |
| F (Phe) | 3.0 | 1.2 |
| V (Val) | 4.1 | 1.1 |
| Q (Gln) | −0.1 | 0.5 |
| A (Ala) | 0.0 | 0.0 |
| W (Trp) | 0.8 | −0.1 |
| N (Asn) | 0.9 | −0.6 |
| H (His) | −1.2 | −0.8 |
| T (Thr) | 0.2 | −1.2 |
| K (Lys) | −0.4 | −1.8 |
| S (Ser) | −1.3 | −1.8 |
| D (Asp) | −2.5 | −1.8 |
| E (Glu) | −2.0 | −2.7 |
| R (Arg) | −0.8 | −2.9 |
| G (Gly) | −2.5 | −3.6 |
| P (Pro) | −3.0 | −3.0 |
| C (Cys) | 0.2 | −1.2 |

Also preferred are monomeric building blocks of formula (I) wherein one or both peptidic oligomerization domains D1 or D2 are selected from the following preferred peptides:

(11) Peptide of any of the formulae (IIa) to (IIg) wherein aa(a) is selected from Val, Ile, Leu and Met, and a derivative thereof, and aa(d) is selected from Leu, Met and Ile, and a derivative thereof.

(12) Peptide of any of the formulae (IIa) to (IIg) wherein one aa(a) is Asn and the other aa(a) are selected from Asn, Ile and Leu, and aa(d) is Leu. Such a peptide is usually a dimerization domain (m or n=2).

(13) Peptide of any of the formulae (IIa) to (IIg) wherein aa(a) and aa(d) are both Leu or both Be. Such a peptide is usually a trimerization domain (m or n=3).

(14) Peptide of any of the formulae (IIa) to (IIg) wherein aa(a) and aa(d) are both Trp. Such a peptide is usually a pentamerization domain (m or n=5).

(15) Peptide of any of the formulae (IIa) to (IIg) wherein aa(a) and aa(d) are both Phe. Such a peptide is usually a pentamerization or tetramerization domain (m or n=4 or 5).

(16) Peptide of any of the formulae (IIa) to (IIg) wherein aa(a) and aa(d) are both either Trp or Phe. Such a peptide is usually a pentamerization domain (m or n=5).

(17) Peptide of any of the formulae (IIa) to (IIg) wherein aa(a) is either Leu or Ile, and one aa(d) is Gln and the other aa(d) are selected from Gln, Leu and Met. Such a peptide has the potential to be a pentamerization domain (m or n=5).

Other preferred peptides are peptides (1), (2), (11), (12), (13), (14), (15), (16) and (17) as defined hereinbefore, and wherein further:

(21) at least one aa(g) is selected from Asp and Glu and aa(e) in a following heptad is Lys, Arg or His; and/or

(22) at least one aa(g) is selected from Lys, Arg and His, and aa(e) in a following heptad is Asp or Glu, and/or

(23) at least one aa(a to g) is selected from Lys, Arg and His, and an aa(a to g) 3 or 4 amino acids apart in the sequence is Asp or Glu. Such pairs of amino acids aa(a to g) are, for example aa(b) and aa(e) or aa(f).

### Principles of Auto-Assembly

To generate self-assembling peptide nanoparticles (SAPN) with a regular geometry (dodecahedron, cube), more than one even unit is needed. E.g. to form a dodecahedron from a monomer containing trimeric and pentameric oligomerization domains, 4 even units, each composed of 15 monomeric building blocks are needed, i.e. the peptidic nanoparticle with regular geometry will be composed of 60 monomeric building blocks. The combinations of the oligomerization states of the two oligomerization domains needed and the number of even units to form any of the regular polyhedra are listed in Table 1.

TABLE 1
Possible Combinations of Oligomerization States

| | | | | | No. | No. |
|---|---|---|---|---|---|---|
| | | | | | Even | Building |
| Id No. | m | n | Polyhedron Type | LCM | Units | Blocks |
| 1 | 5 | 2 | Dodecahedron/icosahedrons | 10 | 6 | 60 |
| 2 | 5 | 3 | Dodecahedron/icosahedrons | 15 | 4 | 60 |
| 3 | 4 | 3 | Cube/octahedron | 12 | 2 | 24 |
| 4 | 3 | 4 | Cube/octahedron | 12 | 2 | 24 |
| 5 | 3 | 5 | Dodecahedron/icosahedrons | 15 | 4 | 60 |
| 6 | 2 | 5 | Dodecahedron/icosahedrons | 10 | 6 | 60 |
| 7 | 5 | 4 | Irregular | 20 | 1 | 20 |
| 8 | 4 | 5 | Irregular | 20 | 1 | 20 |

Whether the even units will further assemble to form regular polyhedra composed of more than one even unit depends on the geometrical alignment of the two oligomerizations domains D1 and D2 with respect to each other, especially on the angle between the rotational symmetry axes of the two oligomerization domains. This is governed by i) the interactions at the interface between neighboring domains in a nanoparticle, ii) the length of the linker segment L, iii) the shape of the individual oligomerization domains. This angle is larger in the even units compared to the arrangement in a regular polyhedron. Also this angle is not identical in monomeric building blocks as opposed to the regular polyhedron. If this angle is restricted to the smaller values of the regular polyhedron (by means of hydrophobic, hydrophilic or ionic interactions, or a covalent disulfide bridge) and the linker segment L is short enough, a given number of topologically closed even units each containing a defined number of monomeric building blocks will then further anneal to form a regular polyhedron (Table 1) or enclose more monomeric building blocks to from nanoparticles lacking strict internal symmetry of a polyhedron.

If the angle between the two oligomerization domains is sufficiently small (even smaller than in a regular polyhedron with icosahedral symmetry), then a large number (several hundred) peptide chains can assemble into a peptidic nanoparticle. This can be achieved by replacing the two cysteine residues that are located at the interface between the two helices as in the original design of Raman S. et al., Nanomedicine: Nanotechnology, Biology, and Medicine 2006, 2:95-102, and that are forming a disulfide bridge between the two helices, by the small residue alanine. The angle between the two helices can get smaller and consequently more than 60 peptide chains can assemble into a SAPN.

### SAPN as a Next Generation Vaccine Platform

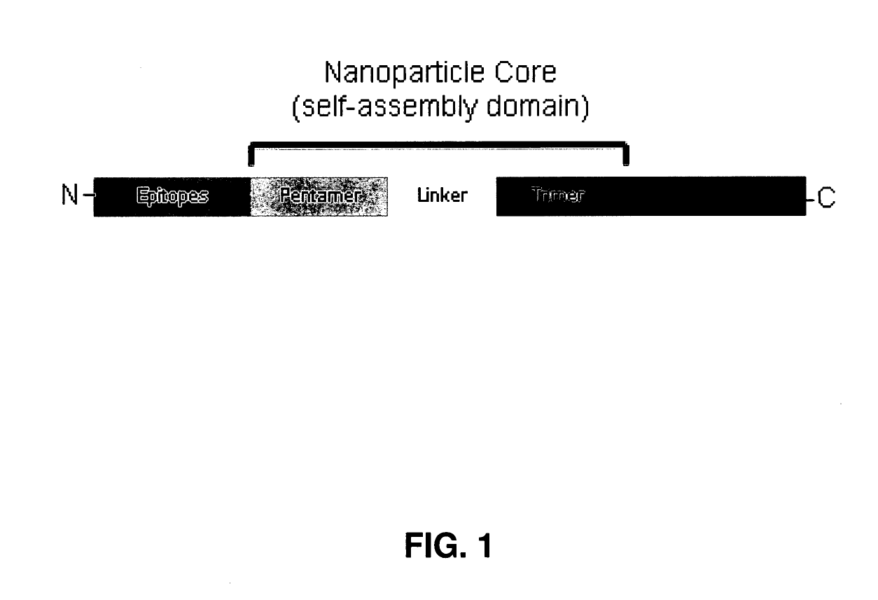

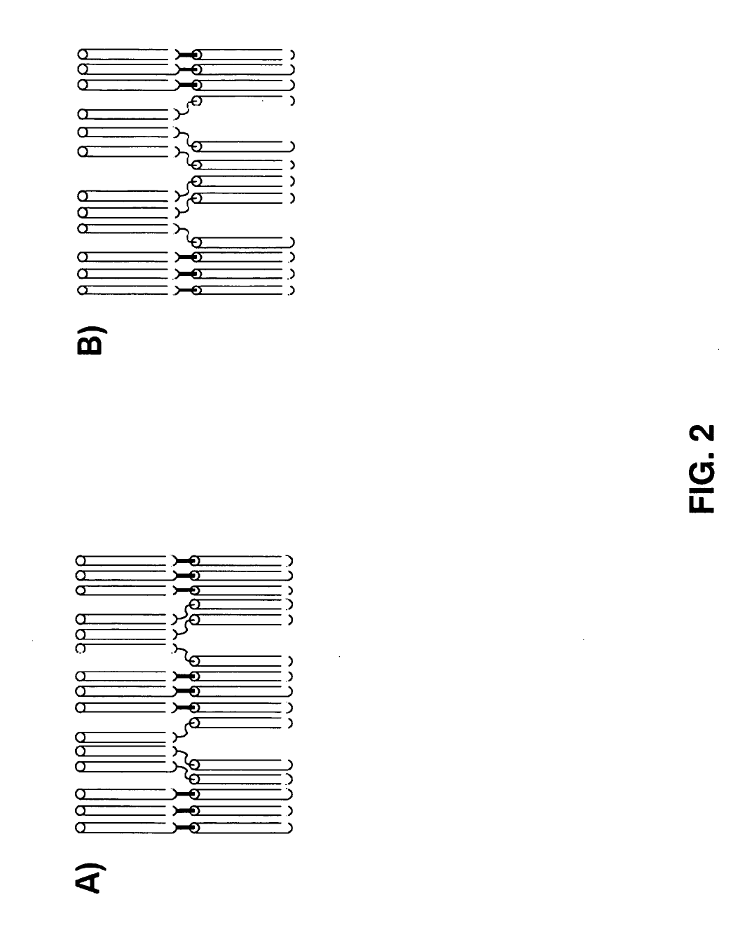

The number of monomeric linear polypeptide building blocks (FIG. 1), which self assemble into a “unit structure” (FIG. 2), is defined by the least common multiple of the two oligomerization domains. A preferred SAPN of the invention has a pentameric and a trimeric oligomerization domain, and thus a unit structure consisting of 15 linear monomeric polypeptides (FIG. 2). The nanoparticle with regular geometry is composed of 4 unit structures or 60 monomeric linear polypetides that have 120 ends per nanoparticle.

Self-assembling peptide nanoparticles (SAPN) are formed from monomeric building blocks of formula (I). If such building blocks assemble, they will form so-called “even units”. The number of monomeric building blocks, which will assemble into such an even unit will be defined by the least common multiple (LCM). Hence, if for example the oligomerization domains of the monomeric building block form a trimer (D1)3 (m=3) and a pentamer (D2)₅ (n=5), 15 monomers will form an even unit (FIG. 2A). If the linker segment L has the appropriate length, this even unit may assemble in the form of a spherical peptidic nanoparticle. Similarly, if the oligomerization domains D1 and D2 of the monomeric building block form a trimer (D1)₃ (m=3) and a tetramer (D2)₄ (n=4), the number of monomers needed to form an even unit will be 12 (FIG. 2B). Since m and n cannot be equal or a multiple of each other, the least common multiple (LCM) is always larger than m and n.

There exist five regular polyhedra (discussed supra), the tetrahedron, the cube, the octahedron, the dodecahedron and the icosahedron. They have different internal rotational symmetry elements. It is sufficient to align the two oligomerization domains D1 and D2 along two of the symmetry axes of the polyhedral formed. If these two oligomerization domains form stable oligomers, the symmetry interface along the third symmetry axis will be generated automatically, and it may be stabilized by optimizing interactions along this interface, e.g. hydrophobic, hydrophilic or ionic interactions, or covalent bonds such as disulfide bridges.

A preferred expression plasmid of the invention is designed for producing the linear self-assembly polypeptides so that each cassette is separated by a restriction site. This permits rapid construction of expression plasmids to enable the testing of a large variety of SAPN, designed based on careful bioinformatic analyses. The single polypeptide chains that comprise the monomeric building blocks are produced in E. coli and purified under denaturing conditions. As the denaturing agent is slowly removed, the monomers self-assemble. Studies show that the peptide building blocks are produced in E. coli at high yield (50 mgs per liter, unoptimized) and that SAPN assembly goes to near completion.

### Molecular Design of SAPN

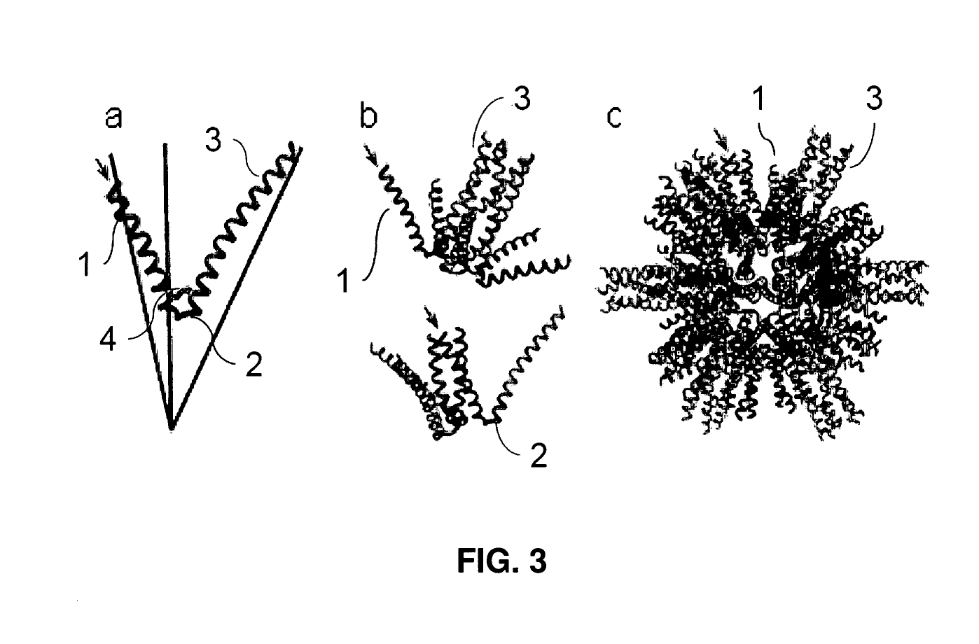

The assembly of the linear polypeptide building blocks into a regular icosahedron depends largely on (i) the interactions at the interface between the trimeric and pentameric oligomerization domains, (ii) the length of the linker segment, and (iii) the shape of the individual oligomerization domains. The testing of different linker constructs has resulted in a linker segment that avoids disruption of the coiled-coil domains and keeps the coiled-coils in close proximity. Turning to FIG. 3, we see examples of a single SAPN monomer (3a), the oligomerization of several SAPN monomers in the beginning stages of assembly (3b) and the final polyhedral SAPN oligomerized polymer at the completion of assembly (3c). FIG. 3a, b and c all show the orientation of the trimeric coiled-coil element 1, the linker segment 2, the pentameric coiled-coil element 3 and a stabilizing chemical bond 4.

To summarize, the formation of SAPN results from at least three types of molecular interactions: 1) the trimeric coiled-coil formation, 2) the pentameric coiled-coil formation, and 3) the interaction between the pentamer and the trimer, which is restricted to a relatively short linker region between them. Furthermore, the pentamer and the trimer form independent units that do not interact with other parts of the SAPN. This indicates that the trimeric and pentameric coiled-coil domains can be replaced by other coiled-coil sequences without abrogating nanoparticle formation—the only condition being that the oligomerization state of the coiled-coils is not changed.

### T-Cell Epitopes and B-Cell Epitopes

Since the T-cell epitopes—as opposed to the B-cell epitopes—do not need to be displayed on the surface of a carrier to cause immunization, they can be incorporated into the core scaffold of the SAPN, i.e. the coiled-coil sequence of an oligomerization domain.

In the present invention it is shown how the features of MHC binding of T-cell epitopes, which requires an extended conformation for MHC binding can be combined with the features of coiled-coil formation, which requires a-helical conformation for coiled coil formation, such that these epitopes can be both, part of the coiled-coil scaffold of the SAPN as well as being able to bind to the respective MHC molecules. It should be noted that not all coiled-coil sequences will be able to bind to MHC molecules and not all T-cell epitopes can be incorporated into a coiled-coil structure.

In a further aspect of this invention B-cell epitopes that are not coiled-coils are incorporated into the coiled-coil sequence of the SAPN oligomerization domain by inserting them between two stretches of coiled-coil segments, such that this whole sequence acts as a single oligomerization domain. This is of particular interest as the coiled-coil scaffold can provide means to restrict the conformation of the B-cell epitope to a conformation that is nearly identical to its native conformation.

### Sources of T-Cell Epitopes

To incorporate T-cell epitopes into an oligomerization domain leading finally to a self-assembling peptide nanoparticle (SAPN), the T-cell epitopes can be chosen from different sources. By way of nonlimiting example, the T-cell epitopes can be determined by experimental methods, they are known from literature, they can be predicted by prediction algorithms based on existing protein sequences of a particular pathogen, or they may be de novo designed peptides or a combination of them.

It is well known that incorporation of HTL epitopes into an otherwise not immunogenic peptide sequence or attaching it to a non-peptidic antigen can make those much more immunogenic. The PanDR binding peptide HTL epitope PADRE has widely been used in vaccine design for a malaria, Alzheimer and many other vaccines and disclosed in U.S. Patent Application Publication No. 20050049197 to Sette et al as incorporated by reference in the entirety herein. Preferred pan DR peptides include: AKFVAAWTLKAAA (SEQ ID NO 141); AKFVAANTLKAAA (SEQ ID NO 142); AKFVAAYTLKAAA (SEQ ID NO 143); AKFVAAKTLKAAA (SEQ ID NO 144); AKFVAAHTLKAAA (SEQ ID NO 145); and AKFVAAATLKAAA (SEQ ID NO 146).

Suitable T-cell epitopes can also be obtained by using prediction algorithms. These prediction algorithms can either scan an existing protein sequence from a pathogen for putative T-cell epitopes, or they can predict, whether de novo designed peptides bind to a particular MHC molecule. Many such prediction algorithms are commonly accessible on the internet. Examples are SVRMHCdb (http://svrmhc.umn.edu/SVRMHCdb; J. Wan et 25 al., BMC Bioinformatics 2006, 7:463), SYFPEITHI (http://www.syfpeithi.de), MHCPred (http://www.jenner.ac.uk/MHCPred), motif scanner (http://hcv.1an1.gov/content/immuno/motif_scan/motif_scan) or NetMHCIIpan (http://www.cbs.dtu.dk/services/NetMHCIIpan) for MHC II binding molecules and NetMHCpan (http://www.cbs.dtu.dk/services/NetMHCpan) for MHC I binding epitopes.

HTL epitopes as described herein and preferred for the design are peptide sequences that are either measured by biophysical methods or predicted by NetMHCIIpan to bind to any of the MHC II molecules with binding affinities (IC50 values) better than 500 nM. These are considered weak binders. Preferentially these epitopes are measured by biophysical methods or predicted by NetMHCIIpan to bind to the MHC II molecules with IC50 values better than 50 nM. These are considered strong binders.

CTL epitopes as described herein and preferred for the design are peptide sequences that are either measured by biophysical methods or predicted by NetMHCpan to bind to any of the MHC I molecules with binding affinities (IC50 values) better than 500 nM. These are considered weak binders. Preferentially these epitopes are measured by biophysical methods or predicted by NetMHCpan to bind to the MHC I molecules with IC50 values better than 50 nM. These are considered strong binders.

### Places for T-Cell Epitopes

The T-cell epitopes can be incorporated at several places within the peptide sequence of the coiled-coil oligomerization domains D1 and or D2. To achieve this, the particular sequence with the T-cell epitope has to obey the rules for coiled-coil formation as well as the rules for MHC binding. The rules for coiled-coil formation have been outlined in detail above. The rules for binding to MHC molecules are incorporated into the MHC binding prediction programs that use sophisticated algorithms to predict MHC binding peptides.

Engineering T-Cell Epitopes into Coiled-Coil

To engineer SAPN that incorporate T-cell epitopes in the coiled-coil oligomerization domain of the SAPN, three steps have to be taken. In a first step a candidate T-cell epitope has to be chosen by using known T-cell epitopes from the literature or from databases or predicted T-cell epitopes by using a suitable epitope prediction program. In a second step a proteasomal cleavage site has to be inserted at the C-terminal end of the CTL epitopes. This can be done by using the prediction program for proteasomal cleavage sites PAProc (http://www.paproc2.de/paproc1/paproc1.html; Hadeler K. P. et al., Math. Biosci. 2004, 188:63-79) and modifying the residues immediately following the desired cleavage site. This second step is not required for HTL epitopes. In the third and most important step the sequence of the T-cell epitope has to be aligned with the coiled-coil sequence such that it is best compatible with the rules for coiled-coil formation as outlined above. Whether the sequence with the incorporated T-cell epitope will indeed form a coiled-coil can be predicted, and the best alignment between the sequence of the T-cell epitope and the sequence of the coiled-coil repeat can be optimized by using coiled-coil prediction programs such as COILS (http://www.ch.embnet.org/software/COILS_form.html; Gruber M. et al., J. Struct. Biol. 2006, 155(2):140-5) or MULTICOIL (http://groups.csail.mit.edu/cb/multicoil/cgi-bin/multicoil.cgi), which are available on the internet.

Even if it is not possible to find a suitable alignment—maybe because the T-cell epitope contains a glycine or even a proline which is not compatible with a coiled-coil structure—the T-cell epitope may be incorporated into the oligomerization domain. In this case the T-cell epitope has to be flanked by strong coiled-coil forming sequences of the same oligomerization state. This will either stabilize the coiled-coil structure to a sufficient extent or alternatively it can generate a loop structure within this coiled-coil oligomerization domain. This is essentially the same procedure as described in the next section for the incorporation of B-cell epitopes into the coiled-coil core sequence of the SAPN.

Engineering B-Cell Epitopes into the Coiled-Coil Core

In a particular aspect of this invention the incorporation into the coiled-coil core of the SAPN of small B-cell epitopes that are not a-helical is envisaged. This can be accomplished by the same procedure as outlined above for the T-cell epitopes that are not compatible with a coiled-coil structure. A B-cell epitope that has an anti-parallel beta-turn conformation can now be incorporated into the coiled-coil core of the SAPN. The coiled-coil structure has to be sufficiently stable to allow incorporation of such a loops structure, hence it must be able to form coiled-coils on both sides of the loop.

### Preferred Design

To engineer a SAPN with the best immunological profile for a given particular application the following consideration have to be taken into account:

CTL epitopes require a proteasomal cleavage site at their C-terminal end. The epitopes should not be similar to human sequences to avoid autoimmune responses—except when it is the goal to elicit an immune response against a human peptide. Accordingly a SAPN is preferred wherein at least one of the T-cell epitopes is a CTL epitope, and, in particular, wherein the sequence further contains a proteasomal cleavage site after the CTL epitope.

Likewise preferred is a SAPN wherein at least one of the T-cell epitopes is a HTL epitope, in particular, a pan-DR-binding HTL epitope. Such pan-DR-binding HTL epitopes bind to many MHC class II and are therefore recognized in a majority of healthy individuals, which is critical for a good vaccine.

Also preferred is a SAPN wherein the sequence D1-L-D2 contains a series of overlapping T-cell epitopes.

B-cell epitopes need to be displayed at the surface of the SAPN. They may or may not be part of the coiled-coil sequence, i.e. the coiled-coil itself may partially be a B-cell epitope depending on whether the portion of the coiled-coil is surface accessible. Coiled-coils of any oligomerization state in general are exceptionally well-suited to be presented in conformation specific manner by the SAPN. Coiled-coils are abundant in the genome of the malaria pathogen Plasmodium falciparum (Villard V. et al., PLoS ONE 2007; 2(7):e645).

### General Considerations for the Design of a Vaccine Against a Pathogen

Such a vaccine preferably contains all three types of epitopes, B-cell, HTL and CTL epitopes. (1) Preferably only one (or very few) B-cell epitope should be placed at either end of the peptide chains. This will place the B-cell epitope on the surface of the SAPN in a repetitive antigen display. (2) The HTL epitopes should be as promiscuous as possible. They do not necessarily need to be derived from the pathogen but can be peptides that elicit a strong T-help immune response. An example would be the PADRE peptide. Preferably these are the T-cell epitopes that are incorporated into the D1-L-D2 core sequence of the SAPN. (3) The CTL epitopes need to be pathogen specific, they need to have C-terminal proteasomal cleavage sites. Since the T-cell epitopes do not require repetitive antigen display several different T-cell epitopes can be incorporated into one single SAPN by co-assembly of different peptide chains that all have the same nanoparticle forming D1-L-D2 core but carry different T-cell epitopes that are not part of the core forming sequence and hence would not be incorporated into the coiled-coil sequences.

### SAPN Engineered Against Malaria

In a preferred aspect of the invention, a composition for the prevention and treatment of malaria is envisaged. Possible protein and peptide sequences suitable for the design of a peptide vaccine may include but are not limited to sequences from the following Plasmodium proteins: MSP-1 (a large polymorphic protein expressed on the parasite cell surface), MSA1 (major merozoite surface antigen 1), CS protein (native circumsporozoite), 35 KD protein or 55 KD protein or 195 KD protein according to U.S. Pat. No. 4,735,799, AMA-1 (apical membrane antigen 15 1), or LSA (liver stage antigen).

Preferred P. falciparum coiled-coil B-cell epitopes are known in the art (Villard V. et al., PLoS ONE 2007, 2(7):e645 and Agak G. W., Vaccine (2008) 26, 1963-1971) and incorporated herein by reference in their entirety. Since for B-cell epitopes only the surface accessible residues are of critical importance for their interactions with the B-cell receptor and the production of antibodies, the coiled-coil core residues at aa(a) and aa(d) positions, which are not surface exposed can be modified to some extent without changing the ability of the immunogen to elicit neutralizing antibodies.

For example, exchanging a valine at an aa(a) postion with an isoleucine will not affect the general immunological properties of the coiled-coil B-cell epitope. Therefore these coiled-coil sequences can be artificially stabilized by optimizing the core residues for best coiled-coil formation and stability without abolishing their immunological potential. Accordingly, modifications of these peptide B-cell epitopes at one or more of their core residues at aa(a) and/or aa(d) in line with the coiled-coil forming propensities as outlined in detail above are also envisioned for these B-cell epitopes.

Preferred P. falciparum CTL epitopes are known as disclosed in U.S. Pat. Nos. 5,028,425, 5 5,972,351, 6,663,871 which are hereby incorporated by reference in their entirety.

One alternate preferred embodiment of the invention provides a malaria vaccine based on a new vaccine platform technology: self-assembling polypeptide nanoparticles (SAPN) that display a high density of B cell epitopes on their surface and T cell epitopes for optimum immunogenicity. These SAPN are assembled from single polypeptides, each comprising a self-assembling core comprising a pentameric and a trimeric coiled-coil domain joined by a linker, with epitopes fused to the N- and/or C-termini. Such a preferred embodiment also includes a process for making peptidic nanoparticles and functionalized peptidic nanoparticles, and monomeric building blocks suitable for forming such nanoparticles. The vaccine preferably contains self-assembling polypeptide nanoparticles displaying the Plasmodium berghei CSP immunodominant B cell epitope (analogous to the P. falciparum CSP B cell epitope in RTS,S) to stimulate a sterile protective immune response against a lethal sporozoite challenge in the P. berghei model and often without the need for an adjuvant.

It was surprisingly discovered that such nanoparticles displaying the Plasmodium berghei CSP immunodominant B cell epitope (analogous to the P. falciparum CSP B cell epitope in RTS,S) stimulate a sterile protective immune response against a lethal sporozoite challenge in the P. berghei model without the need for an adjuvant. This degree of immunogenicity without an adjuvant indicates that the SAPN platform is potentially superior to VLP technologies used to date for the display of malaria antigens, as these have all required adjuvant to be protective. Characteristic of the SAPN vaccine platform malaria vaccine: 1) a SAPN displaying the immunodominant CSP B cell epitope of the mouse malaria, P. berghei (SAPN-PbCSP), completely protected mice from a lethal sporozoite challenge; 2) adjuvant was not required, but when included, increased the rate of the immune response; and, 3) second and third immunizations effectively boosted the immune response.

Antigenic molecules of malaria parasites. As discussed supra, there are many antigenic molecules associated with the malaria parasite as well as antigens that are associated with the mosquito and antigens that are induced upon infection. A non-exhaustive list of antigens that have a high likelihood of usefulness as vaccine targets is listed in Table 2.

TABLE 2

circumsporozoite protein (CSP)
merozoite surface protein
external erythrocyte membrane protein (PfEMP1)
thrombospondin related adhesive protein (TRAP)
apical merozoite antigen (AMA)
liver stage antigen-1 (LSA-1)

Peptide sequences of likely vaccine targets of malaria parasites. Of the antigenic molecules associated with infection, many peptide sequences have been identified that are useful in a nanoparticle vaccine of the invention. A non-exhaustive list of these peptide sequences are listed in Table 3 and Table 7.

TABLE 3

| SEQ ID NOs. | Sequence |
|---|---|
| SEQ ID NO 1 | (DPPPNPND)₂ |
| SEQ ID NO 2 | NDDSYIPSAEKI |
| SEQ ID NO 3 | (DPPPPNPN)₂ |
| SEQ ID NO 4 | KQIRDSITEEWS |
| SEQ ID NO 5 | SYVPSAEQI |
| SEQ ID NO 6 | SYIPSAEKI |
| SEQ ID NO 7 | (DPPPPNPN)₂D |
| SEQ ID NO 8 | EYLNKIQNSLSTEWSPCSVT |
| SEQ ID NO 9 | (DPNANPNV)₂ |
| SEQ ID NO 11 | (DPPPNDVP)₂D |
| SEQ ID NO 12 | KIYNRNTVNRLLAD |
| SEQ ID NO 13 | DPPPPNPN |
| SEQ ID NO 14 | SYPSAEKI |
| SEQ ID NO 15 | NNFDNYNNNCDNYYNNFDNYNNNFDNYNNNFDNYNNNFDNYNNN |
| SEQ ID NO 16 | HNHYDNRYNHHDNRYNHHDNRYNHHDNRYNHHDNRYNHHDNRYNNK |
| SEQ ID NO 18 | GSDEMLRELQETNAALQDVRELLRQQVRQITFLKCLLMGGRLLCRLEELERRLEELE |
| | RRLEELERA |
| SEQ ID NO 19 | GSDEMLRELQETNAALQDVRELLRQQVRQITFLKCLLMGGRLLCRLEELERRLEELE |
| | RRLEELERAINTVDLELAALRRRLEELARGGSGDPPPPNPNDPPPPNPND |
| SEQ ID NO 20 | GSDEMLRELQETNAALQDVRELLRQQVRQITFLKCLLMGGRLLCRLEELERRLEELE |
| | RRLEELERARGGIPSTAFTDIAWVRLPNHY |
| SEQ ID NO. 23 | LERAINTVDLELAALRRRLEELARGGSGDPPPPNPNDPPPPNPND |
| SEQ ID NO. 24 | LERAISAIKADLSALKANLASLQADINTVDLELAALRRRLEELARGGSGDPPPPNPND |
| | PPPPNPND |
| SEQ ID NO. 25 | GSDEMLRELQETNAALQDVRELLRQQVRQITFLRALLMGGRLLARLEELERRLEELE |
| | RRLEELERAINTVDLELAALRRRLEELARGGSGDPPPPNPNDPPPPNPND |
| SEQ ID NO. 26 | GSWQTWNAKWDQWSNDWNAWRSDWQAWKDDWARLRALLMGGRLLARLEELE |
| | RRLEELERRLEELERAINTVDLELAALRRRLEELARGGSGDPPPPNPNDPPPPNPND |
| SEQ ID NO. 27 | GSWDNWNNNWDNWYNNWDNWNNNWDNWNNNWDNLRALLMGGRLLARLEEL |
| | ERRLEELERRLEELERAINTVDLELAALRRRLEELARGGSGDPPPPNPNDPPPPNPND |
| SEQ ID NO. 28 | GSWNHWDNRWNHWDNRWNHWDNRWNHWDNRWNHLRALLMGGRLLARLEELE |
| | RRLEELERRLEELERAINTVDLELAALRRRLEELARGGSGDPPPPNPNDPPPPNPND |
| SEQ ID NO. 29 | GSDEMLRELQETNAALQDVRELLRQQVRQITFLRALLMGGRLLARLKEVKEEIKEV |
| | KEEIKEVKEEIKEVKEEIKEEIKEVKELARGGSGDPPPPNPNDPPPPNPND |
| SEQ ID NO. 30 | GSWQTWNAKWDQWSNDWNAWRSDWQAWKDDWARLRALLMGGRLLARLKEVK |
| | EEIKEVKEEIKEVKEEIKEVKEEIKEEIKEVKELARGGSGDPPPPNPNDPPPPNPND |
| SEQ ID NO. 31 | GSWDNWNNNWDNWYNNWDNWNNNWDNWNNNWDNLRALLMGGRLLARLKEV |
| | KEEIKEVKEEIKEVKEEIKEVKEEIKEEIKEVKELARGGSGDPPPPNPNDPPPPNPND |
| SEQ ID NO. 32 | GSWNHWDNRWNHWDNRWNHWDNRWNHWDNRWNHLRALLMGGRLLARLKEV |
| | KEEIKEVKEEIKEVKEEIKEVKEEIKEEIKEVKELARGGSGDPPPPNPNDPPPPNPND |
| SEQ ID NO. 33 | GSDEMLRELQETNAALQDVRELLRQQVRQITFLRALLMGGRLLARLENLNNEIHEIE |
| | KMWLFIKKKEEILARGGSGDPPPPNPNDPPPPNPND |
| SEQ ID NO. 34 | GSWQTWNAKWDQWSNDWNAWRSDWQAWKDDWARLRALLMGGRLLARLENLN |
| | NEIHEIEKMWLFIKKKEEILARGGSGDPPPPNPNDPPPPNPND |
| SEQ ID NO. 35 | GSWDNWNNNWDNWYNNWDNWNNNWDNWNNNWDNLRALLMGGRLLARLENL |
| | NNEIHEIEKMWLFIKKKEEILARGGSGDPPPPNPNDPPPPNPND |
| SEQ ID NO. 36 | GSWNHWDNRWNHWDNRWNHWDNRWNHWDNRWNHLRALLMGGRLLARLENL |
| | NNEIHEIEKMWLFIKKKEEILARGGSGDPPPPNPNDPPPPNPND |
| SEQ ID NO. 37 | GSDEMLRELQETNAALQDVRELLRQQVRQITFLRALLMGGRLLARLNNIDDHINNID |
| | DYINNIDDHINNIDDHINNIDDHINNIDDHINNIDDHINNVARGGSGDPPPPNPNDPPPP |
| | NPND |
| SEQ ID NO. 38 | GSWQTWNAKWDQWSNDWNAWRSDWQAWKDDWARLRALLMGGRLLARLNNID |
| | DHINNIDDYINNIDDHINNIDDHINNIDDHINNIDDHINNIDDHINNVARGGSGDPPPPN |
| | PNDPPPPNPND |
| SEQ ID NO. 39 | GSWDNWNNNWDNWYNNWDNWNNNWDNWNNNWDNLRALLMGGRLLARLNNI |
| | DDHINNIDDYINNIDDHINNIDDHINNIDDHINNIDDHINNIDDHINNVARGGSGDPPPP |
| | NPNDPPPPNPND |
| SEQ ID NO. 40 | GSWNHWDNRWNHWDNRWNHWDNRWNHWDNRWNHLRALLMGGRLLARLNNID |
| | DHINNIDDYINNIDDHINNIDDHINNIDDHINNIDDHINNIDDHINNVARGGSGDPPPPN |
| | PNDPPPPNPND |
| SEQ ID NO. 41 | GSDEMLRELQETNAALQDVRELLRQQVRQITFLRALLMGGRLLARLNNIDDHINNL |
| | NNEIHEIEKMWLFVKEEIKEVKEEIKELARGGSGDPPPPNPNDPPPPNPND |
| SEQ ID NO. 42 | GSWQTWNAKWDQWSNDWNAWRSDWQAWKDDWARLRALLMGGRLLARLNNID |
| | DHINNLNNEIHEIEKMWLFVKEEIKEVKEEIKELARGGSGDPPPPNPNDPPPPNPND |
| SEQ ID NO. 43 | GSWDNWNNNWDNWYNNWDNWNNNWDNWNNNWDNLRALLMGGRLLARLNNI |
| | DDHINNLNNEIHEIEKMWLFVKEEIKEVKEEIKELARGGSGDPPPPNPNDPPPPNPND |
| SEQ ID NO. 44 | GSWNHWDNRWNHWDNRWNHWDNRWNHWDNRWNHLRALLMGGRLLARLNNID |
| | DHINNLNNEIHEIEKMWLFVKEEIKEVKEEIKELARGGSGDPPPPNPNDPPPPNPND |
| SEQ ID NO. 45 | GSDEMLRELQETNAALQDVRELLRQQVRQITFLRCLLMGGRLLCRLEELERR |
| | LEELERRLEELERAINTVDLELAALRRRLEELARGGSGDPPPPNPNDPPPPNPND |
| SEQ ID NO. 46 | GSDEMLRELQETNAALQDVRELLRQQVRQITFLRCLLMGGRIKEEIKEVKEEIKEVK |
| | EEIKEVKEEIKEVKEEIKEEIKEVKELARGGSGDPPPPNPNDPPPPNPND |
| SEQ ID NO. 47 | GSDNWNNNWDNWYNNWDNWNNNWDNWNNNWDNWNNNWMGGRIKEEIKEVKEEIKE |
| | VKEEIKEVKEEIKEVKEE′IKEEIKEVKELARGGSGDPPPPNPNDPPPPNPND |

TABLE 7
(P. falciparum epitopes)

| SEQ ID NOs. | Sequence |
|---|---|
| SEQ ID NO 95 | GSDEMLRELQETNAALQDVRELLRQQVRQITFLRALLMGGRLLARLEELERRLEELE |
| | RRLEELERAINTVDLELAALRRRLEELARGGSGNANPNANPNANP |
| SEQ ID NO. 96 | GSWQTWNAKWDQWSNDWNAWRSDWQAWKDDWARLRALLMGGRLLARLEELE |
| | RRLEELERRLEELERAINTVDLELAALRRRLEELARGGSGNANPNANPNANP |
| SEQ ID NO. 97 | GSWDNWNNNWDNWYNNWDNWNNNWDNWNNNWDNLRALLMGGRLLARLEEL |
| | ERRLEELERRLEELERAINTVDLELAALRRRLEELARGGSGNPNNANPNANPNANP |
| SEQ ID NO. 98 | GSWNHWDNRWNHWDNRWNHWDNRWNHWDNRWNHLRALLMGGRLLARLEELE |
| | RRLEELERRLEELERAINTVDLELAALRRRLEELARGGSGNANPNANPNANP |
| SEQ ID NO. 99 | GSDEMLRELQETNAALQDVRELLRQQVRQITFLRALLMGGRLLARLKEVKEEIKEV |
| | KEEIKEVKEEIKEVKEEIKEEIKEVKELARGGSGNANPNANPNANP |
| SEQ ID NO. 100 | GSWQTWNAKWDQWSNDWNAWRSDWQAWKDDWARLRALLMGGRLLARLKEVK |
| | EEIKEVKEEIKEVKEEIKEVKEEIKEEIKEVKELARGGSGNANPNANPNANP |
| SEQ ID NO. 101 | GSWDNWNNNWDNWYNNWDNWNNNWDNWNNNWDNLRALLMGGRLLARLKEV |
| | KEEIKEVKEEIKEVKEEIKEVKEEIKEEIKEVKELARGGSGNANPNANPNANP |
| SEQ ID NO. 102 | GSWNHWDNRWNHWDNRWNHWDNRWNHWDNRWNHLRALLMGGRLLARLKEV |
| | KEEIKEVKEEIKEVKEEIKEVKEEIKEEIKEVKELARGGSGNANPNANPNANP |
| SEQ ID NO. 103 | GSDEMLRELQETNAALQDVRELLRQQVRQITFLRALLMGGRLLARLENLNNEIHEIE |
| | KMWLFIKKKEEILARGGSGNANPNANPNANP |
| SEQ ID NO. 104 | GSWQTWNAKWDQWSNDWNAWRSDWQAWKDDWARLRALLMGGRLLARLENLN |
| | NEIHEIEKMWLFIKKKEEILARGGSGNANPNANPNANP |
| SEQ ID NO. 105 | GSWDNWNNNWDNWYNNWDNWNNNWDNWNNNWDNLRALLMGGRLLARLENL |
| | NNEIHEIEKMWLFIKKKEEILARGGSGNANPNANPNANP |
| SEQ ID NO. 106 | GSWNHWDNRWNHWDNRWNHWDNRWNHWDNRWNHLRALLMGGRLLARLENL |
| | NNEIHEIEKMWLFIKKKEEILARGGSGNANPNANPNANP |
| SEQ ID NO. 107 | GSDEMLRELQETNAALQDVRELLRQQVRQITFLRALLMGGRLLARLNNIDDHINNID |
| | DYINNIDDHINNIDDHINNIDDHINNIDDHINNIDDHINNVARGGSGNANPNANPNAN |
| | P |
| SEQ ID NO. 108 | GSWQTWNAKWDQWSNDWNAWRSDWQAWKDDWARLRALLMGGRLLARLNNID |
| | DHINNIDDYINNIDDHINNIDDHINNIDDHINNIDDHINNIDDHINNVARGGSGNANPN |
| | ANPNANP |
| SEQ ID NO. 109 | GSWDNWNNNWDNWYNNWDNWNNNWDNWNNNWDNLRALLMGGRLLARLNNI |
| | DDHINNIDDYINNIDDHINNIDDHINNIDDHINNIDDHINNIDDHINNVARGGSGNANP |
| | NANPNANP |
| SEQ ID NO. 110 | GSWNHWDNRWNHWDNRWNHWDNRWNHWDNRWNHLRALLMGGRLLARLNNID |
| | DHINNIDDYINNIDDHINNIDDHINNIDDHINNIDDHINNIDDHINNVARGGSGNANPN |
| | ANPNANP |
| SEQ ID NO. 111 | GSDEMLRELQETNAALQDVRELLRQQVRQITFLRALLMGGRLLARLNNIDDHINNL |
| | NNEIHEIEKMWLFVKEEIKEVKEEIKELARGGSGNANPNANPNANP |
| SEQ ID NO. 112 | GSWQTWNAKWDQWSNDWNAWRSDWQAWKDDWARLRALLMGGRLLARLNNID |
| | DHINNLNNEIHEIEKMWLFVKEEIKEVKEEIKELARGGSGNANPNANPNANP |
| SEQ ID NO. 113 | GSWDNWNNNWDNWYNNWDNWNNNWDNWNNNWDNLRALLMGGRLLARLNNI |
| | DDHINNLNNEIHEIEKMWLFVKEEIKEVKEEIKELARGGSGNANPNANPNANP |
| SEQ ID NO. 114 | GSWNHWDNRWNHWDNRWNHWDNRWNHWDNRWNHLRALLMGGRLLARLNNID |
| | DHINNLNNEIHEIEKMWLFVKEEIKEVKEEIKELARGGSGNANPNANPNANP |
| SEQ ID NO. 115 | GSDEMLRELQETNAALQDVRELLRQQVRQITFLRCLLMGGRLLCRLEELERR |
| | LEELERRLEELERAINTVDLELAALRRRLEELARGGSGNANPNANPNANP |
| SEQ ID NO. 116 | GSDEMLRELQETNAALQDVRELLRQQVRQITFLRCLLMGGRIKEEIKEVKEEIKEVK |
| | EEIKEVKEEIKEVKEEIKEEIKEVKELARGGSGNANPNANPNANP |
| SEQ ID NO. 117 | GSDNWNNNWDNWYNNWDNWNNNWDNWNNNWDGGRIKEEIKEVKEEIKE |
| | VKEEIKEVKEEIKEVKEE′IKEEIKEVKELARGGSGNANPNANPNANP |
| SEQ ID NO. 118 | GSDEMLRELQETNAALQDVRELLRQQVRQITFLRALLMGGRLLARLEELERRLEELE |
| | RRLEELERAINTVDLELAALRRRLEELARGGSGNANPNANPNANPNANP |
| SEQ ID NO. 119 | GSWQTWNAKWDQWSNDWNAWRSDWQAWKDDWARLRALLMGGRLLARLEELE |
| | RRLEELERRLEELERAINTVDLELAALRRRLEELARGGSGNANPNANPNANPNANP |
| SEQ ID NO. 120 | GSWDNWNNNWDNWYNNWDNWNNNWDNWNNNWDNLRALLMGGRLLARLEEL |
| | ERRLEELERRLEELERAINTVDLELAALRRRLEELARGGSGNANPNANPNANPNANP |
| SEQ ID NO. 121 | GSWNHWDNRWNHWDNRWNHWDNRWNHWDNRWNHLRALLMGGRLLARLEELE |
| | RRLEELERRLEELERAINTVDLELAALRRRLEELARGGSGNANPNANPNANPNANP |
| SEQ ID NO. 122 | GSDEMLRELQETNAALQDVRELLRQQVRQITFLRALLMGGRLLARLKEVKEEIKEV |
| | KEEIKEVKEEIKEVKEEIKEEIKEVKELARGGSGNANPNANPNANPNANP |
| SEQ ID NO. 123 | GSWQTWNAKWDQWSNDWNAWRSDWQAWKDDWARLRALLMGGRLLARLKEVK |
| | EEIKEVKEEIKEVKEEIKEVKEEIKEEIKEVKELARGGSGNANPNANPNANPNANP |
| SEQ ID NO. 124 | GSWDNWNNNWDNWYNNWDNWNNNWDNWNNNWDNLRALLMGGRLLARLKEV |
| | KEEIKEVKEEIKEVKEEIKEVKEEIKEEIKEVKELARGGSGNANPNANPNANPNANP |
| SEQ ID NO. 125 | GSWNHWDNRWNHWDNRWNHWDNRWNHWDNRWNHLRALLMGGRLLARLKEV |
| | KEEIKEVKEEIKEVKEEIKEVKEEIKEEIKEVKELARGGSGNANPNANPNANPNANP |
| SEQ ID NO. 126 | GSDEMLRELQETNAALQDVRELLRQQVRQITFLRALLMGGRLLARLENLNNEIHEIE |
| | KMWLFIKKKEEILARGGSGNANPNANPNANPNANP |
| SEQ ID NO. 127 | GSWQTWNAKWDQWSNDWNAWRSDWQAWKDDWARLRALLMGGRLLARLENLN |
| | NEIHEIEKMWLFIKKKEEILARGGSGNANPNANPNANPNANP |
| SEQ ID NO. 128 | GSWDNWNNNWDNWYNNWDNWNNNWDNWNNNWDNLRALLMGGRLLARLENL |
| | NNEIHEIEKMWLFIKKKEEILARGGSGNANPNANPNANPNANP |
| SEQ ID NO. 129 | GSWNHWDNRWNHWDNRWNHWDNRWNHWDNRWNHLRALLMGGRLLARLENL |
| | NNEIHEIEKMWLFIKKKEEILARGGSGNANPNANPNANPNANP |
| SEQ ID NO. 130 | GSDEMLRELQETNAALQDVRELLRQQVRQITFLRALLMGGRLLARLNNIDDHINNID |
| | DYINNIDDHINNIDDHINNIDDHINNIDDHINNIDDHINNVARGGSGNANPNANPNAN |
| | PNANP |
| SEQ ID NO. 131 | GSWQTWNAKWDQWSNDWNAWRSDWQAWKDDWARLRALLMGGRLLARLNNID |
| | DHINNIDDYINNIDDHINNIDDHINNIDDHINNIDDHINNIDDHINNVARGGSGNANPN |
| | ANPNANPNANP |
| SEQ ID NO. 132 | GSWDNWNNNWDNWYNNWDNWNNNWDNWNNNWDNLRALLMGGRLLARLNNI |
| | DDHINNIDDYINNIDDHINNIDDHINNIDDHINNIDDHINNIDDHINNVARGGSGNANP |
| | NANPNANPNANP |
| SEQ ID NO. 133 | GSWNHWDNRWNHWDNRWNHWDNRWNHWDNRWNHLRALLMGGRLLARLNNID |
| | DHINNIDDYINNIDDHINNIDDHINNIDDHINNIDDHINNIDDHINNVARGGSGNANPN |
| | ANPNANPNANP |
| SEQ ID NO. 134 | GSDEMLRELQETNAALQDVRELLRQQVRQITFLRALLMGGRLLARLNNIDDHINNL |
| | NNEIHEIEKMWLFVKEEIKEVKEEIKELARGGSGNANPNANPNANPNANP |
| SEQ ID NO. 135 | GSWQTWNAKWDQWSNDWNAWRSDWQAWKDDWARLRALLMGGRLLARLNNID |
| | DHINNLNNEIHEIEKMWLFVKEEIKEVKEEIKELARGGSGNANPNANPNANPNANP |
| SEQ ID NO. 136 | GSWDNWNNNWDNWYNNWDNWNNNWDNWNNNWDNLRALLMGGRLLARLNNI |
| | DDHINNLNNEIHEIEKMWLFVKEEIKEVKEEIKELARGGSGNANPNANPNANPNANP |
| SEQ ID NO. 137 | GSWNHWDNRWNHWDNRWNHWDNRWNHWDNRWNHLRALLMGGRLLARLNNID |
| | DHINNLNNEIHEIEKMWLFVKEEIKEVKEEIKELARGGSGNANPNANPNANPNANP |
| SEQ ID NO. 138 | GSDEMLRELQETNAALQDVRELLRQQVRQITFLRCLLMGGRLLCRLEELERR |
| | LEELERRLEELERAINTVDLELAALRRRLEELARGGSGNANPNANPNANPNANP |
| SEQ ID NO. 139 | GSDEMLRELQETNAALQDVRELLRQQVRQITFLRCLLMGGRIKEEIKEVKEEIKEVK |
| | EEIKEVKEEIKEVKEEIKEEIKEVKELARGGSGNANPNANPNANPNANP |
| SEQ ID NO. 140 | GSDNWNNNWDNWYNNWDNWNNNWDNWNNNWDGGRIKEEIKEVKEEIKE |
| | VKEEIKEVKEEIKEVKEE′IKEEIKEVKELARGGSGNANPNANPNANPNANP |

A non-exhaustive list of additional target peptides suitable for use in the various embodiments of this invention include SEQ ID NO 48 through SEQ ID NO 94 which are part of the sequence listing of this application and are incorporated by reference in their entirety herein.

The peptide sequences that may be coupled with SAPNs of the invention include modifications of the sequences identified herein such as, for example, by conservative substitution of one or more amino acids that do not otherwise reduce (or may enhance) immunogenicity and/or assist in the formation of the coupled-SAPN structure.

Another embodiment of the invention comprises nucleic acid sequences that encode peptide sequences of the various embodiments of the invention that are to be coupled with SAPNs. Nucleic acid sequences include vectors and other mechanisms that assist in cloning and/or replication (recognizing the concept of codon wobble) of the sequence, including purified and synthetic sequences that may be derived or predicted from other sequences.

The optimal density of B cell epitopes on SAPN for producing the most potent antibody response in the absence of adjuvant. The density of repetitively displayed antigens has long been known to be a critical determinant of initial B cell responses and studies have confirmed this. To determine the optimal density for the type of epitope display particular to SAPN, two types of SAPN modifications are described that influence the epitope density: 1) variation of the length of the core self-assembly domain, which effectively varies the tangential density of the displayed epitopes, and 2) variation of the ratio of epitope-containing and epitope-empty peptides.

Inclusion of specific CD4⁺ and CD8⁺ T cell epitopes on the prototype SAPN will improve the potency of the vaccine. Both the N-terminus and C-terminus of the self-assembly polypeptide are exposed on the SAPN surface. In one embodiment of the invention the SAPN contains the immunodominant P. berghei CSP B cell epitope, (DPPPPNPN)₂D (SEQ ID NO 7), fused to the carboxyl terminus of the self-assembly polypeptide. Known and well-studied P. berghei CSP T cell epitopes (as discussed herein by way of non-limiting example) are engineered and fused to the amino terminus of the NCS (designed for optimal promiscuity, processing and presentation).

Modification of the prototype SAPN malaria vaccine that was protective in the mouse model to make it suitable for administration to humans. The SAPN building block polypeptide is comprised of a pentameric coiled-coil domain and a trimeric coiled-coil domain separated by a linker; multimerization via the coiled-coil sequences results in the assembly of an icosahedral particle consisting of 60 polypeptides. The pentameric sequence of the prototype SAPN was derived from rat COMP having a human ortholog. This pentameric coiled-coil sequence is replaced with another well studied and characterized pentameric sequence, the tryptophan zipper, to make the SAPN vaccine suitable for human administration by reducing the risk for autoimmunity.

Design and production of a P. falciparum (Pf) CSP SAPN vaccine based on the known immunodominant PfCSP B-cell epitope sequence (NANP)₃ (SEQ ID NO 93), the universal PfCSP Th epitope, T*(EYLNKIQNSLSTEWSPCSVT) (SEQ ID NO 8) and the PfCSP CD4+ T cell epitope, T1 ([DPNANPNV]₂) (SEQ ID NO 9). The T* epitope binds to multiple human HLA-DR class II molecules in vitro and elicits Th cells in a broad range of murine major histocompatibility complex (MHC) backgrounds. Processes to produce and purify the vaccine that allow manufacture under cGMP conditions and well known within the industry may be adapted to incorporate he various engineering methods encompassed by the embodiments of this invention.

### Malaria SAPN Vaccine

The malaria vaccine of the invention is based on self-assembling polypeptide nanoparticles (SAPN). The biophysical nature of the assembly of SAPN is understood, and the helix ends are well-suited for displaying peptidic epitopes, providing wide latitude for designs without obvious technical limitations for displaying hundreds of T and B cell epitopes per particle. The flexible SAPN platform can be easily modified to optimize the protein part of the vaccine. As a significant improvement over the prior art, the various embodiments of the invention present a flexible nanoparticle vaccine capable of displaying multiple copies of epitopes of interest to produce a high titer, boostable and protective immune response against a lethal sporozoite challenge without adjuvant.

Thus, SAPN represent an ideal model system to carefully establish the correlation between the size of the immunogen, the density of the displayed antigens, and the relative strength of the immune response they elicit, as size can be fine tuned from 15 nm to 30 nm diameter, corresponding to the size of larger protein complexes and VLPs, respectively; in this size range, the immune response to antigens displayed in repetitive arrays might be expected to vary. Experiments have shown that SAPN containing the P. berghei CSP immunodominant B cell epitope induce a sterile protective immune response without the need for any adjuvant. This is believed to be the first example of an epitope-based malaria vaccine that elicits an immune response without an adjuvant, which can be boosted by second and third injections, and has no associated adverse events.

The nanoparticles may be compounded with a pharmaceutically acceptable carrier for administration to the patient (which is preferably a human, a primate or another mammal). Administration is preferably iv, but may be subq, ip, oral, or as may be most efficacious. Preferred pharmaceutically acceptable carriers are determined empirically, and include, water, glycerin, starch, carbohydrates, glycol, fillers, flavor crystals and other forms of flavoring, colorant, buffers, and stabilizers. The vaccine may also be packaged for extended release when administered such as in capsules or multiple microcapsules designed for the release of active agent for a set period of time and under a set period of conditions.

The single chain polypeptide building block of SAPN self-assemble and are highly immunogenic. The great advantage of SAPN over VLP include a potential lower cost of manufacturing and greater flexibility for the display of a variety of epitope types in a variety of configurations for optimizing immunogenicity. Fusing a single B cell epitope to the C-terminus provides for the display of 60 epitope copies on the particle surface. Strings of B cell epitopes in tandem can be fused to both the N- and C-termini providing for the display of up to hundreds of B cell epitopes on the surface of a single particle. Similarly, a large number and variety of class I and class II T cell epitopes can be fused to the termini of the linear polypeptide or engineered into the nanoparticle core. SAPN can also be assembled from different linear peptides, as another means of providing epitope diversity, albeit at the cost of additional manufacturing trains and cost. Small proteins or domains can also be displayed on the surface of SAPN; for example, to display polypeptide TLR ligands for activating dendritic cells.

### Flexible Design of Core Nanoparticles

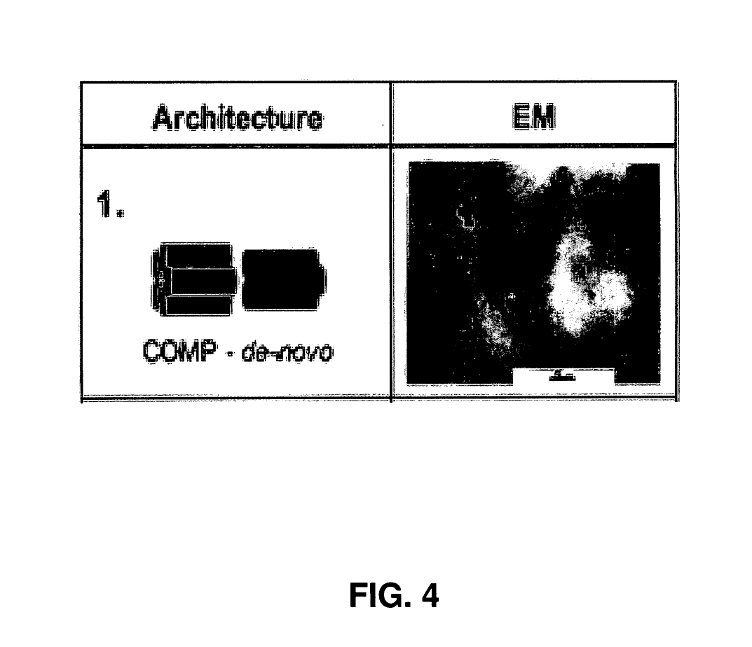

About 50 different SAPN have been rationally designed, cloned, expressed and purified (see [40, 44, 45]). These studies have demonstrated the high degree of tolerance of nanoparticles for accommodating different sizes and types of oligomerization domains. The class of nanoparticle architecture that has been studied with respect to nanoparticle application to malarial vaccines is illustrated in FIG. 4 next to electron micrographic images of the nanoparticle. This class comprises a single pentamerization domain paired with a de novo sequence self-assembled into nanoparticles of the expected size (FIG. 4).

The majority of the SAPN produced (38 sequences—including the malaria SAPN with the P. berghei epitope) belong to architecture 1 (FIG. 4). The self-assembly core of this architecture is comprised of a pentameric oligomerization domain (COMP sequence from rattus norvegicus [46]) that was slightly modified (36 N-terminal aa), a linker sequence, and a trimeric coiled-coil sequence (26 C-terminal aa), all designed de novo [42, 43].

Molecular design considerations indicated that fixing the angle between the pentameric and trimeric sequences on the linear polypeptide building block might be beneficial for nanoparticle assembly. To do this, computer graphics and modeling programs were used to design a disulfide bridge between the pentameric and trimeric sequences. Cysteines were placed at positions (f) in the heptad repeats of the respective coiled coils and one turn away from the respective helix ends, aiming to form a disulfide bridge without disturbing the coiled-coil geometry of the two oligomerization domains (compare also [47]). The “fixed-angle” design was included in the prototype SAPN vaccine used for proof-of-concept efficacy studies.

To prepare the prototype SAPN vaccine with the “fixed-angle” helices, the refolding procedure was optimized and succeeded in obtaining homogeneous SAPN preparations. The molecular weight and size of these particles, determined by analytical ultracentrifugation and dynamic light scattering, respectively, are in agreement with nanoparticles having an icosahedral symmetry composed of 60 peptide chains. Judged by EM, the SAPN form nanoparticles of roughly homogeneous size and spherical appearance, with a diameter of about 16 nm, in good agreement with the value predicted from computer modeling. Thermal denaturation experiments, using CD-spectroscopy, demonstrate that these SAPN are extremely thermostable. The α-helical signal at 90° C. is only 20% lower than at room temperature and nearly 100% of the initial signal is recovered up on cooling.

A problematic characteristic of constructs having the “fixed-angle” helices was that a large fraction of the SAPN started to aggregate after a few days under oxidizing conditions as determined using particle size analysis (Dynamic Light Scattering). However, most of them remained soluble under reducing conditions. This behavior was particularly pronounced constructs that contained an additional disulfide bridge in the epitope. A major reason for the aggregation was disulfide cross-linking between SAPN. Therefore, the two cysteines in the nanoparticle core were replaced with alanine residues. These “cysteine-free” linear polypeptide building blocks self-assemble into homogeneous nanoparticles that remain non-aggregated in solution over a period of several months (the monitoring for potential aggregation by EM and DLS will continue for several more months) in the absence of a reducing agent. Accordingly, this design was used as the basis for all of the SAPN.

To reduce the risk for aggregation, epitopes are selected based on low hydrophobicity and the absence of runs of charged residues. Second, excipients for preventing aggregation that are common constituents of vaccines are tested. Glycerol significantly improved the solubility of certain SAPN. Finally, the high degree of thermal stability of SAPN indicates that they likely can be further stabilized using glassification technology (Cambridge Biostability Limited). The design architecture of the SAPN shows considerable flexibility, and allows interchangeability of oligomerization domains. The biophysical stability of the SAPN is so high that they resist denaturation at 90° C. Optimization of design can effectively address SAPN solubility issues. SAPN therefore, are a Platform for Malaria Vaccines.

The following examples illustrate embodiments of the invention, but should not be viewed as limiting the scope of the invention.

### EXAMPLES

### Example 1

In this example we designed and produced a SAPN comprising the core self-assembly domain sequence (a pentameric and trimeric coiled-coil oligomerization domain joined by a short linker) that displays on its surface two repeats of the P. berghei malaria B cell epitope (DPPPPNPN)₂D (SEQ ID NO 7). Mice were immunized with these particles with and without adjuvant, which showed that nanoparticles are highly immunogenic and induce protective immunity to lethal challenge with infective sporozoites in the absence of adjuvant. Long lasting antibodies were produced as demonstrated by protection of mice up to at least 3 months after a third injection. Furthermore, B-cell maturation occurs by the induction of Ig isotype switch from IgM to IgG2a/2b.

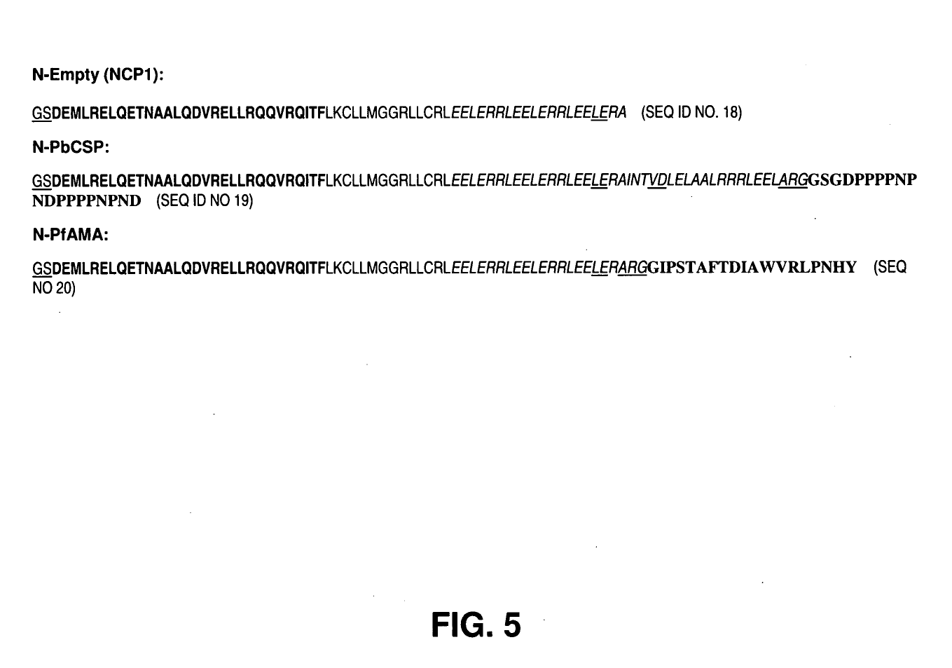

To establish the utility of the design, the three linear, self-assembling polypeptides were produced as depicted in FIG. 5. N-Empty (NCP1) contains only the core assembly domain. N-PbCSP displays the di-repeat of the P. berghei CSP, which is known to be a protective antigen on the core self-assembly domain. N-PfAMA is a negative control epitope from P. falciparum, which in not protective for P. berghei, displayed on the core self-assembly domain. The epitopes are shown in bold serif font. The underscored amino acids are encoded by DNA sequences that are restriction sites to facilitate cloning. The sequences of the pentameric coiled-coil are shown in bold sans-serif font. Protein sequences encoding the linker segment are in plain font. Protein sequences encoding the trimeric coiled-coil are in italicized font.

A number of studies have demonstrated that the P. berghei CSP repeat, when administered with adjuvant, can induce protective antibodies in immunized mice. As a negative control, the P. falciparum AMA-1 mimotope sequence [48] was used for the P. berghei experiments and does not exist on the P. berghei AMA-1 protein.

The “potency assay” is based on: 1) protective efficacy to lethal challenge of mice immunized with live P. berghei sporozoite-stage parasites, and 2) antibody titer after one, two and three doses of vaccine with particular emphasis on either sero-conversion after one dose of vaccine or end-point titer and protection from challenge after 3 doses of vaccine, at 2 week intervals.

Protective Efficacy of SAPN in the P. berghei Mouse Malaria Model.

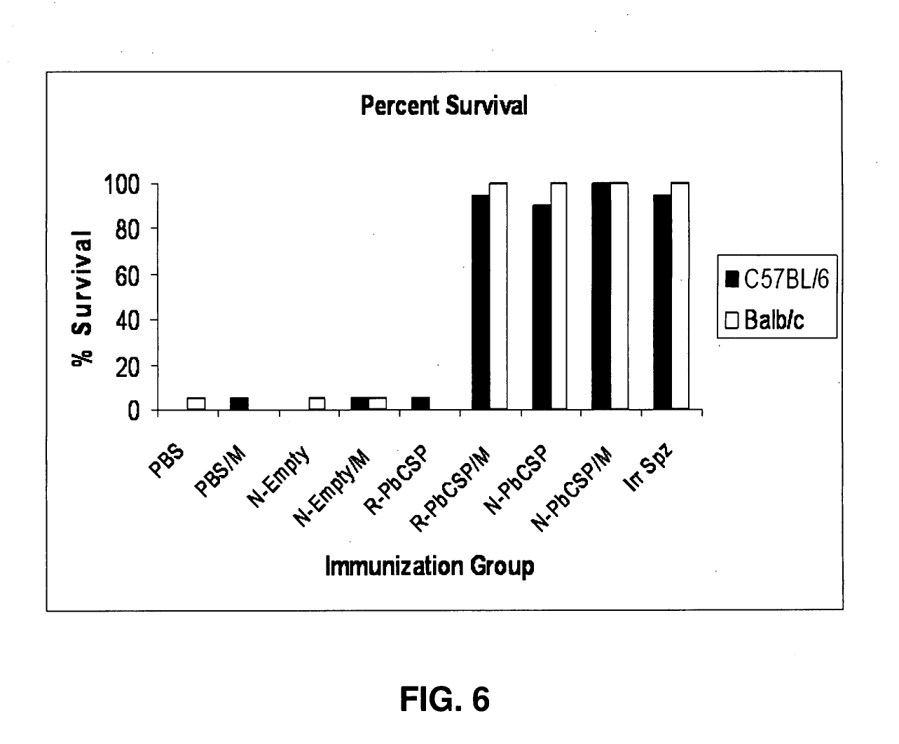

The protective efficacy of a SAPN displaying the P. berghei CSP B cell epitope peptide (N-PbCSP) was compared with that of full length, recombinant, PbCSP protein (R-PbCSP) in two strains of mice with different MHC backgrounds. SAPN, or recombinant protein in PBS, or the adjuvant Montanide ISA 720, were injected intramuscularly (i.m.). The negative control was a nanoparticle core without a fused epitope (N-Empty). The positive control for a protective immunization was irradiated sporozoites. C57BL/6 or Balb/c mice receiving N-PbCSP, with or without adjuvant were completely protected from a lethal challenge with P. berghei sporozoites (FIG. 6). Only immunization with irradiated sporozoites or recombinant PbCSP protein in Montanide ISA 720™ imparted an equivalent level of protection. Recombinant protein without adjuvant, N-empty, or adjuvant alone induced no protective responses. A surprising finding was the sterile protection provided by N-PbCSP immunizations without adjuvant. This was not expected, and in fact this group was included in the experimental design as a control for the immunization with the N-PbCSP with Montanide ISA-720™. Comparable results were obtained in three different experiments.

Parasitemic Mice (Balb/c) Vaccinated with SAPN with or without Adjuvant are Provided Sterile Protection.

The P. berghei CSP protein is only expressed on the initial stage of the parasite infection. Once the sporozoite invades the liver, hepatocyte merozoites are formed that do not have CSP, and therefore any blood stage parasites that are seen after the hepatocyte incubation phase (3-4 days) will multiply without immune hindrance. To determine that all mice that were protected did not develop parasites, and then self-cure (a rare event with this parasite infection) blood stage parasitemia was followed.

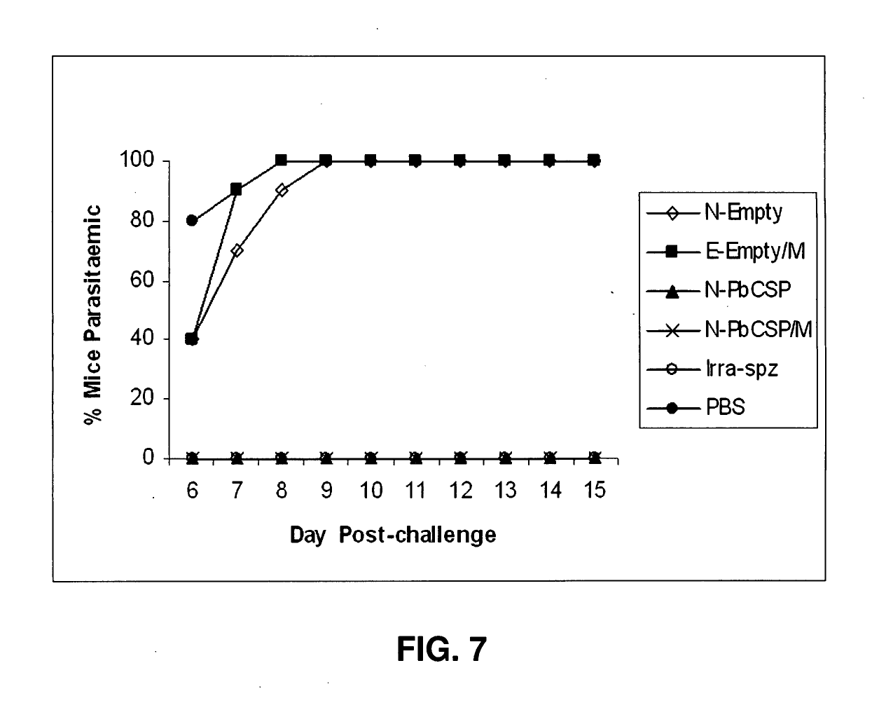

Mice were immunized with N-Empty or N-PbCSP in either PBS or Montanide ISA 720™. As a control for protection, mice were immunized with irradiated sporozoites. All mice were challenged with 1,000 live sporozoites. Greater then 95% of mice receiving N-PbCSP in PBS or N-PbCSP in Montanide ISA 720™ were sterilely protected (defined as not developing parasitemia by microscopic detection of parasites on stained thin film blood smears) in response to challenge with infective sporozoites (FIG. 7). This level of protection is representative of the protection seen with irradiated sporozoite vaccination.

Antibody Titers Induced by SAPN Correlate with Protection.

The SAPN for this experiment contains only a B cell epitope, and therefore antibodies are expected to be produced against that epitope. The blood antibody level for correlation with the protective response. Published results using VLP containing the same P. berghei CSP epitope peptides was unable to induce high titer antibody without CFA adjuvant even after several immunizations.

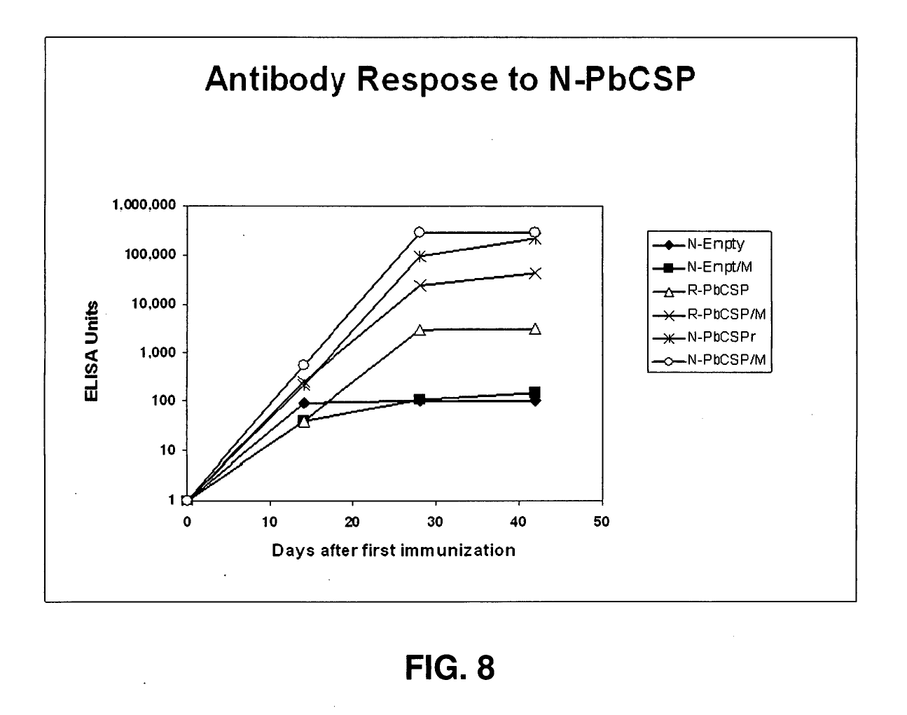

Mice (C57BL/6 or Balb/c) were immunized with 10 μg of antigen with or without adjuvant. Titers of total IgG in serum two weeks after each immunization were measured by ELISA using synthetic PbCSP repeat peptide as the plate antigen (FIG. 8). In all cases low levels of antibody were detected two weeks after the initial injection. Two weeks after the second immunization, antibody titers to N-PbCSP in Montanide ISA 720™ reached a maximum level (300,000 ELISA Units) that was not boostable. The N-PbCSP nanoparticle delivered without adjuvant was 100,000 ELISA Units and boostable to 250,000 ELISA Units after a third immunization. IgG specific response to R-PbCSP in Montanide ISA 720™ after 3 doses reached a titer 25% of that seen with a two injection of SAPN-PbCSP without adjuvant and a 5 to 6 times lower than that achieved by three doses of the N-SAPN-PbCSP with or without adjuvant. It was concluded that: (1) a SAPN that repetitively displays epitope peptide induces an antigen-specific antibody response; (2) this response is of high titer after 2 doses; (3) a third dose does allow boosting of antibody titer; (4) the use of adjuvant may increase the rate of immune response to achieve maximum values after two doses but there is no significant difference after 3 doses (p<0.05); and (5) comparison of antibody titers achieved in this set of experiments and protection data achieved in FIG. 6 show that protective immunity is, in this model system, highly correlated to antibody level against the PbCSP B cell epitope.

### Passive Transfer of Serum and Splenocytes.

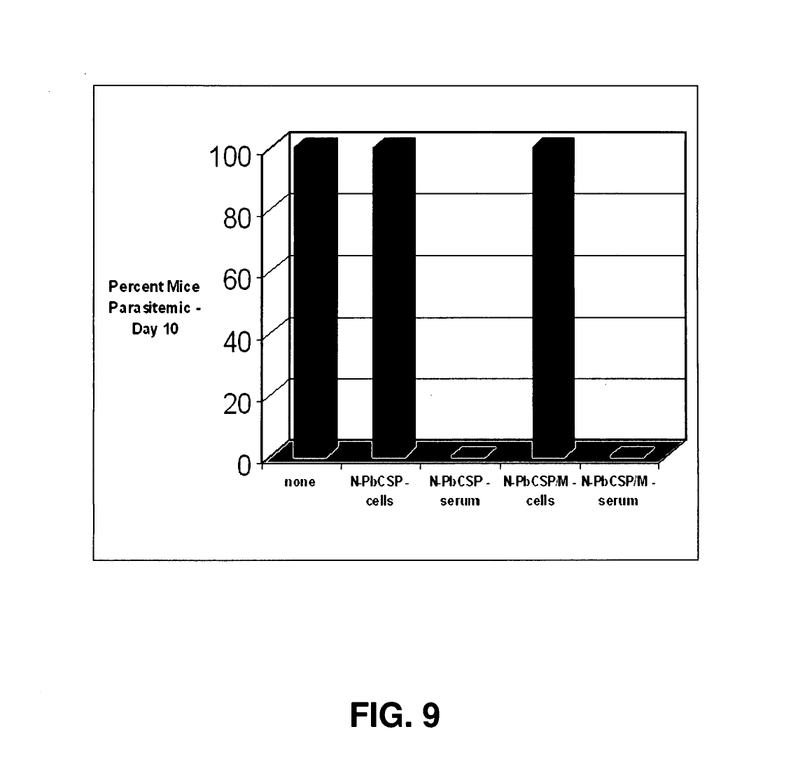

Protection was highly correlated with antibody level so we predicted that serum, but not cells, should transfer immunity. Ten Balb/c or C57BL/6 mice were immunized with N-PbCSP in the presence or absence of Montanide ISA-720. One week after the third immunization, animals were sacrificed and blood drawn from the heart for processing of serum and splenocytes harvested from the spleen. Serum or splenocytes from the ten animals were pooled. Groups of ten naïve Balb/c or C57BL/6 mice were immunized with 100 μl pooled serum (non-diluted), 10 million cells or both. The infectivity control group received neither cells nor serum. Animals were challenged with live sporozoites after the transfer. Only mice receiving serum were protected from sporozoite challenge (FIG. 9). This was expected because only a B cell epitope was used in the vaccine construct. It was important however to do this experiment because it is known that CSP specific T cells themselves can impart immunity to sporozoite challenge in Balb/c mice. Thus this model platform allows evaluation of new nanoparticles that contain T cell epitopes.

Duration of Protective Efficacy After Immunization. Groups of Balb/c and C57BL/6 mice (n=5) were immunized with 3 doses (10 μg/dose) of N-PbCSP and challenged at 1 month and then again at 3 months after the final immunization dose. In both groups 100% of the mice survived challenge. Two mice of each haplotype were not vaccinated and were challenged at each time point; all these mice developed parasites and died within 15 days. Immunization with N-PbCSP induces an immune response that can protect mice from lethal challenge for a period of at least 3 months after three doses. Because these mice were challenged at 1 month after the third injection, and therefore could have gotten a boost to their immune response. Additional experiments may investigate protection 3, 6 and 9 months after a third dose.

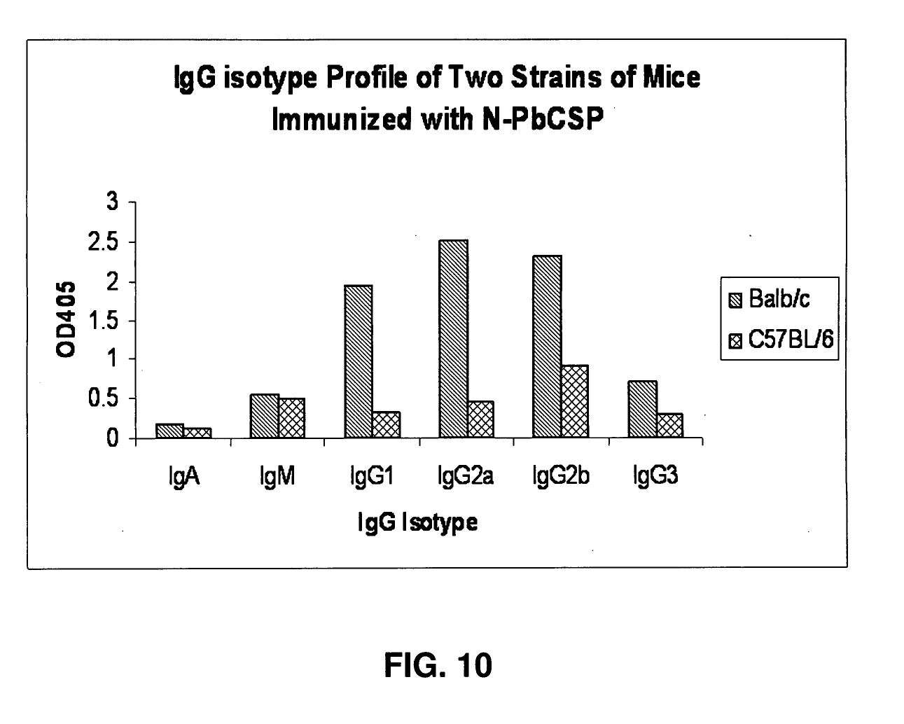

Immunoglobulin Subclass Isotype. Different antigenic stimuli tend to induce secretion of different IgG isotypes, and different isotypes have functional and structural features that make them particularly well suited to defend against specific types of pathogens. Therefore, IgG isotype determination can provide a first approximation of the effectiveness of an antigen to induce a protective antibody response. Soluble antigens tend to induce IgG1, while particulate antigens and viruses tend to induce IgG2a and IgG2b, the immunoglobulins that fix complement and are important for destruction of intracellular pathogens. The role of the complement pathway in the destruction of infected liver hepatocytes is currently unclear; however, destruction of liver heptocytes is believed to be the major mechanism of protection provided by vaccination with irradiated sporozoites. It is possible that antibodies bound to CSP proteins on the surface of infected heptocytes could fix compliment on the hepatocyte and aid in its destruction. Therefore, the isotype of IgG induced by the nanoparticles was determined. Groups (n=5) of Balb/c and C57BL/6 mice were immunized with of N-PbCSP. Serum was collected two weeks after the last dose. IgG isotype was determined by ELISA with PbCSP peptide on the plate. The nanoparticles with PbCSP B cell epitopes induced, in Balb/c mice, mostly IgG1, IgG2a and IgG2b (FIG. 10) which indicate that the functional protective effect of the vaccination may be taking place against both the sporozoite (by IgG1) and the intracellular hepatocyte stages (by IgG2a) of the parasite. The isotype maturation was not as defined in C57BL/6 mice.

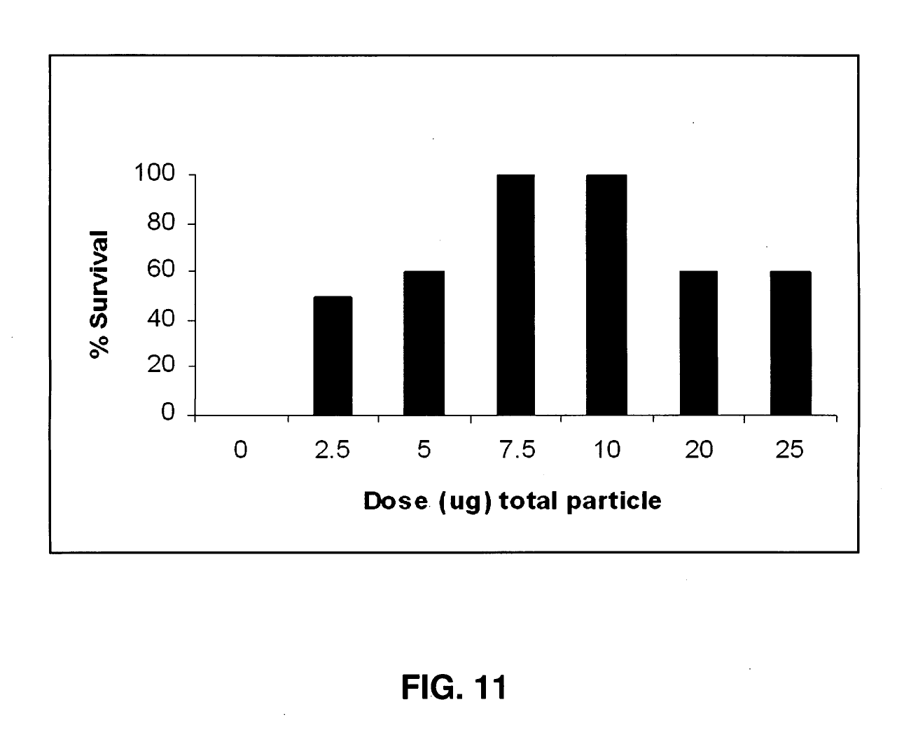

Potency of SAPN. Potency is defined here as the minimum amount of SAPN needed to induce >50% protection from challenge with 1000 live sporozoites. This value is the basis for comparing different production lots of SAPN, different methods of delivery of SAPN, and different SAPN constructs. To determine the potency of N-PbCSP, groups (n=5) mice C57BL/6 received three immunizations of 100 μl PBS containing 0.00, 2.5, 5.0, 7.5, 10.0, 20 or 25 μg N-PbCSP (w/o adjuvant). Two weeks after the third immunization they were all challenged with P. berghei sporozoites (FIG. 11).

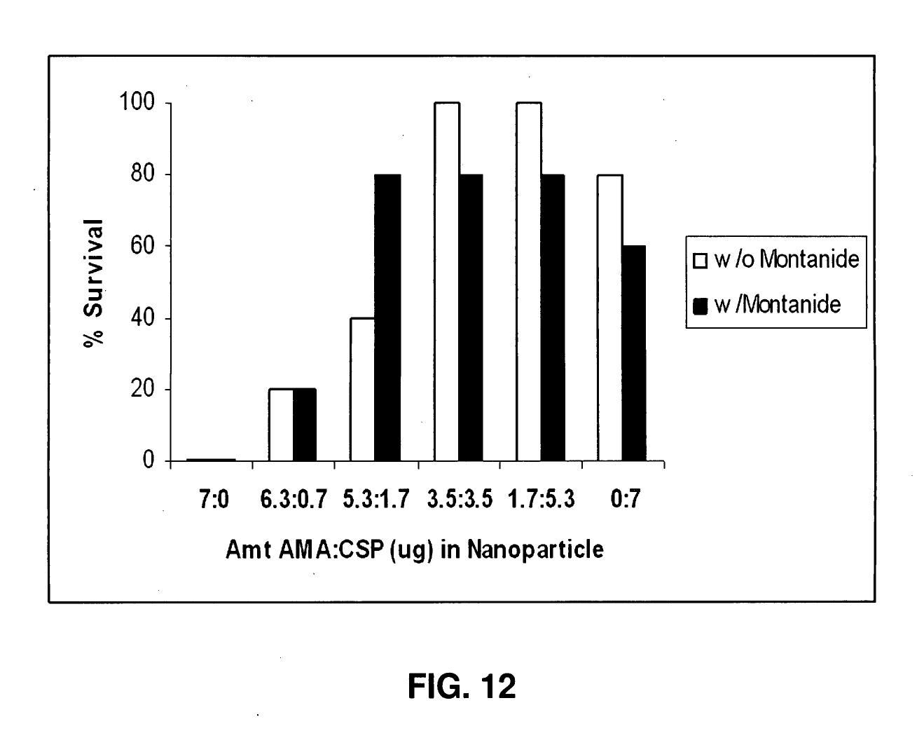

Variation of the Number of Epitopes per Particle. Divalent nanoparticles were prepared by mixing the two linear polypeptide building blocks, PfAMA and PbCSP, at different ratios under denaturing conditions, and then removing the denaturant to permit self-assembly. Six groups (n=10) C57BL/6 mice were immunized with 100 μl PBS (w/o adjuvant) containing SAPN prepared as a mixture of N-PfAMA and N-PbCSP in the following ratios (N-PfAMA/N-PbCSP): 7:0, 6.3:0.7, 5.3:1.7, 1.7:5.3, 0:7 (μg). A series of mice were also immunized with the same SAPN adjuvant with Montanide ISA 720 (FIG. 12). (1) The dose of N-PbCSP that gives >50% protection is 5.0 ug and 100% protection is 7.5 μg. (2) Increasing amounts of antigen above a certain threshold was found to be detrimental to the efficacy of the immune response. (3) The potency of the SAPN may in fact be higher if the number of target epitopes is less than 60 (the number of linear proteins per SAPN). These are only initial studies with small numbers and a single strain of mice and thus will have to be repeated with more mice, especially the Balb/c mouse strain (for studies bearing P. berghei CSP peptide T-cell epitopes which are only MHC H-2^{d} (Balb/c) specific).

### Example 2

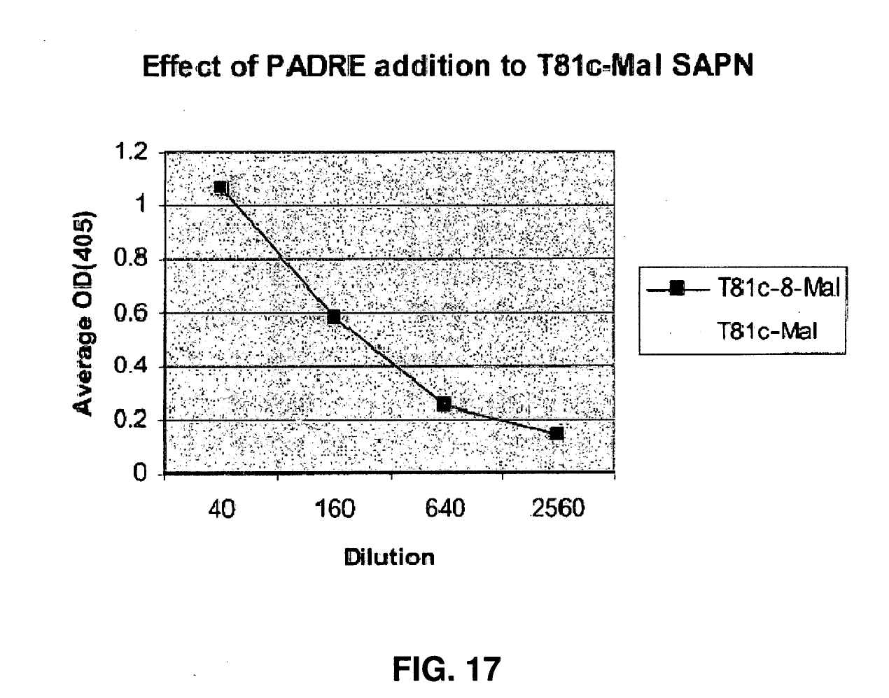

Exchanging the pentameric sequence COMP for Tryptophan Zipper (Trp zip)—effect on particle formation and immune response. The pentameric sequence COMP, though derived from mouse holds a strong similarity to the human COMP. To reduce the possibility of the induction of an autoimmune immunological reaction the COMP sequence was exchanged for a de novo designed try zip motif. These LP form excellent nanoparticles (see FIG. 4) but surprisingly were less immunogenic. As seen in FIG. 17 the nanoparticles that contained the Trp zip motif, T81c-Mal, were less immunogenic than the P4c-Mal construct that contained the COMP sequence. This immunogenicity was increased by the inclusion of the pan allelicDR epitope, PADRE, in the LP chain that makes up the SAPN T81c-8-Mal. However, in both experiments when mice were immunized with T81c-Mal or T81c-8-Mal and then challenged with 1000 live sporozoites only 60% ofthe mice survived compared to the 100% survival of mice immunized with P4c-Mal. The PADRE made only a small difference in the antibody titer. This could be because PADRE does not bind well to the H2b as it does to other MHC haplotypes. We used C57B1/6 [H2b] mice as were used by Franke, et al (Vaccine 1999 17, p1201) to show that PADRE provided T-cell help for induction of antibody to the P. yoelii CSP B-cell epitope. It should be noted however that their construct was given with the powerful adjuvant TiterMax. These constructs were delivered in PBS. The PADRE epitope has been shown to bind more strongly to H-2D (B. Livingston, J. Immunol. 2002, 168) therefore the experiments can be done with Balb/c which are H-2^{d} haplotype.

### Example 3

Determine the optimal density of B cell epitopes on SAPN for producing the most potent antibody response in the absence of an adjuvant. Antigens that cross-link surface membrane Ig are known to the efficiently activate B cells, whereas monomeric antigens tend to induce B cell tolerance in the absence of CD4+ Th cells. Studies of the epitope density of T cell-independent antigens have shown that arrays of 20-30 haptens, when optimally space by 5-10 nm, can efficiently cross-link and activate immature B cells in the absence of Th cells [33, 49, 50]. In addition, Jegerlehner et al., 2002 [51] demonstrated that IgG responses that are dependent on Th cells are also significantly dependent on epitope density. More importantly perhaps, Liu et al. have shown that antibody affinity constants are as much as 2 logs higher when antigens are displayed in optimal density arrays [39, 52]. These studies and others [35, 36, 38, 39] (FIG. 12), indicate that the density of epitopes on SAPN is an important determinant of their immunogenicity. We vary the density of B cell epitopes displayed on the SAPN surface using two strategies to identify the epitope configuration that is most immunogenic.

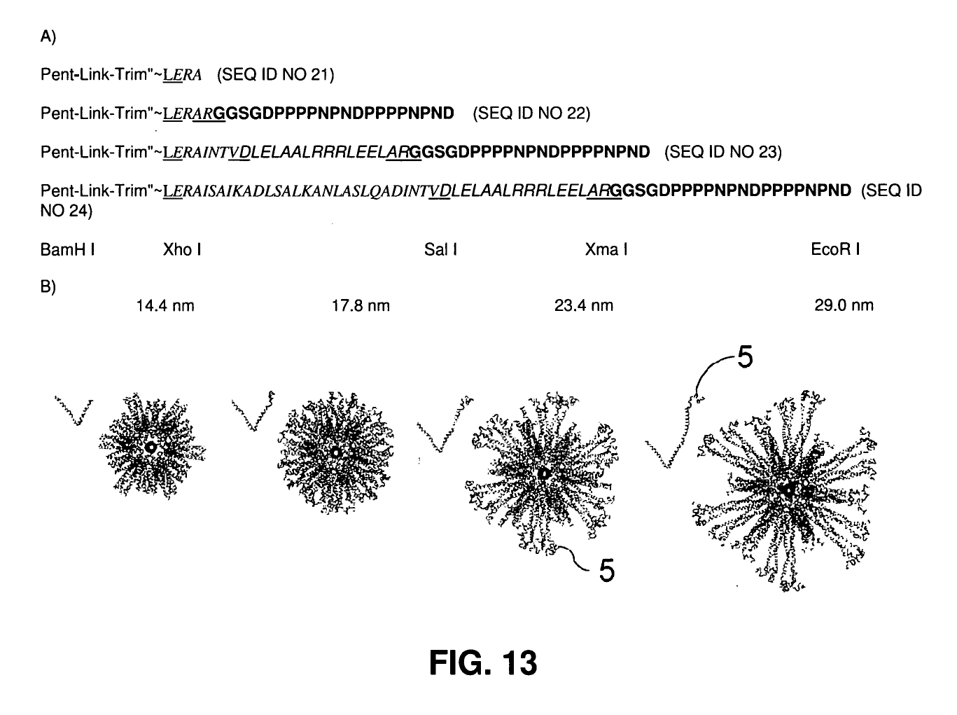

First, the radius of the nanoparticle is varied by extending the “spokes on the wheel”. The addition of extenders consisting of trimerization domains is used to increase the diameter of the SAPN, which increases the surface area without changing the number of epitopes, thus lessening the epitopes per surface area, or density. According to computer modeling, changing the length of the coiled-coil trimer leads to SAPN ranging in size from about 15 nm to 30 nm in diameter (FIG. 13).

Second, SAPN is assembled from polypeptides consisting of those lacking B cell epitopes and those containing B cell epitopes. Up to 20 different SAPN with different epitope densities on their surface are obtained by varying the ratio of these two types of polypeptides. Each SAPN meeting the specifications of homogeneity, solubility and stability is tested for potency to induce protective immunity against live sporozoite challenge in the P. berghei mouse malaria model (potency assay).

An expression cassette system was designed with different restriction sites separating each functional group to enable the addition, removal or exchange of core domains (trimer, pentamer, linker) and surface epitopes (see FIG. 13). Looking now at FIG. 13A: The initial vector contains a plasmid for the naked core SAPN composed of a pentameric coiled-coil (Pent), a linker sequence (Link) and a trimerization domain (Trim). Cutting at engineered restriction sites (underscored) with the restriction enzymes EcoR I and Xho I and subsequent insertion of the B-cell epitope of malaria (bold) generates the smallest SAPN that repetitively presents the antigen on its surface. Cutting with the restriction enzymes Xma I and Xho I and subsequent insertion of an extension of the trimeric coiled coil (serif-font, italics) generates the medium sized antigen-SAPN and further cutting with the restriction enzymes Sal I and Xho I and subsequent insertion of a second extension of the trimeric coiled coil (italics) finally generates the largest SAPN. This design allows for an easy insertion of an oligo coding for any given antigenic sequence (bold) into any of the different sized SAPN by using the Xma I and EcoR I restrictions sites. In FIG. 13B we see Computational visualizations of the different monomers and their corresponding icosahedral SAPN. The size of the SAPN increases from about 15 nm up to 30 nm and the antigenic sequence (at the C-terminus of the sequence and hence at the end of the trimeric coiled-coil helix) is displayed repetitively on the surface of the SAPN. In FIG. 13b we see the antigenic sequence 5 is engineered so that it displays along the volume circumference of the SAPN in its assembled oligmerized state.

### Method of Constructing SAPN Nanoparticles

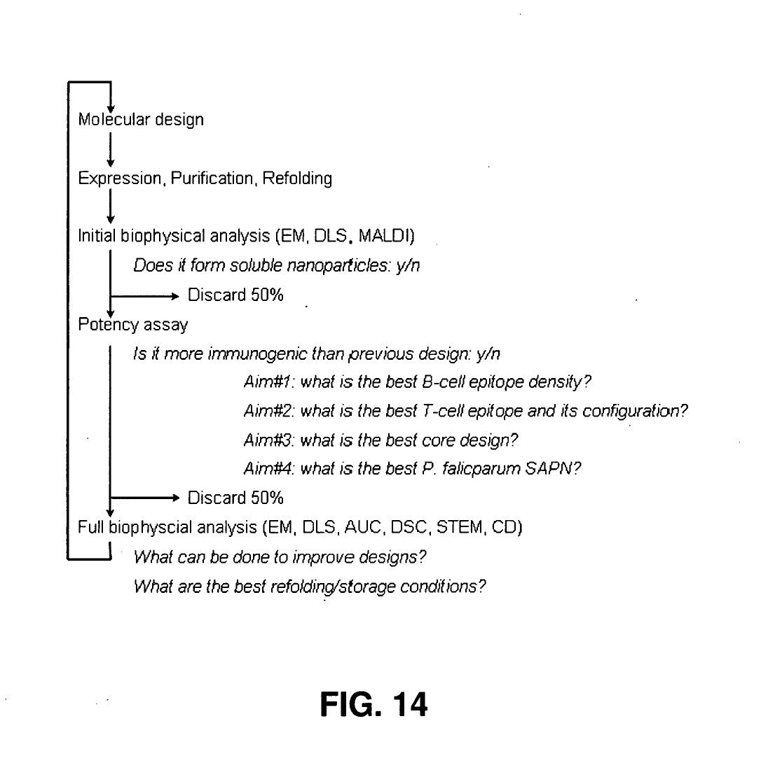

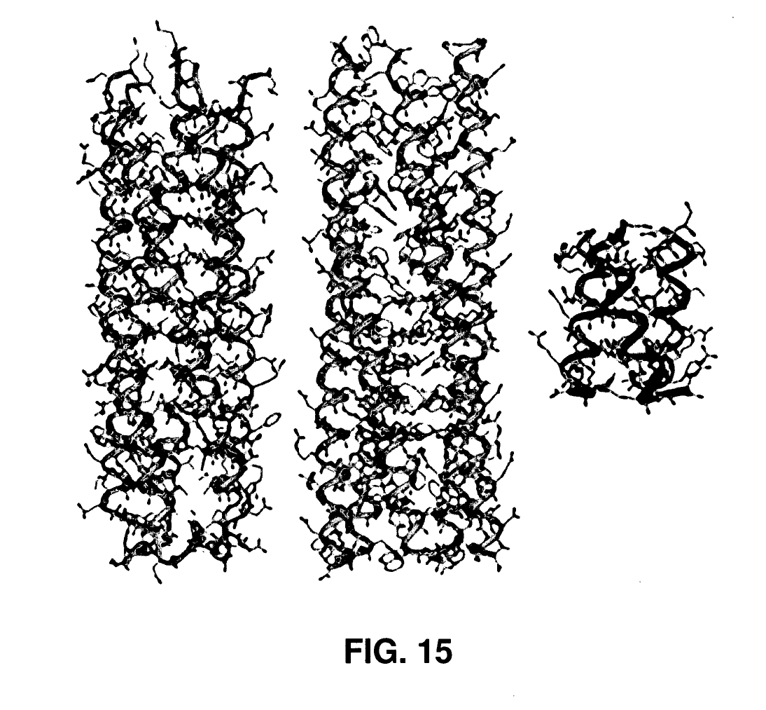

These experiments demonstrate that the SAPN platform technology allows for an improved malaria vaccine. The overall strategy follows the steps set forth on FIG. 14. First a molecular design is chosen based on the general principles of vaccine design and the engineering principles behind self-assembling peptide nanoparticles both of which have been discussed in detail supra. The oligomerization domains chosen for the design of a preferred SAPN are based on crystal structures as accessible in the protein database (see FIG. 15). The next step is incorporate the final molecular design into an expression system such as recombinant means well known in the art and also described herein The product of this expression system is then purified and placed into the appropriate stabilizing solution to maintain the desired folding orientation as determined by pH and salt concentrations. Next an initial biophysical analysis by methods known in the art is performed to determine whether the proper nanoparticle characteristics have been achieved. Subsequently, a potency assay is performed on the remaining nanoparticle solution to determine whether the particular design has optimized the following parameters: 1) optimal B-cell epitope density; 2) optimal T-cell epitope configuration; 3) optimal core design; 4) optimal overall SAPN design. Once an optimized SAPN has been determined and isolated, the SAPN is tested to determine the best refolding and storage conditions. This method may be cycled continuously with a focus on improving the design of the nanoparticles.

### Example 4

Production of SAPN with different epitope density by mixing epitope-empty and epitope-containing peptide building blocks. Six different polypeptide building blocks are made, 3 epitope-empty polypeptides, each with a different core diameter (17.8, 23.4 or 29 nm), and 3 epitope-containing polypeptides, each also with a different core diameter (17.8, 23.4 or 29 nm) that display the P. berghei di-repeat B cell epitope (DPPPPNPN)₂D (PbCSP). These two polypetides are mixed together in the presence of denaturant in the ratio of 0:100; 25:75; 50:50, 75:25 and 100:0 (PbCSP:Empty) and are allowed to self assemble (Table 3). The resulting mixed-peptide SAPN are assayed for potency in generating a protective immune response (15 SAPN in all if all pass homogeneity, solubility and stability tests). Balb/c mice are immunized 3 times with 7.5 μg of each SAPN mix (seven groups: PBS control, SAPN, and five mixed-peptide SAPN) and challenged with live sporozoites. Mice are monitored for parasitemia and antibody (Table 4).

TABLE 3
Mixed-polypeptide SAPN with different diameter cores.

| | | Peptide Building Blocks | | |
|---|---|---|---|---|
| | | (# of Trimerazation Extenders) | | |
| | SAPN (Core | A. Epitope- | B. Epitope- | |
| # | diameter) | Empty | Containing | A:B |
| 1 | 17.8 nm | 0 | 0 | 0:100; 25:75; 50:50, |
| | | | | 75:25 100:0 |
| 2 | 23.4 nm | 1 | 1 | 0:100; 25:75; 50:50, |
| | | | | 75:25 100:0 |
| 3 | 29 nm | 2 | 2 | 0:100; 25:75; 50:50, |
| | | | | 75:25 100:0 |

TABLE 4
Experimental Design of Potency Assay: P. berghei live
sporozoite challenge⁺ least quantity of SAPN that
protects =50% mice from blood stage parasitemia for 15 days

| | | VaccineDose | | | |
|---|---|---|---|---|---|
| | | (7.5 μg) i.m. | Bleed times | Chal- | |
| | | on Day 0, 14, | for immuno- | lenge^{$} | Parasitemia |
| Group | N | 28* | assays^{\hat{ }} (Days) | Day | Assay Day^{#} |
| 1 | 10 | 0 (PBS or | 0, 14, 28, 42 | 42 | 47, 49, 51, |
| | | adjuvant | | | 53, 55, 57 |
| | | alone) | | | |
| 2 | 10 | Prototype | 0, 14, 28, 42 | 42 | 47, 49, 51, |
| | | SAPN | | | 53, 55, 57 |
| 3 | 10 | 0:100 | 0, 14, 28, 42 | 42 | 47, 49, 51, |
| | | | | | 53, 55, 57 |
| 4 | 10 | 25:75 | 0, 14, 28, 42 | 42 | 47, 49, 51, |
| | | | | | 53, 55, 57 |
| 5 | 10 | 50:50 | 0, 14, 28, 42 | 42 | 47, 49, 51, |
| | | | | | 53, 55, 57 |
| 6 | 10 | 75:25 | 0, 14, 28, 42 | 42 | 47, 49, 51, |
| | | | | | 53, 55, 57 |
| 7 | 10 | 100:0 | 0, 14, 28, 42 | 42 | 47, 49, 51, |
| | | | | | 53, 55, 57 |

⁺in Balb/c
*100 microliters is injected i.m. on day 0, 14, 28; the same amount of adjuvant or buffer regardless of the antigen concentration. Amt of SAPN is adjusted if potency is outside lower or upper antigen limit.
\hat{ }ELISA unit = dilution to give OD > geometric mean + 3 SD of their respective pre-immune sera (ELISA using the synthetic peptide (DPPPNDVP)₂D (SEQ ID NO 11) as the plague antigen.
^{$}P. berghei sporozoites, received on day of challenge. Sporozoites are diluted to a final concentration of 1000 sporozoites/100 μl in PBS; Each mouse is injected i.v. with 100 μl of prepared sporozoites into the tail vein.
^{#}2 drops of blood is taken from each mouse and a thin blood film is prepared and Giemsa stained. Stained blood is examined microscopically for parasites. A mouse is considered positive for parasites if 5 blood stained parasites are detected in 200 or less 100x fields. The mouse is then sacrificed.

The potency of SAPN having various surface epitope density due to different diameter and/or ratio of epitope-containing polypeptides is determined. SAPN have been produced with a variety of different “extenders” and have produced SAPN consisting of mixtures of empty and epitope-containing linear polypeptides.

Each type of construct favors itself and therefore forms undesired ratios. Where the most potent SAPN design turns out to be a “mixed polypeptide” SAPN, the ratio of the two component polypeptides in SAPN on a particle-by-particle, population basis, using fluorescent dye-conjugated peptides and FACS is determined. However, the goal is to produce the most potent SAPN that is least expensive to manufacture, preferably, doesn't include two polypeptide manufacturing trains.

When constructs with no extender and three extenders are not made, the temperature of bacterial growth and protein induction times are varied and the assembly protocol is modified to optimize the final product.

### Example 5

Determine the extent to which the inclusion of specific CD4+ and CD8+ T cell epitopes on the prototype SAPN will improve the potency of the vaccine. SAPN can effectively present B cell epitopes of Plasmodium proteins to mice and induce high titer protective antibodies without the need for an adjuvant. The observed mature Ig response indicates that the nanoparticle core, within the rat COMP pentamerization or the trimerization domain (designed de novo), may contain a T cell epitope. However, a non-malarial T-cell epitope such as this limits the potential of the SAPN as a vaccine since these T cell epitopes would not elicit parasite-specific memory T cell anamnestic responses in naturally infected individuals, and vaccine-induced responses would not be boosted following exposure to bites from infected mosquitoes.

T cell help is sometimes needed for induction of high affinity antibodies to PbCSP B cell epitopes; T cell help is likely required for protection from sporozoite challenge; and PbCSP T cell epitopes may, independent of B cell epitopes, provide sufficient immunological stimuli to induce protection against P. berghei sporozoite challenge. Therefore, the optimum design for the incorporation of T cell epitopes into the SAPN is determined using known T cell epitope peptides from the P. berghei CSP.

Effect of the T1 epitope in various configurations on the protective efficacy of SAPN. The relative positional orientation within the nanoparticle of known CD4+ and CD8+ epitopes from P. berghei could well affect the efficiency of their presentation. A single T cell epitope, or multiple T cell epitopes, can be displayed on either the N-terminus or the C-terminus, or both, of the core self-assembly polypeptide. The T cell epitope need not be on the same polypeptide strand as the B cell epitope (cis), but is sufficient to be located on a different polypeptide (trans) in the SAPN. SAPN with a reduced density of B cell epitopes (i.e., containing “epitope-empty” or some “non-target specific epitope” linear peptides), induce a protective response. Therefore, replacement of those epitope-empty peptides with peptides that contain T cell epitopes fused to their ends do not negatively affect the potency of the SAPN. The N-PbCSP SAPN, which is exclusively comprised of linear peptides having the B cell epitope, provide complete protection at 7.5 μg/dose, but only partial protection at 2.5 and 5.0 μg/dose (see FIG. 11). With the addition of known T cell epitopes, protection at a lower dose is achieved.

For studies to optimize the placement of the CD4+ T cell (T-helper) epitope relative to the B cell epitope, the MHC H-2^{d} restricted PbCSP epitope peptide, T1, identified by Birkett et al. [53], is used. This epitope is protective on its own and can help induce high titer antibodies to the B cell epitope (DPPPPNPN)₂D (SEQ ID NO 7) in Balb/c mice. The T1 epitope sequence (KIYNRNTVNRLLAD) (SEQ ID NO 12) is added to either the amino or carboxy terminus of the prototype core nanoparticle sequence and following expression in E. coli, the purified peptide building blocks are assembled as indicated in the Table 5.

TABLE 5
Potentiation by a T epitope in different
configurations.
----- Homogeneous SAPN -----

| | SAPN Name | N-term | C-term |
|---|---|---|---|
| 1 | N-T1n | T1 | — |
| 2 | N-T1c | — | T1 |
| 3 | N-T1nBc | T1 | B |
| 4 | N-BnT1c | B | T1 |
| 5 | N-T1nBn | T1→B | — |
| 6 | N-T1cBc | — | T1→B |
| 7 | N-T1nBnT1cBc | T1→B | T1→B |

----------- Mixed SAPN -------------

| | | % T1 | % B |
|---|---|---|---|
| 8 | N-T1/PbCSP-a | 0 | 100 |
| 9 | N-T1/PbCSP-b | 25 | 75 |
| 10 | N-T1/PbCSP-c | 75 | 25 |
| 11 | N-T1/PbCSP-d | 100 | 0 |

T1 = KIYNRNTVNRLLAD(SEQ ID NO 12)
B = (DPPPPNPN)2D(SEQ ID NO7)

These experiments evaluate the ease of production and immunogenicity of SAPN with B and T cell epitopes in a variety of configurations. They additionally evaluate the importance of locating the T1 epitope on the N- or C-terminus, and whether the T cell epitope, without a B cell epitope, induces a protective immune response, as it did when the epitope was displayed in the context of a MAP and injected with CFA [53]. These experiments show the extent to which T1 provides help to increase antibody production. Furthermore, these experiments show the feasibility of displaying the T1 and B epitopes in tandem on either the N- or C-terminus, or both. Polypeptide mixing experiments show whether T- and B-cell epitopes need to be linked (cis placement) on the same linear strand of the nanoparticle or if they can function as independent sequences (trans) in the same particle.

In addition to the potency assays described in Table 3 and 4, ELISpot Assays are performed to determine IFN-γ production in response to the T1 peptide stimulation of splenocytes in vitro. By using CD4+ or CD8+ antibodies, these cell types are eliminated in vitro to determine their role in IFN-γ production. ELISpot assays are compared with LSRII data acquired from the same cell harvest to establish a database of cell marker responses to P. berghei CSP epitope vaccines.

### Example 6

Inclusion of CD8+ T cell epitopes in SAPN vaccines. The P. berghei CSP CD8+ epitope SYPSAEKI (SEQ ID NO 14) has been shown to induce CTL in Balb/c mice [6, 54], and the involvement of CD8+ cells in immunity to malaria has been widely described in the irradiated sporozoite model of immunization [8, 55, 56]. Therefore, the CD8+ epitope is genetically fused to the core self-assembly polypeptide and using groups similar to those described above, it is determined whether this single epitope alone can induce protective immunity. If CD8+ T cell epitope peptides can also be presented by SAPN vaccines this shows their value for inclusion in the design for a P. falciparum CSP based vaccine and other future SAPN designs. Epitope density of these peptides does not influence the immune response, although the quantity may, because for these epitopes to be effective, the SAPN is taken up by DC or other antigen presenting cells (APC) and the epitopes processed and presented. The number and combination of CD4+ and CD8+ epitopes and B cell epitopes improve the potency of the vaccines, and the orientation of these epitopes affect their ability to be presented, and increase the memory cell production in the mice.

7.5 μg of non-T cell epitope containing particles confer complete protection and all the constructs give >50% protection. In this instance, the amount of SAPN used is reduced as the immunogen to 5.0, 2.5, 1 or 0.1 μg per dose. By these experiments it is possible to quantify the potency of each SAPN for the efficient presentation of B- and T cell epitopes. Because a reduced density of the B cell epitope increases potency, the optimum configuration of T and B cell peptides may not be determined from these experiments. It is possible to titrate the ratio of the different peptides within the best configuration, to determine the optimal ratio of T to B cell epitopes.

The T1 epitope is H-2^{d} restrictive so a potency assay is established for Balb/c mice. Because the protection level and antibody titers are similar for both mice strains, there are similar potency values. Groups (n=10) of Balb/c mice will be immunized with 7.5 μg (to be adjusted depending on potency studies) of each SAPN. Serum (for Ab titer determinations) are collected two weeks after each immunization and mice are challenged after the 3^{rd} immunization (see Tables 3 and 4).

### Example 7

Modify the prototype SAPN malaria vaccine that was protective in the mouse model to make it suitable for administration to humans. The core, self-assembly, coiled-coil domains of the prototype SAPN malaria vaccine are derived from the pentameric COMP sequence of rattus norvegicus and a trimeric coiled-coil that was designed de novo. The pentameric COMP sequence presents a risk for inducing autoimmunity because of amino acid identities between the human and rat COMP sequences. Additionally, the nanoparticle core is engineered to contain B cell and T cell epitopes that are specific to P. falciparum. The COMP pentamerization and the de novo-designed trimerization domain is replaced with analogous domains derived from the P. falciparum genome. The core nanoparticle constructed is tested for homogeneity, solubility and long-term storage, epitopes are engineered onto the amino terminus, it is re-tested for homogeneity, solubility and long term storage, and those that meet these criteria are tested for efficacy in protecting against live sporozoite challenge in the P. berghei model. The goal of this new SAPN design is to maximize both the malaria-specific immune response and biophysical properties to produce a homogenous and soluble formulation that is stable upon in long term storage.

SAPN rely on the natural “oligomerization domains” of the building block peptides for self-assembly (FIG. 1). Protein oligomerization domains are well-known in nature [57, 58]. Some of the best known examples are the GCN4 leucine zipper [59], fibritin [60], tetra-brachion [61] and Cartilage Oligomerization Matrix Protein (COMP) [46], representing dimeric, trimeric, tetrameric, and pentameric coiled-coils, respectively. Coiled-coils consist of two to five amphipathic α-helices that twist around one another to form a supercoil—much like strands of tread are used to make a strong rope. A search of the malaria specific genome database, PlasmoDB, reveals many oligomerization matrix proteins that occur naturally in Plasmodium and can be used as building blocks to provide a nanoparticle core comprised of pathogen-specific sequences.

Replacement of the pentamerization and trimerization sequences of the prototype core nanoparticle with oligomerization sequences from the P. falciparum genome. The PlasmoDB Data base (http://www.plasmodb.org/plasmo/) is searched for coiled-coil domains that can substitute for the domains of the SAPN based on the criteria that they should have hydrophobic residues at the first (a) and the fourth (d) position in the heptad repeats of a sequence of at least three heptads. The Plasmodium genome itself encodes for about 50 coiled-coil oligomerization domains. The preferred protein sequences that meet these criteria for trimeric coiled-coils are PF11_{—}0207, PF11_{—}0240 and PF14_{—}0535 (sequence numbers from PlasmoDB). Each of these is tested for its ability to substitute for the trimerization domain of the prototype core nanoparticle. Additionally, a trimeric coiled-coil consisting of fragments of these fused together is tested, the rationale being that this approach overcomes aggregation problems resulting from the repetitive sequences within the individual coiled-coil proteins.

The engineering of pentameric sequences from P. falciparum coiled-coil sequence is more difficult to achieve. Straight forward rules for predicting the pentameric oligomerization state are not available. Accordingly, none of the sequences in the PlasmoDB can be predicted to be a pentameric coiled-coil. However, as described by M. Lu and co-workers [62], i.e., replacement of all the a and d positions of the heptad repeat of a coiled-coil with tryptophan residues, can force it into a highly stable pentameric oligomerization state. For engineering a malaria Trp-zipper, a predicted coiled-coil sequences was chosen from PlasmoDB that is similar to the sequence used by Liu et al [62] in Table 6:

TABLE 6
PFI1180w
NNFDNYNNNCDNYYNNFDNYNNNFDNYNNNFDNYNNNFDNYNNN
a d a d a d a d a d a d heptad position
(SEQ ID NO 15)
PFF0535c
HNHYDNRYNHHDNRYNHHDNRYNHHDNRYNHHDNRYNHHDNRYNNK
(SEQ ID NO 16)

These highly repetitive sequences contain largely aromatic residues at coiled-coil core positions (indicated above the sequences), either phenylalanine and tyrosine (PFI1180w) or histidine and tyrosine (PFF0535c). These bulky aromatic residues are replaced by the other (more) bulky aromatic residue tryptophan, and hence malaria Trp-zippers is engineered from very homologous coiled-coil sequences that will presumably form pentameric coiled-coils. The engineering of Trp-residues into the sequence may destroy possible T cell epitopes. However, B cell epitopes are most likely be retained, since the surface of the new coiled-coil looks similar to the surface of the original coiled-coil. Consequently it has been shown, that neutralizing antibodies for the trimeric coiled-coil of SARS can be obtained from a dimeric coiled-coil antigen [63].

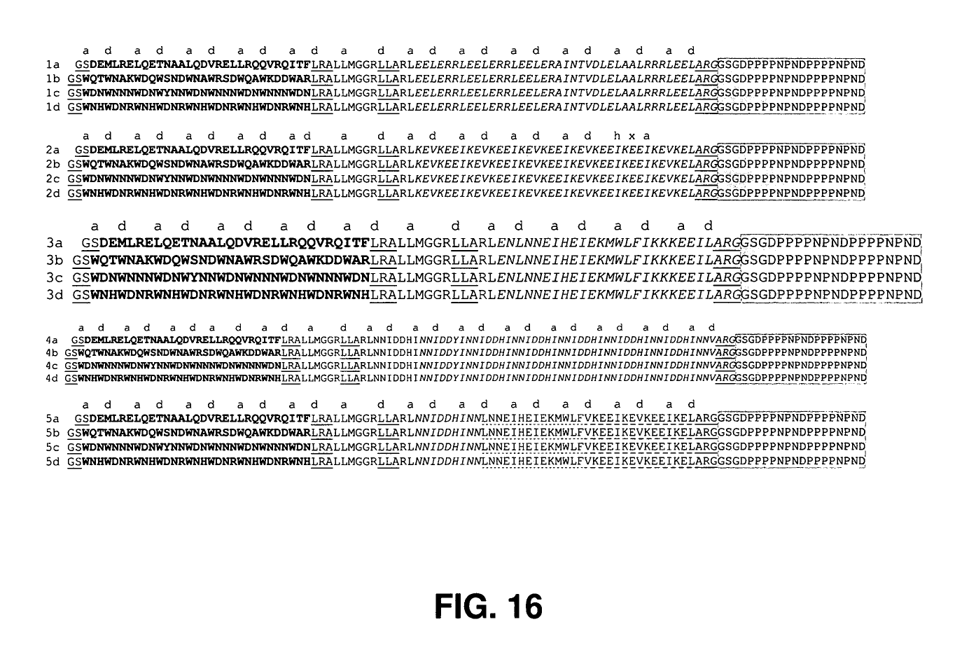

Each of the new trimeric coiled-coils are tested with each of four different pentamerization domains: 1) the original pentamerization sequence, 2) the published tryptophan zipper [62], and the P. falciparum coiled-coil sequences, 3) PFI1180w and 4) PFF0535c. These pairings of pentamerization with trimerization domains sum to a total of 20 permutations (see sequences in FIG. 16). These 20 sequences are constructed, expressed, purified, assembled and tested for homogeneity, solubility and stability in solution. The proper peptide sequence is verified by MALDI-TOF, and those sequences that form nanoparticles of the expected size and shape, as judged by EM micrographs, and remain soluble for at least one month at a minimal protein concentration of 0.1 mg/ml, as judged by DLS, are tested for immunogenicity and efficacy in the P. berghei mouse malaria model. The lead core self-assembly domain is modified with the P. falciparum CSP repeat and tested for ability to stimulate an antibody response to it, with and without T cell epitopes (known and predicted) from the P. falciparum genome.

Effect of coiled-coil sequences containing CD4+ and CD8+ epitopes predicted from the P. falciparum genome on protective efficacy in HLA transgenic mice. Potent and promiscuous T-cell epitopes are predicted within all of the other predicted malaria coiled-coil sequences and use these for the core design of the SAPNs as outlined above for the sequences PF11_{—}0207, PF11_{—}0240 and PF14_{—}0535. If they do not form trimeric coiled-coils then they are modified to do so by putting leucine residues in both the a and d positions of the heptad repeat. This has been shown to induce trimerization of coiled-coil sequences [42, 43, 64-68]. As for the pentamer design, however, this approach can only retain the B cell—but not the T cell epitopes. A pentamerization/trimerization combination derived from malaria, and/or designed, is found as the basis for core self-assembly for producing SAPN vaccines that are homogenous, soluble and stable. This is expected because only the heptad repeats of the pentameric and trimeric coiled-coils that do not interact are exchanged (bold in FIG. 16); the linker residues (plain font in FIG. 16) that are involved in interactions between pentamer and trimer, are not changed. The trimer was successfully replaced with a different sequence using the same approach (see FIG. 4). In the fully assembled SAPN, the trimer (italicized segment in FIG. 16) does not interact with any other part of the SAPN except with the other chains of the trimer itself. Hence, if the new italicized sequence forms a trimeric coiled-coil it will most likely be able to replace the old trimeric coiled-coil without abolishing SAPN formation. The same holds true for the bold pentameric coiled-coil. Therefore, the novel Trp-zipper motif enables SAPN designs having one pentameric coiled-coil replaced by another (see the similarity of two pentameric domains in FIG. 15).

Individual oligomerization domains of the SAPN are be exchanged and also coiled-coil oligomerization states can rather accurately be predicted. Still, proper SAPN folding cannot be 100% guaranteed. If the SAPN with the new oligomerization domains do not fold as expected, the oligomerization domains are separately investigated to verify the oligomerization state. Then, in a cyclic procedure using computer modeling and verification by AUC and X-ray structure analysis, the coiled-coil sequences are optimized until the desired oligomerization state has been achieved.

Core design. The sequences show in FIG. 16 show the pentamer (bold), the linker region (regular font), the trimer (italicized), the epitope (highlighted) and the restriction sites (underscored). The heptad repeat pattern for the pentamer and the trimer is indicated above the sequences as a and d positions. The chimeric protein shown in FIG. 16 as 5a-c, comprises of fragments of PF11_{—}0207 (italicized), PF11_{—}0240 (dotted-underlined) and PF14_{—}0535 (dashed-underlined). The code represents the combination of pentamers and trimers according to the following rules:

Trimers: 1x, original sequence (without cysteins and lysines); 2x, PF11_{—}0207; 3x, PF11_{—}0240; 4x, PF14_{—}0535; 5x, chimeric of PF11_{—}0207, PF11_{—}0240 and PF14_{—}0535.

Pentamers: xa, original sequence (without cysteins and lysines); xb, Trp-zipper; xc, W-substituted PFI1180w; xd, W-substituted PFF0535c.

Restriction sites: GS (Barn HI); LRA (BssHII); LLA (NheI); RRL (AatII); LE (XhoI); VD (Sal I); ARG (XmaI); at start, not shown (NcoI); after stop codons, not shown (Eco RI).
To replace the pentamer, use BamHI and BssHII;
To replace the linker region use BssHII and AatII;
To replace the trimer use NheI and XmaI;
To extend the trimer use Xho I and Sal I;
To replace the epitope use XmaI and EcoRI;
To insert T-cell epitope at N-terminus use NcoI and BamHI.

### Example 8

Design, produce and test a P. falciparum SAPN vaccine based on the known immunodominant CSP B-cell epitope sequence (NANP)₃ (SEQ ID NO 93), the universal CSP Th epitope, T*(EYLNKIQNSLSTEWSPCSVT) (SEQ ID NO 8), the CSP CD4+ T cell epitope, T1 ([DPNANPNV]₂) (SEQ ID NO 9), and promiscuous T cell epitopes predicted from proteins expressed at high levels in the P. falciparum sporozoite. The only effective vaccine (RTS,S) against malaria today is based on the PfCSP. This immunogen consists of 16 repeats and the C-terminal end of the PfCSP, and both T and B cell epitopes have been identified in the sequence. It is delivered as a particulate antigen with an adjuvant (AS02A). The immunodominant B cell epitope of PfCSP is contained within (NANP)₃ [70]. Several T cell epitopes have been identified: T*, a “universal” T helper epitope, is located in a conserved sequence in the C-terminal portion of PfCSP (and RTS,S). It has been shown to be recognized by several stains of mice [24], non-human primates [71] and humans with diverse class II haplotypes [72]. Another CD4+ T-cell epitope, (DPNANPNV)₂ (SEQ ID NO 9), is located at the beginning of the central repeat region.

Mice are not susceptible to P. falciparum parasites; therefore, challenge experiments with P. falciparum are not feasible. As an alternative, human malaria vaccine, HLA transgenic mice (DRB1*0301 or DRB1*0401) are used as a model for the human response to P. falciparum T cell epitopes following vaccination with a SAPN vaccine that additionally displays the P. berghei B cell epitope. The effectiveness of the P. falciparum T cell epitopes to help produce antibodies to the P. berghei B cell epitope is determined. Both titer and affinity of the antibodies are measured. Additionally, cellular immune responses (IL-2, IL-4, IL-5, INF-gamma, TNFalpha) are measured in SAPN-vaccinated mice that are bitten by P. falciparum-infected mosquitoes; even though the mice will not become infected, they should generate a cellular immune response. Another group of mice are challenged with P. berghei to quantify the help provided by the T cell epitopes on the SAPN for potentiating the protective antibody response to the P. berghei B cell epitopes.

HLA transgenic mice are a better model for human T cell response than outbred murine models because murine MHC are not directly equivalent to HLA with regard to epitope restrictions. Also, a direct correlation has been found between T cell responses in infected individuals and T cell responses induced in immunized HLA transgenic mice. Furthermore, following influenza infection, the pattern of epitope recognition in HLA transgenic mice has strong similarity to that in humans [73], indicating that events of antigen processing, presentation and recognition are well-conserved between species.

Effectiveness of P. falciparum T cell epitope peptides to supply help for generating an immune response to P. berghei B cell epitopes in transgenic HLA mice. The optimal configuration of the T1 and T* on SAPN that display the P. berghei B-cell epitope (DPPPNPND)₂D (SEQ ID NO 17) are determined. Optimization of core size and ratio of B cell to T cell epitope peptides are used to choose the best extender length and density of peptides. Groups of C57BL/6 (n=20), transgenic DRB1*0301 and DRB1*0401 mice are immunized with 10 μg of each SAPN at day 0, 14, 28. Serum for Ab titer determinations are collected. Two weeks after the third immunization, 10 mice in each group are sacrificed and lymph nodes and spleens removed to evaluate phenotypic marker expression (IL-2, IL-4, IL5, INFg, TNF-α) in cells (ELISpot) and in serum (Luminex 200 xMAP). The remaining 10 mice are challenged with P. berghei sporozoites 2 weeks after the third dose to determine the potency of the SAPN for inducing a protective immune response.

SAPN with P. falciparum CSP T and B cell epitopes. The B cell epitope from P. berghei are replaced with the B cell epitope from P. falciparum (NANP)₃, (SEQ ID NO. 93) and the antibody response to the P. falciparum B cell epitope are determined in the same groups of C57BL/6, transgenic DRB1*0301 and DRB1*0401 mice. Antibody and cellular cytokines are evaluated; however, because the mice cannot be infected with P. falciparum parasites, there is no infection. However, 5 mice in each group are challenged by the bites of 20 P. falciparum infected mosquitoes at 2 weeks, and then another 5 mice, 4 weeks after the third dose of vaccine. CSP specific immune markers increase as a result of vaccination and parasite challenge.

Immunological Analysis: Serum (100 μl) is sampled from all mice before immunization (pre-bleed) and 2 wks after each immunization or before each parasite challenge. Ab titer is determined by ELISA and Ig isotype determined using Luminex 200 xMAP Technology. Two wks post 3^{rd} immunization, 10 mice in each group are sacrificed and lymph nodes and spleens are removed cells evaluated for phenotypic marker expression (IL-2, IL5, INFg, TNF-α). This is repeated on the remaining two sets of 5 mice in each group following sporozoite challenge.

SAPN construct with T cell epitopes predicted from highly expressed sequences in P. falciparum sporozoite stage. To broaden the immune response, algorithms are used to predict T cell epitopes present in P. falciparum sequences that are highly expressed in the sporozoite. These T cell epitopes are tested in a variety of configurations, which include in combination with the known T cell epitopes, and in multiple epitopes displayed in tandem on the N- or C-terminus, or both. If the helper effect of these epitopes become saturated in the model, the number of self-assembly polypeptides per SAPN that contain the T cell epitopes are reduced to titrate down their effect.

Other embodiments and uses of the invention will be apparent to those skilled in the art from consideration of the specification and practice of the invention disclosed herein. All references cited herein, including all publications, U.S. and foreign patents and patent applications (including International Application No. PCT/IB04/00423 by Peter Burkhard filed Feb. 16, 2004 are specifically and entirely incorporated by reference. It is intended that the specification and examples be considered exemplary only with the true scope and spirit of the invention indicated by the following claims. Furthermore, the term “comprising of” includes the terms “consisting of” and “consisting essentially of.”

### REFERENCES CITED

All references are incorporated herein in their entirety.

1. Alonso, P. L., et al., Duration of protection with RTS,S/AS02A malaria vaccine in prevention of Plasmodium falciparum disease in Mozambican children: single-blind extended follow-up of a randomized controlled trial. Lancet, 2005. 366(9502): p. 2012-8.

2. Alonso, P. L., et al., Efficacy of the RTS,S/AS02A vaccine against Plasmodium falciparum infection and disease in young African children: randomized controlled trial. Lancet, 2004. 364(9443): p. 1411-20.

3. Ballou, W. R., Malaria vaccines in development. Expert Opin Emerg Drugs, 2005. 10(3): p. 489-503.

4. Eichinger, D. J., et al., Circumsporozoite protein of Plasmodium berghei: gene cloning and identification of the immunodominant epitopes. Mol Cell Biol, 1986. 6(11): p. 3965-72.

5. Tam, J. P., et al., Incorporation of T and B epitopes of the circumsporozoite protein in a chemically defined synthetic vaccine against malaria. J Exp Med, 1990. 171(1): p. 299-306.

6. Romero, P., et al., Cloned cytotoxic T cells recognize an epitope in the circumsporozoite protein and protect against malaria. Nature, 1989. 341(6240): p. 323-6.

7. Tam, J. P., Synthetic peptide vaccine design: synthesis and properties of a high-density multiple antigenic peptide system. Proc Natl Acad Sci USA, 1988. 85(15): p. 5409-13.

8. Zavala, F., et al., Synthetic peptide vaccine confers protection against murine malaria. J Exp Med, 1987. 166(5): p. 1591-6.

9. Chai, S. K., et al., Immunogenic properties of multiple antigen peptide systems containing defined T and B epitopes. J Immunol, 1992. 149(7): p. 2385-90.

10. Gluck, R. and I. C. Metcalfe, New technology platforms in the development of vaccines for the future. Vaccine, 2002. 20 Suppl 5: p. B10-6.

11. Zurbriggen, R., Immunostimulating reconstituted influenza virosomes. Vaccine, 2003. 21(9-10): p. 921-4.

12. Moser, C., I. C. Metcalfe, and J. F. Viret, Virosomal adjuvanted antigen delivery systems. Expert Rev Vaccines, 2003. 2(2): p. 189-96.

13. Takahashi, H., et al., Induction of CD8+ cytotoxic T cells by immunization with purified HIV-1 envelope protein in ISCOMs. Nature, 1990. 344(6269): p. 873-5.

14. Sjolander, A., J. C. Cox, and I. G. Barr, ISCOMs: an adjuvant with multiple functions. J Leukoc Biol, 1998. 64(6): p. 713-23.

15. Vajdy, M. and D. T. O'Hagan, Microparticles for intranasal immunization. Adv Drug Deliv Rev, 2001. 51(1-3): p. 127-41.

16. Schirmbeck, R., et al., Selective stimulation of murine cytotoxic T cell and antibody responses by particulate or monomeric hepatitis B virus surface (S) antigen. Eur J Immunol, 1994. 24(5): p. 1088-96.

17. Wagner, R., et al., Construction, expression, and immunogenicity of chimeric HIV-1 virus-like particles. Virology, 1996. 220(1): p. 128-40.

18. Roth, J. F., The yeast Ty virus-like particles. Yeast, 2000. 16(9): p. 785-95.

19. Schiller, J. T. and D. R. Lowy, Papillomavirus-like particle vaccines. J Natl Cancer Inst Monogr, 2001(28): p. 50-4.

20. Schodel, F., et al., Hybrid hepatitis B virus core antigen as a vaccine carrier moiety: I. presentation of foreign epitopes. J Biotechnol, 1996. 44(1-3): p. 91-6.

21. Schodel, F., et al., Immunity to malaria elicited by hybrid hepatitis B virus core particles carrying circumsporozoite protein epitopes. J Exp Med, 1994. 180(3): p. 1037-46.

22. Oliveira-Ferreira, J., et al., Immunogenicity of Ty-VLP bearing a CD8(+) T cell epitope of the CS protein of P. yoelii: enhanced memory response by boosting with recombinant vaccinia virus. Vaccine, 2000. 18(17): p. 1863-9.

23. Plebanski, M., et al., Protection from Plasmodium berghei infection by priming and boosting T cells to a single class I-restricted epitope with recombinant carriers suitable for human use. Eur J Immunol, 1998. 28(12): p. 4345-55.

24. Birkett, A., et al., A modified hepatitis B virus core particle containing multiple epitopes of the Plasmodium falciparum circumsporozoite protein provides a highly immunogenic malaria vaccine in preclinical analyses in rodent and primate hosts. Infect Immun, 2002. 70(12): p. 6860-70.

25. Oliveira, G. A., et al., Safety and enhanced immunogenicity of a hepatitis B core particle Plasmodium falciparum malaria vaccine formulated in adjuvant Montanide ISA 720 in a phase I trial. Infect Immun, 2005. 73(6): p. 3587-97.

26. Walther, M., et al., Safety, immunogenicity and efficacy of a pre-erythrocytic malaria candidate vaccine, ICC-1132 formulated in Seppic ISA 720. Vaccine, 2005. 23(7): p. 857-64.

27. Wilson, N. S., D. El-Sukkari, and J. A. Villadangos, Dendritic cells constitutively present self antigens in their immature state in vivo and regulate antigen presentation by controlling the rates of MHC class II synthesis and endocytosis. Blood, 2004. 103(6): p. 2187-95.

28. Wilson, N. S. and J. A. Villadangos, Lymphoid organ dendritic cells: beyond the Langerhans cells paradigm. Immunol Cell Biol, 2004. 82(1): p. 91-8.

29. Wilson, N. S., et al., Most lymphoid organ dendritic cell types are phenotypically and functionally immature. Blood, 2003. 102(6): p. 2187-94.

30. Reddy, S. T., M. A. Swartz, and J. A. Hubbell, Targeting dendritic cells with biomaterials: developing the next generation of vaccines. Trends Immunol, 2006. 27(12): p. 573-9.

31. Reddy, S. T., et al., In vivo targeting of dendritic cells in lymph nodes with poly(propylene sulfide) nanoparticles. J Control Release, 2006. 112(1): p. 26-34.

32. Noad, R. and P. Roy, Virus-like particles as immunogens. Trends Microbiol, 2003. 11(9): p. 438-44.

33. Bachmann, M. F., et al., The influence of antigen organization on B cell responsiveness. Science, 1993. 262(5138): p. 1448-51.

34. Bachmann, M. F. and R. M. Zinkernagel, The influence of virus structure on antibody responses and virus serotype formation. Immunol Today, 1996. 17(12): p. 553-8.

35. Dintzis, H. M., R. Z. Dintzis, and B. Vogelstein, Molecular determinants of immunogenicity: the immunon model of immune response. Proc Natl Acad Sci USA, 1976. 73(10): p. 3671-5.

36. Dintzis, R. Z., B. Vogelstein, and H. M. Dintzis, Specific cellular stimulation in the primary immune response: experimental test of a quantized model. Proc Natl Acad Sci USA, 1982. 79(3): p. 884-8.

37. Vogelstein, B., R. Z. Dintzis, and H. M. Dintzis, Specific cellular stimulation in the primary immune response: a quantized model. Proc Natl Acad Sci USA, 1982. 79(2): p. 395-9.

38. Baschong, W., et al., Repetitive versus monomeric antigen presentation: direct visualization of antibody affinity and specificity. J Struct Biol, 2003. 143(3): p. 258-62.

39. Liu, W. and Y. H. Chen, High epitope density in a single protein molecule significantly enhances antigenicity as well as immunogenicity: a novel strategy for modern vaccine development and a preliminary investigation about B cell discrimination of monomeric proteins. Eur J Immunol, 2005. 35(2): p. 505-14.

40. Raman, S., et al., Structure-based design of peptides that self-assemble into regular polyhedral nanoparticles. Nanomedicine, 2006. 2(2): p. 95-102.

41. Johnson, J. E. and V. S. Reddy, Biggest virus molecular structure yet! Nat Struct Biol, 1998. 5(10): p. 849-54.

42. Burkhard, P., S. Ivaninskii, and A. Lustig, Improving coiled-coil stability by optimizing ionic interactions. J Mol Biol, 2002. 318(3): p. 901-10.

43. Burkhard, P., M. Meier, and A. Lustig, Design of a minimal protein oligomerization domain by a structural approach. Protein Sci, 2000. 9(12): p. 2294-301.

44. Burkhard, P., Peptidic Nanoparticles as Drug Delivery and Antigen Display Systems. PCT patent application, 2004: p. WO 2004/071493.

45. Sanner, M. F., et al., Visualizing Nature at Work from the Nano to the Macro Scale. Nanobiotechnology, 2005. 1(1): p. 7-22.

46. Malashkevich, V. N., et al., The crystal structure of a five-stranded coiled coil in COMP: a prototype ion channel? Science, 1996. 274(5288): p. 761-5.

47. Raman, S. S., et al., Role of aspartic acid in collagen structure and stability: A molecular dynamics investigation. J Phys Chem B Condens Matter Mater Surf Interfaces Biophys, 2006. 110(41): p. 20678-85.

48. Casey, J. L., et al., Antibodies to malaria peptide mimics inhibit Plasmodium falciparum invasion of erythrocytes. Infect Immun, 2004. 72(2): p. 1126-34.

49. Feldmann, M., J. G. Howard, and C. Desaymard, Role of antigen structure in the discrimination between tolerance and immunity by b cells. Transplant Rev, 1975. 23: p. 78-97.

50. Mond, J. J., et al., Analysis of B cell activation requirements with TNP-conjugated polyacrylamide beads. J Immunol, 1979. 123(1): p. 239-45.

51. Jegerlehner, A., et al., A molecular assembly system that renders antigens of choice highly repetitive for induction of protective B cell responses. Vaccine, 2002. 20(25-26): p. 3104-12.

52. Liu, W., et al., High epitope density in a single recombinant protein molecule of the extracellular domain of influenza A virus M2 protein significantly enhances protective immunity. Vaccine, 2004. 23(3): p. 366-71.

53. Migliorini, P., B. Betschart, and G. Corradin, Malaria vaccine: immunization of mice with a synthetic T cell helper epitope alone leads to protective immunity. Eur J Immunol, 1993. 23(2): p. 582-5.

54. Allsopp, C. E., et al., Comparison of numerous delivery systems for the induction of cytotoxic T lymphocytes by immunization. Eur J Immunol, 1996. 26(8): p. 1951-9.

55. Tsuji, M. and F. Zavala, Peptide-based subunit vaccines against pre-erythrocytic stages of malaria parasites. Mol Immunol, 2001. 38(6): p. 433-42.

56. Sun, P., et al., Protective immunity induced with malaria vaccine, RTS,S, is linked to Plasmodium falciparum circumsporozoite protein-specific CD4+ and CD8+ T cells producing IFN-gamma. J Immunol, 2003. 171(12): p. 6961-7.

57. Burkhard, P., S. V. Strelkov, and J. Stetefeld, Coiled coils: a highly versatile protein folding motif. Trends Cell Biol, 2001. 11(2): p. 82-8.

58. Lupas, A., Prediction and analysis of coiled-coil structures. Methods Enzymol, 1996. 266: p. 513-25.

59. O'Shea, E. K., et al., X-ray structure of the GCN4 leucine zipper, a two-stranded, parallel coiled coil. Science, 1991. 254(5031): p. 539-44.

60. Tao, Y., et al., Structure of bacteriophage T4 fibritin: a segmented coiled coil and the role of the C-terminal domain. Structure, 1997. 5(6): p. 789-98.

61. Stetefeld, J., et al., Crystal structure of a naturally occurring parallel right-handed coiled coil tetramer. Nat Struct Biol, 2000. 7(9): p. 772-6.

62. Liu, J., et al., Atomic structure of a tryptophan-zipper pentamer. Proc Natl Acad Sci USA, 2004. 101(46): p. 16156-61.

63. Tripet, B., et al., Template-based coiled-coil antigens elicit neutralizing antibodies to the SARS-coronavirus. J Struct Biol, 2006. 155(2): p. 176-94.

64. Tripet, B., et al., Effects of side-chain characteristics on stability and oligomerization state of a de novo-designed model coiled-coil: 20 amino acid substitutions in position “d”. J Mol Biol, 2000. 300(2): p. 377-402.

65. Wagschal, K., et al., The role of position a in determining the stability and oligomerization state of alpha-helical coiled coils: 20 amino acid stability coefficients in the hydrophobic core of proteins. Protein Sci, 1999. 8(11): p. 2312-29.

66. Woolfson, D. N. and T. Alber, Predicting oligomerization states of coiled coils. Protein Sci, 1995. 4(8): p. 1596-607.

67. Wolf, E., P. S. Kim, and B. Berger, MultiCoil: a program for predicting two- and three-stranded coiled coils. Protein Sci, 1997. 6(6): p. 1179-89.

68. Harbury, P. B., et al., High-resolution protein design with backbone freedom. Science, 1998. 282(5393): p. 1462-7.

69. Burkhard, P., et al., The coiled-coil trigger site of the rod domain of cortexillin I unveils a distinct network of interhelical and intrahelical salt bridges. Structure, 2000. 8(3): p. 223-30.

70. Chappel, J. A., et al., Molecular dissection of the human antibody response to the structural repeat epitope of Plasmodium falciparum sporozoite from a protected donor. Malar J, 2004. 3: p. 28.

71. Langermans, J. A., et al., Effect of adjuvant on reactogenicity and long-term immunogenicity of the malaria Vaccine ICC-1132 in macaques. Vaccine, 2005. 23(41): p. 4935-43.

72. Calvo-Calle, J. M., et al., A linear peptide containing minimal T- and B-cell epitopes of Plasmodium falciparum circumsporozoite protein elicits protection against transgenic sporozoite challenge. Infect Immun, 2006. 74(12): p. 6929-39.

73. Hu, N., et al., Highly conserved pattern of recognition of influenza A wild-type and variant CD8+ CTL epitopes in HLA-A2+ humans and transgenic HLA-A2+/H2 class I-deficient mice. Vaccine, 2005. 23(45): p. 5231-44.

1-14. (canceled)

15. A vaccine for the prevention or treatment of malaria, wherein said vaccine comprises:
a self-assembling polypeptide comprising:
a pentameric domain;
a trimeric domain; and
a linker that joins the pentameric domain and the trimeric domain; and
an epitope of an antigen capable of inducing a protective immune response in a mammal susceptible to infection by a malaria parasite.

16. The vaccine of claim 15, wherein the self-assembling polypeptide is a continuous chain comprising peptide oligomerizations of the pentameric domain and the trimeric domain.

17. The vaccine of claim 15, wherein the epitope is selected from one or more of the antigens and proteins set forth in Table 2.

18. The vaccine of claim 15, wherein the sequence is selected from one or more of the sequences set forth in Table 3.

19. The vaccine of claim 15, further comprising a pharmaceutically acceptable carrier.

20. The vaccine of claim 15, wherein the antigen is a circumsporozoite protein of P. falciparum.

21. A method for vaccinating against infection from a malaria parasite comprising:
administering a functionalized self-assembling polypeptide nanoparticle comprising:
a self-assembling core; and
an epitope fused to the self-assembling core, wherein the self-assembling core comprises:
a pentameric coiled-coil domain;
a trimeric coiled-coil domain; and
a linker joining the pentameric coiled-coil domain and the trimeric coiled-coil domain wherein the epitope generates an immunologically protective reaction against infection by a malaria parasite when administered to a mammal.

22. The method of claim 21, wherein the nanoparticle is administered without an adjuvant.

23. The method of claim 21, wherein the epitope is PfCSP B-cell epitope sequence, (NANP)₃(SEQ ID NO. 93).

24. The method of claim 21, wherein the epitope is PfCSP B-cell epitope sequence, (NANP)₄ (SEQ ID NO. 94).

25. The method of claim 21, wherein the epitope is a universal epitope comprising the sequence of SEQ ID NO. 8.

26. The method of claim 21, wherein the epitope comprises the sequence of SEQ ID NO. 9.

27. (canceled)

28. The method of claim 21, wherein said nanoparticle has a diameter of about 20 nm.

29. The method of claim 21, wherein the epitope comprises an antigen of a malaria parasite.

30. The method of claim 29, wherein the antigen is derived from a protein of P. falciparum.

31. The method of claim 29, wherein the antigen is circumsporozoite protein.

32. The method of claim 29, wherein the antigen is derived from the circumsporozoite protein of P. vivax.

33-50. (canceled)

51. A method for vaccinating against infection from a malaria parasite comprising:
administering a functionalized self-assembling polypeptide nanoparticle comprising: a self-assembling core; and
PanDR binding peptide HTL epitope fused to the self-assembling core, wherein the self-assembling core comprises:
a pentameric coiled-coil domain;
a trimeric coiled-coil domain; and
a linker joining the pentameric coiled-coil domain and the trimeric coiled-coil domain wherein the epitope generates an immunologically protective reaction against infection by a malaria parasite when administered to a mammal.

52. The method of claim 51, wherein the nanoparticle is administered without an adjuvant.

53. The method of claim 51, wherein the PanDR binding peptide HTL epitope is selected from the group of sequences consisting of:

| | |
|---|---|
| AKFVAAWTLKAAA; | (SEQ ID NO 141) |
| AKFVAANTLKAAA; | (SEQ ID NO 142) |
| AKFVAAYTLKAAA; | (SEQ ID NO 143) |
| AKFVAAKTLKAAA; | (SEQ ID NO 144) |
| AKFVAAHTLKAAA; | (SEQ ID NO 145) |
| and, | |
| AKFVAAATLKAAA. | (SEQ ID NO 146) |
| (Canceled) | |

54.
